(19) United States
(12) Patent Application Publication
(10) Pub. No.: US 2024/0175054 A1
(43) Pub. Date: May 30, 2024

(54) COMPOSITION AND METHODS FOR THE TREATMENT OF FABRY DISEASE

(71) Applicant: TAKEDA PHARMACEUTICAL COMPANY LIMITED, Osaka (JP)

(72) Inventors: Rizwana ISLAM, Winchester, MA (US); Mugdha Ravindra DESHPANDE, Arlington, MA (US); Madhusudan NATARAJAN, Waban, MA (US); Yung Hee PARK, Arlington, MA (US); Vivan CHOI, Cambridge, MA (US)

(21) Appl. No.: 18/547,809

(22) PCT Filed: Feb. 25, 2022
(86) PCT No.: PCT/US2022/017998

§ 371 Date: Aug. 24, 2023

Related U.S. Application Data
(60) Provisional application No. 63/154,485, filed on Feb. 26, 2021.

(51) Int. Cl. C12N 15/86; A61P 43/00; C12N 9/40

(52) U.S. Cl. CPC C12N 15/86; A61P 43/00; C12N 9/2465; C12Y 302/01022; C12N 2750/14122; C12N 2750/14143; C12N 2830/50

(57) ABSTRACT

The present disclosure provides, among other things, a method of treating Fabry disease in a subject, the method comprising administering to a subject in need thereof a recombinant adeno-associated viral vector (rAAV) packaged in AAV capsid having broad tissue tropism, the vector comprising: (a) a 5′ inverted terminal repeat; (b) a ubiquitous promoter; (c) a nucleotide sequence encoding wild-type α-GAL enzyme or a variant thereof; (d) optionally a woodchuck hepatitis virus posttranscriptional regulatory element (WPRE); (e) a poly A; and (d) a 3′ITR.

### CROSS-REFERENCE TO RELATED APPLICATIONS

This application claims priority to, and the benefit of, U.S. provisional application No. 63/154,485, filed on Feb. 26, 2021, the contents of which is hereby incorporated by reference in its entirety.

### INCORPORATION-BY-REFERENCE OF SEQUENCE LISTING

The present specification makes reference to a Sequence Listing (submitted electronically as a .txt file named MIL-014WO_ST25 on Feb. 25, 2022). The .txt file was generated on Feb. 24, 2022 and is 177 KB in size. The entire contents of the sequence listing are herein incorporated by reference.

### BACKGROUND

Fabry disease is rare a progressive congenital metabolic disease caused by a deficiency in the lysosomal enzyme α-galactosidase A (α-GAL) as a result of a mutation in the GLA gene. If left untreated, Fabry patients have a reduced life expectancy, often dying around the age of forty or fifty due to vascular disease affecting the kidneys, heart and/or central nervous system.

Lack of α-GAL enzyme activity results in the progressive, systematic accumulation of its primary substrate, globotriaosylceramide (GB3) and its deacetylated soluble form, globotriaosylsphingosine (lysoGb3), resulting in a myriad of health issues including one or more of renal disease, cardiac disease, and/or cerebrovascular disease, with reduced life expectancy. Depending on the mutation and residual α-GAL enzyme level, the disease presents as a classical early-onset Fabry disease in childhood/adolescence or as an attenuated (adult) form later in life. Classical Fabry disease occurs when residual enzyme activity is <5% (Arends M, et al. (2017) PLoS ONE 12(8): e0182379.) and typically occurs in males.

Fabry disease is also associated with the development of pain. Pain is possibly caused by the deposition of lipids in the dorsal root ganglia and sympathetic ganglia, or by small fiber neuropathy. Generally, the pain is either chronic or episodic. Episodic pain in Fabry disease, termed “Fabry crises,” typically begins in the extremities and radiates proximally, and may be triggered by exercise, illness, temperature changes, or other physical and emotional stresses. This neuropathic pain is also associated with a lack of temperature perception.

The specific treatment of Fabry disease that is currently approved is enzyme replacement therapy (“ERT”) which involves treatment of patients with either of two versions of recombinant human α-GAL, agalsidase alfa, which is produced by cultured human cell lines; and agalsidase beta, which is produced by Chinese hamster ovary cells transduced with GLA gene. While ERT is effective in many cases, this treatment requires life-long intravenous administration of α-GAL every two weeks. ERT resolves symptoms associated with Fabry disease but is not curative and does not stop disease progression. For example, the two α-GAL products discussed above have not been shown to substantially reduce the risk of stroke, the myocardium responds slowly to treatment, and the elimination of lipid deposits from some cell types in the kidney is limited. The insufficient pharmacologic response is largely due to the short circulatory half-life of the enzyme and suboptimal cellular delivery. Thus, there remains a need for therapies for treating Fabry disease that can stop disease progression and potentially be curative.

### SUMMARY OF THE INVENTION

The present application discloses methods and compositions for the treatment and/or prevention of Fabry disease. The present disclosure provides, in part, a gene therapy approach using recombinant adeno associated viral vectors (rAAV) to mediate transfer and expression of the GLA gene. This application is based on the discovery that gene delivery vehicles such as a rAAV vector that has broad tissue tropism and utilizes a ubiquitous promoter to drive widespread gene expression results in sustained high levels of protein expression and robust protein exposure to a wide range of tissues and/or decrease in GB3 or lysoGb3 levels. This application is also based on the discovery that codon optimized or engineered variants of GLA delivered using a rAAV vector with broad or tissue specific tropism and utilizing a ubiquitous or tissue specific promoter result in an increase in α-GAL activity in vivo, and/or a decrease in lysoGb3 or GB3 levels in vivo. Additionally, this gene delivery approach to drive expression of GLA variants that encode α-GAL protein with increased half-life and improved cellular uptake provides further increases in α-GAL exposure in key target tissues. Collectively, these discoveries allow for the delivery vehicles described herein to achieve broad tissue distribution of administered transgenes, and also allows better treatment outcomes. The delivery vehicles comprising the GLA sequences described herein are particularly useful for the treatment of Fabry disease.

Described herein are methods and compositions for effective delivery of α-Galactosidase A (GLA) gene into cells of a subject in need thereof. The delivered GLA transgene results in expression of α-GAL protein. The present disclosure is based, in part, on the development of a recombinant adeno associated viral (rAAV) vector that comprises α-Galactosidase A (GLA) gene, among other things, and which demonstrates robust α-GAL protein expression once present in a cell. The present disclosure is based, at least in part, on the surprising discovery that a construct comprising a GLA gene under a ubiquitous promoter and packaged in a rAAV capsid with broad tissue tropism results in persistent α-GAL protein expression and robust tissue biodistribution in the kidney, heart, gastrointestinal tract, brain, and peripheral neurons even at a low dose. The broad distribution obtained using this vector allows for efficient delivery of α-GAL to tissues that are affected by Fabry disease, and consequently allows for a robust therapeutic result.

The rAAV vectors described herein can be used with either a GLA gene having a wild type sequence (SEQ ID NO: 3) or a GLA gene having a modified sequence described herein. Such modified GLA sequences include, for example, codon optimized GLA and/or engineered variants of GLA. The rAAV vectors described herein allows for substrate clearance of globotriaosylsphingosine (lysoGb3) and/or globotriaosylceramide (GB3) in various tissues.

As described in more detail below, the gene therapy system described herein results in overall improvement in health as evidenced by gain in body mass, improved kidney function, and neurological symptoms in Fabry disease mouse models and is further expected to elicit the same in humans. The methods and compositions provided herein can be used to achieve sustained expression of GLA in a wide variety of tissues that are affected in Fabry disease. Thus, the present application provides composition and methods that are highly effective in the treatment of Fabry disease and alleviation of associated symptoms.

In some aspects, a recombinant adeno-associated virus (rAAV) vector with broad tissue tropism is provided, said vector comprising: (a) a 5′ inverted terminal repeat (ITR); (b) a ubiquitous promoter; (c) a nucleotide sequence encoding α-GAL enzyme; (d) a poly A; and (e) a 3′ ITR.

In some aspects, a recombinant adeno-associated virus (rAAV) vector with broad tissue tropism is provided, said vector comprising: (a) a 5′ inverted terminal repeat (ITR); (b) a ubiquitous promoter; (c) a nucleotide sequence encoding α-GAL enzyme; (d) a woodchuck hepatitis virus posttranscriptional regulatory element (WPRE); (e) a poly A; and (f) a 3′ ITR.

Various kinds of AAV capsids with broad tissue tropism (the terms “broad tissue tropism” “wide-tropism” are used interchangeably herein) can be used in the rAAV vector described herein. For example, in some embodiments, the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, AAV9 capsid, AAV11, 12, 13, AAVhu.37, AAVrh.8, AAVrh.10, and AAVrh.39, AAV-DJ, or AAV-DJ/8.

Accordingly, in some embodiments, the AAV capsid with wide-tropism is AAV is AAV1. In some embodiments, the AAV capsid with wide-tropism is AAV is AAV2. In some embodiments, the AAV capsid with wide-tropism is AAV is AAV3. In some embodiments, the AAV capsid with wide-tropism is AAV is AAV4. In some embodiments, the AAV capsid with wide-tropism is AAV is AAV5. In some embodiments, the AAV capsid with wide-tropism is AAV is AAV6. In some embodiments, the AAV capsid with wide-tropism is AAV is AAV7. In some embodiments, the AAV capsid with wide-tropism is AAV is AAV8. In some embodiments, the AAV capsid with wide-tropism is AAV is AAV9.

Various kinds of capsids and associated tropism are described in Curr Opin Vir. 2016 Dec. 21:75-80, the contents of which are incorporated herein by reference. By “broad tissue tropism” it is meant that the capsid is able to enable gene transfer to two or more than 2, 3, 4, 5, 6, 7, 8 or more tissue types. For example, in some embodiments, a capsid having broad tissue tropism enable gene transfer to one or more of the following tissues: liver, kidney, heart, gastrointestinal tract, and/or peripheral neurons of the subject.

In some embodiments, the ubiquitous promoter is selected from chicken β actin (CBA) promoter, CAG promoter, EF-1α promoter, PGK promoter, UBC promoter, LSE beta-glucuronidase (GUSB) promoter, or ubiquitous chromatin opening element (UCOE) promoter. In some embodiments, the ubiquitous promoter comprises CBh (CMV enhancer, Chicken beta-actin promoter, Chicken-beta actin-MVM hybrid intron). Accordingly, in some embodiments, the ubiquitous promoter is a chicken β actin (CBA) promoter. In some embodiments, the ubiquitous promoter is an EF-1α promoter. In some embodiments, the EF-1α promoter is in combination with chimeric intron from chicken R-actin and rabbit β-globin genes. In some embodiments, the ubiquitous promoter is a UBC promoter. In some embodiments, the ubiquitous promoter is an LSE beta-glucuronidase (GUSB) promoter. In some embodiments, the ubiquitous promoter is a ubiquitous chromatin opening element (UCOE) promoter. (Powell S K, et al. Discov Med. 2015 January; 19(102):49-57.)

In some embodiments, the ubiquitous promoter comprises a cyto-megalo-virus (CMV) enhancer, chicken beta actin promoter, and a rabbit beta globin intron.

In some embodiments, the ubiquitous promoter comprises a shortened EF-1α promoter and one or more introns.

In some embodiments, the one or more introns are from chicken R-actin and/or rabbit β-globin genes.

In some embodiments, the AAV9 capsid is naturally occurring or modified.

In some embodiments, the WPRE sequence is optional or is modified.

In some embodiments, the WPRE sequence is WPRE mut6delATG.

Exemplary polyA sequences that may be included in the gene therapy vectors encompassed by the present disclosure include human growth hormone polyA (hGHpA), synthetic polyA (SPA), Simian virus 40 late poly A (SV 40 pA) and bovine growth hormone (BGH) poly A. In a particular embodiment, the poly A is bovine growth hormone (BGH) poly A.

In some embodiments, the nucleotide sequence encoding α-GAL enzyme is codon optimized.

In some embodiments, the nucleotide sequence encoding α-GAL enzyme is codon optimized for human cells.

In some embodiments, the α-GAL enzyme has an unmodified sequence.

In some embodiments, the α-GAL enzyme has a modified sequence.

In some embodiments, the nucleotide sequence encoding the α-GAL enzyme is engineered.

In some embodiments, the nucleotide sequence encoding the α-GAL enzyme is engineered and codon optimized.

In some embodiments, the modified sequence comprises one or more amino acid substitutions in comparison to wild-type α-GAL enzyme (SEQ ID NO: 30).

In some embodiments, the modified sequence comprises between 1 and 25 amino acid substitutions in comparison to wild-type α-GAL enzyme (SEQ ID NO: 30). For example, in some embodiments, the modified sequence comprises between 5 and 25 amino acid substitutions in comparison to wild-type α-GAL enzyme (SEQ ID NO: 30). In some embodiments, the modified sequence comprises between 5 and 20 amino acid substitutions in comparison to wild-type α-GAL enzyme (SEQ ID NO: 30). In some embodiments, the modified sequence comprises between 5 and 15 amino acid substitutions in comparison to wild-type α-GAL enzyme (SEQ ID NO: 30). In some embodiments, the modified sequence comprises between 5 and 10 amino acid substitutions in comparison to wild-type α-GAL enzyme (SEQ ID NO: 30). In some embodiments, the modified sequence comprises between 10 and 25 amino acid substitutions in comparison to wild-type α-GAL enzyme (SEQ ID NO: 30). In some embodiments, the modified sequence comprises between 10 and 20 amino acid substitutions in comparison to wild-type α-GAL enzyme (SEQ ID NO: 30). In some embodiments, the modified sequence comprises between 10 and 15 amino acid substitutions in comparison to wild-type α-GAL enzyme (SEQ ID NO: 30).

In some embodiments, the modified sequence comprises between 1 and 10 amino acid substitutions in comparison to wild-type α-GAL enzyme (SEQ ID NO: 30). For example, in some embodiments, the modified sequence comprises between 1 and 9 amino acid substitutions in comparison to wild-type α-GAL enzyme (SEQ ID NO: 30). In some embodiments, the modified sequence comprises between 1 and 8 amino acid substitutions in comparison to wild-type α-GAL enzyme (SEQ ID NO: 30). In some embodiments, the modified sequence comprises between 1 and 7 amino acid substitutions in comparison to wild-type α-GAL enzyme (SEQ ID NO: 30). In some embodiments, the modified sequence comprises between 1 and 6 amino acid substitutions in comparison to wild-type α-GAL enzyme (SEQ ID NO: 30). In some embodiments, the modified sequence comprises between 1 and 5 amino acid substitutions in comparison to wild-type α-GAL enzyme (SEQ ID NO: 30). In some embodiments, the modified sequence comprises between 1 and 4 amino acid substitutions in comparison to wild-type α-GAL enzyme (SEQ ID NO: 30). In some embodiments, the modified sequence comprises between 1 and 3 amino acid substitutions in comparison to wild-type α-GAL enzyme (SEQ ID NO: 30).

In some embodiments, the modified sequence comprises 10 amino acid substitutions in comparison to wild-type α-GAL enzyme (SEQ ID NO: 30).

In some embodiments, a recombinant alpha galactosidase A is provided. In some embodiments, the recombinant alpha galactosidase A comprises a polypeptide sequence having at least 85%, 86%, 87%, 88%, 89%, 90%, 91%, 92%, 93%, 94%, 95%, 96%, 97%, 98%, 99%, or more sequence identity to SEQ ID NO: 30, or a functional fragment thereof. In some embodiments, the recombinant alpha galactosidase A comprises a polypeptide sequence having at least 85% sequence identity to SEQ ID NO: 30. In some embodiments, the recombinant alpha galactosidase A comprises a polypeptide sequence having at least 86% sequence identity to SEQ ID NO: 30. In some embodiments, the recombinant alpha galactosidase A comprises a polypeptide sequence having at least 87% sequence identity to SEQ ID NO: 30. In some embodiments, the recombinant alpha galactosidase A comprises a polypeptide sequence having at least 88% sequence identity to SEQ ID NO: 30. In some embodiments, the recombinant alpha galactosidase A comprises a polypeptide sequence having at least 89% sequence identity to SEQ ID NO: 30. In some embodiments, the recombinant alpha galactosidase A comprises a polypeptide sequence having at least 90% sequence identity to SEQ ID NO: 30. In some embodiments, the recombinant alpha galactosidase A comprises a polypeptide sequence having at least 91% sequence identity to SEQ ID NO: 30. In some embodiments, the recombinant alpha galactosidase A comprises a polypeptide sequence having at least 92% sequence identity to SEQ ID NO: 30. In some embodiments, the recombinant alpha galactosidase A comprises a polypeptide sequence having at least 93% sequence identity to SEQ ID NO: 30. In some embodiments, the recombinant alpha galactosidase A comprises a polypeptide sequence having at least 94% sequence identity to SEQ ID NO: 30. In some embodiments, the recombinant alpha galactosidase A comprises a polypeptide sequence having at least 95% sequence identity to SEQ ID NO: 30. In some embodiments, the recombinant alpha galactosidase A comprises a polypeptide sequence having at least 96% sequence identity to SEQ ID NO: 30. In some embodiments, the recombinant alpha galactosidase A comprises a polypeptide sequence having at least 97% sequence identity to SEQ ID NO: 30. In some embodiments, the recombinant alpha galactosidase A comprises a polypeptide sequence having at least 98% sequence identity to SEQ ID NO: 30. In some embodiments, the recombinant alpha galactosidase A comprises a polypeptide sequence having at least 99% sequence identity to SEQ ID NO: 30.

In some embodiments, the recombinant alpha galactosidase A comprises at least one substitution or substitution in SEQ ID NO:30 at one or more positions selected from: T41/M70/L75/S78/E79/Y123/R193/S197/K237/F248/N247/N278/L286/A292/H302/Q333/K314/L347/M353/S364/A368/S371/K374/K393/F396/E398/W399/R404/M423.

Additional exemplary GLA transgene and α-GAL enzyme sequences can be found in PCT publication nos: PCT/US2021/019811, PCT/US2019/067493 and PCT/US2015/063329, each of which is incorporated by reference herein, in its entirety.

In some embodiments, the modified α-GAL enzyme is selected from one of SEQ ID Nos: 7-17, 33, 34, and 46-60.

In some embodiments, the modified α-GAL enzyme comprises amino acid sequence of SEQ ID NO: 7. In some embodiments, the modified α-GAL enzyme comprises amino acid sequence of SEQ ID NO: 8. In some embodiments, the modified α-GAL enzyme comprises amino acid sequence of SEQ ID NO: 9. In some embodiments, the modified α-GAL enzyme comprises amino acid sequence of SEQ ID NO: 10. In some embodiments, the modified α-GAL enzyme comprises amino acid sequence of SEQ ID NO: 11. In some embodiments, the modified α-GAL enzyme comprises amino acid sequence of SEQ ID NO: 12. In some embodiments, the modified α-GAL enzyme comprises amino acid sequence of SEQ ID NO: 13. In some embodiments, the modified α-GAL enzyme comprises amino acid sequence of SEQ ID NO: 14. In some embodiments, the modified α-GAL enzyme comprises amino acid sequence of SEQ ID NO: 15. In some embodiments, the modified α-GAL enzyme comprises amino acid sequence of SEQ ID NO: 16. In some embodiments, the modified α-GAL enzyme comprises amino acid sequence of SEQ ID NO: 17. In some embodiments, the modified α-GAL enzyme comprises amino acid sequence of SEQ ID NO: 33. In some embodiments, the modified α-GAL enzyme comprises amino acid sequence of SEQ ID NO: 34. In some embodiments, the modified α-GAL enzyme comprises amino acid sequence of SEQ ID NO: 46. In some embodiments, the modified α-GAL enzyme comprises amino acid sequence of SEQ ID NO: 47. In some embodiments, the modified α-GAL enzyme comprises amino acid sequence of SEQ ID NO: 48. In some embodiments, the modified α-GAL enzyme comprises amino acid sequence of SEQ ID NO: 49. In some embodiments, the modified α-GAL enzyme comprises amino acid sequence of SEQ ID NO: 50. In some embodiments, the modified α-GAL enzyme comprises amino acid sequence of SEQ ID NO: 51. In some embodiments, the modified α-GAL enzyme comprises amino acid sequence of SEQ ID NO: 52. In some embodiments, the modified α-GAL enzyme comprises amino acid sequence of SEQ ID NO: 53. In some embodiments, the modified α-GAL enzyme comprises amino acid sequence of SEQ ID NO: 54. In some embodiments, the modified α-GAL enzyme comprises amino acid sequence of SEQ ID NO: 55. In some embodiments, the modified α-GAL enzyme comprises amino acid sequence of SEQ ID NO: 56. In some embodiments, the modified α-GAL enzyme comprises amino acid sequence of SEQ ID NO: 57. In some embodiments, the modified α-GAL enzyme comprises amino acid sequence of SEQ ID NO: 58. In some embodiments, the modified α-GAL enzyme comprises amino acid sequence of SEQ ID NO: 59. In some embodiments, the modified α-GAL enzyme comprises amino acid sequence of SEQ ID NO: 60.

In some embodiments, the modified α-GAL enzyme has increased stability in comparison to wild-type α-GAL enzyme (SEQ ID NO: 30).

In some embodiments, the modified α-GLA enzyme has increased intracellular activity in comparison to wild-type α-GLA enzyme (SEQ ID NO: 30).

In some embodiments, the modified α-GLA enzyme has improved serum stability in comparison to wild-type α-GLA enzyme (SEQ ID NO: 30).

In some embodiments, the modified α-GLA enzyme has improved lysosomal stability in comparison to wild-type α-GLA enzyme (SEQ ID NO: 30).

In some embodiments, the modified α-GLA enzyme has increased specific catalytic activity in comparison to wild-type α-GLA enzyme (SEQ ID NO: 30).

In some aspects, a method of treating Fabry disease in a subject is provided, the method comprising administering to a subject in need thereof a recombinant adeno-associated viral vector (rAAV) as described herein.

In some aspects, a pharmaceutical composition is provided, the composition comprising the rAAV vector as described herein.

In some aspects, a cell is provided, the cell comprising the rAAV vector as described herein. The cell can be any kind of mammalian cell. For example, in some embodiments, the cell is a heart cell. In some embodiments, the cell is a kidney cell. In some embodiments, the cell is a liver. In some embodiments, the cell is a skeletal muscle cell. In some embodiments, the cell is a cell of the gastrointestinal tract. In some embodiments, the cell is a cell of the brain, such as for example a neuron or a glial cell. In some embodiments, the cell is a peripheral neuron.

In some aspects, a method of treating Fabry disease in a subject is provided, the method comprising administering to a subject in need thereof a recombinant adeno-associated viral vector (rAAV) packaged in a rAAV capsid having broad tissue tropism, the vector comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) a poly A; and (e) a 3′ ITR.

In some aspects, a method of treating Fabry disease in a subject is provided, the method comprising administering to a subject in need thereof a recombinant adeno-associated viral vector (rAAV) packaged in a rAAV capsid having broad tissue tropism, the vector comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) a woodchuck hepatitis virus posttranscriptional regulatory element (WPRE); (e) a poly A; and (f) a 3′ ITR.

In some embodiments, the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, or AAV9 capsid. In some embodiments, the wide-tropism AAV capsid is an AAV1 capsid. In some embodiments, the wide-tropism AAV capsid is an AAV2 capsid. In some embodiments, the wide-tropism AAV capsid is an AAV3 capsid. In some embodiments, the wide-tropism AAV capsid is an AAV4 capsid. In some embodiments, the wide-tropism AAV capsid is an AAV5 capsid. In some embodiments, the wide-tropism AAV capsid is an AAV6 capsid. In some embodiments, the wide-tropism AAV capsid is an AAV7 capsid. In some embodiments, the wide-tropism AAV capsid is an AAV8 capsid. In some embodiments, the wide-tropism AAV capsid is an AAV9 capsid.

In some embodiments, the rAAV vector is administered by intravenous, subcutaneous, or transdermal administration. Accordingly, in some embodiments, the rAAV vector is administered intravenously to a subject in need thereof. In some embodiments, the rAAV vector is administered subcutaneously to a subject in need thereof. In some embodiments, the rAAV vector is administered transdermally to a subject in need thereof.

In some embodiments, the transdermal administration is by gene gun.

In some embodiments, the rAAV vector is episomal following administration.

In some embodiments, a rAAV described herein is administered to a subject in need thereof at a dose lower than a dose expected to be used with a AAV vector that targets the liver for expression of α-GAL. In other embodiments, a rAAV described herein, when administered at an equivalent dose as a liver targeted rAAV exhibits higher α-GAL serum and tissue exposure.

In some embodiments, the rAAV vector with broad tissue tropism and using a ubiquitous promoter achieves a therapeutic effect for treating Fabry disease at a lower dose than a rAAV vector comprising an AAV1 capsid, AAV2 capsid, AAV3 capsid, AAV4 capsid, AAV5 capsid, AAV6 capsid, AAV7 capsid, or AAV8 capsid using a liver specific promoter. Accordingly, in some embodiments, the rAAV vector with broad tissue tropism and a ubiquitous promoter achieves a therapeutic effect at a lower dose than a rAAV vector comprising an AAV1 capsid with a liver specific promoter. In some embodiments, the rAAV vector with broad tissue tropism and a ubiquitous promoter achieves a therapeutic effect at a lower dose than a rAAV vector comprising an AAV2 capsid with a liver specific promoter. In some embodiments, the rAAV vector with broad tissue tropism and a ubiquitous promoter achieves a therapeutic effect at a lower dose than a rAAV vector comprising an AAV3 capsid with a liver specific promoter. In some embodiments, the rAAV vector with broad tissue tropism and a ubiquitous promoter achieves a therapeutic effect at a lower dose than a rAAV vector comprising an AAV3 capsid with a liver specific promoter. In some embodiments, the rAAV vector with broad tissue tropism and a ubiquitous promoter achieves a therapeutic effect at a lower dose than a rAAV vector comprising an AAV4 capsid with a liver specific promoter. In some embodiments, the rAAV vector with broad tissue tropism and a ubiquitous promoter achieves a therapeutic effect at a lower dose than a rAAV vector comprising an AAV5 capsid with a liver specific promoter. In some embodiments, the rAAV vector with broad tissue tropism and a ubiquitous promoter achieves a therapeutic effect at a lower dose than a rAAV vector comprising an AAV6 capsid with a liver specific promoter. In some embodiments, the rAAV vector with broad tissue tropism and a ubiquitous promoter achieves a therapeutic effect at a lower dose than a rAAV vector comprising an AAV7 capsid with a liver specific promoter. In some embodiments, the rAAV vector with broad tissue tropism and a ubiquitous promoter achieves a therapeutic effect at a lower dose than a rAAV vector comprising an AAV8 capsid with a liver specific promoter.

In some embodiments, a rAAV vector capable of expressing an α-GAL enzyme may comprise a tissue specific promoter, e.g., a liver specific promoter. Exemplary liver-specific promoters include, but are not limited to, for example, transthyretin promoter (TTR); thyroxine-binding globulin (TBG) promoter; hybrid liver-specific promoter (HLP), and alpha-1-antitrypsin (AAT) promoter.

In some embodiments, following administration of the rAAV vector, the subject has detectable α-GAL in the serum for at least 5 weeks, 10 weeks, 15 weeks, 26 weeks, 1 year, 5 years, 10 years, or 20 years. In some embodiments, following administration of the rAAV vector, the subject has detectable α-GAL in the serum for at least 5 weeks. In some embodiments, following administration of the rAAV vector, the subject has detectable α-GAL in the serum for at least 10 weeks. In some embodiments, following administration of the rAAV vector, the subject has detectable α-GAL in the serum for at least 15 weeks. In some embodiments, following administration of the rAAV vector, the subject has detectable α-GAL in the serum for at least 26 weeks. In some embodiments, following administration of the rAAV vector, the subject has detectable α-GAL in the serum for at least 1 year. In some embodiments, following administration of the rAAV vector, the subject has detectable α-GAL in the serum for at least 5 years. In some embodiments, following administration of the rAAV vector, the subject has detectable α-GAL in the serum for at least 10 years. In some embodiments, following administration of the rAAV vector, the subject has detectable α-GAL in the serum for at least 15 years. In some embodiments, following administration of the rAAV vector, the subject has detectable α-GAL in the serum for at least 20 years. In some embodiments, following administration of the rAAV vector, the subject has detectable α-GAL in the serum for the life of the subject.

In some embodiments, expression of modified α-GAL enzyme provides 3, 10, 30, 100, 300 fold higher serum α-GAL levels compared to the expression of WT α-GAL. In some embodiments, expression of modified α-GAL enzyme provides, 3, 10, 30, 100, 300 fold higher intracellular enzyme levels compared to expression of WT α-GAL.

In some embodiments, the administration results in α-GAL enzyme exposure in one or more of liver, kidney, heart, gastrointestinal tract, brain, and/or peripheral neurons of the subject. Accordingly, in some embodiments, administration results in α-GAL enzyme exposure in the liver. In some embodiments, administration results in α-GAL enzyme exposure in the kidney. In some embodiments, administration results in α-GAL enzyme exposure in the heart. In some embodiments, administration results in α-GAL enzyme exposure in the gastrointestinal tract and cells associated with the gastrointestinal tract. In some embodiments, administration results in α-GAL enzyme exposure in the brain. In some embodiments, administration results in α-GAL enzyme exposure in peripheral neurons.

In some embodiments, administration of rAAV vector results in a survival benefit to Fabry mouse/patient. In some embodiments, administration of the rAAV vector results in reduced levels of globotriaosylceramide (GB3) in one or more of liver, heart, kidney and gastrointestinal tract of the subject. Accordingly, in some embodiments, administration of the rAAV vector results in reduced levels of GB3 in the heart. In some embodiments, administration of the rAAV vector results in reduced levels of GB3 in the skeletal muscle. In some embodiments, administration of the rAAV vector results in reduced levels of GB3 in the kidney. In some embodiments, administration of the rAAV vector results in reduced levels of GB3 in the gastrointestinal tract. Levels of GB3 can be assessed by any means known in the art including for example by chromatographic methods, including for example liquid chromatography-tandem mass spectrometry.

In some aspects, the present disclosure encompasses a method of expressing α-GAL enzyme in a cell, the method comprising administering a rAAV vector packaged in an AAV9 capsid, said vector comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) a bovine growth hormone (BGH) poly A; and (f) a 3′ ITR.

In some aspects, the present disclosure encompasses a method of expressing α-GAL enzyme in a cell, the method comprising administering a rAAV vector packaged in an AAV9 capsid, said vector comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) optionally a woodchuck hepatitis virus posttranscriptional regulatory element (WPRE) having mut6delATG mutation; (e) a bovine growth hormone (BGH) poly A; and (f) a 3′ ITR.

In some aspects, the present disclosure encompasses a recombinant adeno-associated virus (rAAV) vector packaged in an AAV capsid, said vector comprising a 5′ inverted terminal repeat (ITR); a liver specific promoter; a nucleotide sequence encoding α-GAL enzyme; a poly A; and a 3′ ITR.

In some aspects, the present disclosure encompasses a recombinant adeno-associated virus (rAAV) vector packaged in an AAV capsid, said vector comprising a 5′ inverted terminal repeat (ITR); a liver-specific promoter; a nucleotide sequence encoding α-GAL enzyme; a woodchuck hepatitis virus posttranscriptional regulatory element (WPRE); a poly A; and a 3′ ITR.

### BRIEF DESCRIPTION OF FIGURES

FIG. 1 is a vector diagram of exemplary rAAV9 comprising wild-type GAL under the control of a ubiquitous promoter (referred to generally herein as “rAAV9”) as described herein.

FIG. 2 is a graph that shows the level of alpha galactosidase in serum over a period of 12-weeks post injection with rAAV9 in a Fabry (GLAko) mouse model as described herein.

FIG. 3A-FIG. 3M are a series of graphs showing expression of alpha galactosidase in various tissues and reduction of GB3 in various tissues after administration of rAAV9-WT in Fabry GLAko mice. FIG. 3A shows expression of alpha galactosidase in liver, post administration of rAAV9 as compared to rAAV9-null (“null” refers to a AAV9 that does not contain any transgene) and untreated control. FIG. 3B shows expression of alpha galactosidase in kidney, post administration of rAAV9-WT as compared to rAAV9-null and untreated control. FIG. 3C shows expression of alpha galactosidase in heart, post administration of rAAV9-WT as compared to and untreated control. FIG. 3D shows expression of alpha galactosidase in duodenum, post administration of rAAV9-WT as compared to rAAV9-null (null vector) and untreated control. FIG. 3E shows expression of alpha galactosidase in colon, post administration of rAAV9 as compared to rAAV9-null and untreated control. FIG. 3F shows the levels of GB3 in serum as compared to ERT and untreated controls. FIG. 3G shows the levels of GB3 in liver as compared to ERT and untreated controls. FIG. 3H shows the levels of GB3 in kidney as compared to ERT and untreated controls. FIG. 3I shows the levels of GB3 in heart as compared to ERT and untreated controls. FIG. 3J shows the levels of lysoGB3 in serum as compared to ERT and untreated controls. FIG. 3K shows the levels of lysoGB3 in liver as compared to ERT and untreated controls. FIG. 3L shows the levels of lysoGb3 in kidney as compared to ERT and untreated controls. FIG. 3M shows the levels of lysoGb3 in heart as compared to ERT and untreated controls.

FIG. 4A-FIG. 4G are a series of graphs showing expression of alpha galactosidase in various tissues after administration of rAAV9 in severe Fabry-model (G3Stg/GLAko) mice. FIG. 4A shows dose dependent alpha galactosidase activity in serum compared to vehicle (i.e., formulation buffer alone) for 18 weeks. FIG. 4B shows dose dependent increase in alpha galactosidase activity in liver, compared to null vector and vehicle (i.e., formulation buffer alone). FIG. 4C shows dose dependent increase in alpha galactosidase activity in kidney, compared to null vector and vehicle (i.e., formulation buffer alone). FIG. 4D shows dose dependent increase in alpha galactosidase activity in heart, compared to null vector and vehicle (i.e., formulation buffer alone). FIG. 4E shows dose dependent increase in alpha galactosidase activity in duodenum, compared to null vector and vehicle (i.e., formulation buffer alone). FIG. 4F shows dose dependent increase in alpha galactosidase activity in colon, compared to null vector and vehicle (formulation buffer). FIG. 4G shows dose dependent increase in alpha galactosidase activity in brain, compared to null vector and vehicle (i.e., formulation buffer alone).

FIG. 5A-FIG. 5F are a series of graphs showing reduction of GB3 in various tissues after administration of rAAV9 in severe Fabry-model mice. FIG. 5A shows reduction of GB3 in liver after administration of rAAV9-WT as compared to rAAV9-null, and vehicle (i.e., formulation buffer alone). FIG. 5B shows reduction of GB3 in kidney after administration of rAAV9-WT as compared to rAAV9-null, and vehicle (i.e., formulation buffer alone). FIG. 5C shows reduction of GB3 in heart after administration of rAAV9-WT as compared to rAAV9-null, and vehicle (i.e., formulation buffer alone). FIG. 5D shows reduction of GB3 in duodenum after administration of rAAV9-WT as compared to rAAV9-null, and vehicle (i.e., formulation buffer alone). FIG. 5E shows reduction of GB3 in colon after administration of rAAV9-WT as compared to rAAV9-null, and vehicle (i.e., formulation buffer alone). FIG. 5F shows reduction of GB3 in brain after administration of rAAV9-WT as compared to rAAV9-null, and vehicle (i.e., formulation buffer alone). FIG. 5G shows dose-dependent reduction of lysoGb3 in liver as compared to rAAV9-null. FIG. 5H shows dose-dependent reduction of lysoGb3 in kidney as compared to rAAV9-null. FIG. 5I shows dose-dependent reduction of lysoGb3 in heart as compared to rAAV9-null. FIG. 5J shows dose-dependent reduction of lysoGb3 in duodenum as compared to rAAV9-null. FIG. 5K shows dose-dependent reduction of lysoGb3 in colon as compared to rAAV9-null. FIG. 5L shows dose-dependent reduction of lysoGb3 in brain as compared to rAAV9-null.

FIG. 6 is a graph showing increase in body weight after administration of increasing concentrations of rAAV9-WT as compared to null vector (rAAV9-null) and vehicle (i.e., formulation buffer alone) in severe Fabry mice.

FIG. 7A-FIG. 7B are series of graphs showing improvements in kidney function in 27-28 week old severe Fabry-model mice after administration of rAAV9-WT. FIG. 7A shows dose dependent normalization of serum BUN. FIG. 7B shows normalization of urine albumin levels after administration of rAAV9-WT.

FIG. 8A-FIG. 8C are figures showing immunohistochemistry of paw pads of severe Fabry mouse model (G3Stg/GLAko). FIG. 8A shows an immunohistochemistry slide of paw pads of Fabry model mice showing reduction in vacuolation in dorsal root nerves. FIG. 8B shows an immunohistochemistry slide of dose-dependent normalization in PGP9.5 (neuronal marker) staining in Fabry-model mice. FIG. 8C shows an immunohistochemistry slide of dose-dependent increase in MPZ (Schwann cell marker) staining in Fabry-model mice.

FIG. 9A-FIG. 9I show images histopathological features of kidney, heart and dorsal root ganglions (DRG). FIG. 9A shows p62 (autophagy marker) staining observed in a wildtype mouse-kidney collecting ducts. FIG. 9B shows p62 staining observed in a Fabry-untreated mouse-kidney collecting ducts. FIG. 9C shows p62 staining observed in a Fabry mouse-kidney collecting ducts, when treated with 6.25e12 vg/kg of rAAV9-WT FIG. 9D shows p62 staining observed in a wildtype mouse-heart. FIG. 9E shows p62 staining observed in a Fabry-untreated mouse-heart. FIG. 9F shows p62 staining observed in a Fabry mouse-heart, when treated with 6.25e12 vg/kg of rAAV9-WT. FIG. 9G shows CD68 staining observed in a wildtype mouse-DRG. FIG. 9H shows CD68 staining observed in a Fabry-untreated mouse-DRG. FIG. 9I shows CD68 staining observed in a Fabry mouse-DRG, when treated with 6.25e12 vg/kg of rAAV9-WT.

FIG. 10A-10F shows the exposure of α-GAL variants in serum and tissues in Fabry mice 2 days after hydrodynamic tail vein injection of plasmids expressing α-GAL variants. FIG. 10A shows the exposure of α-GAL variants in serum. FIG. 10B shows the exposure of α-GAL variants in kidneys. FIG. 10C shows the exposure of α-GAL variants in heart. FIG. 10D shows the exposure of α-GAL variants in serum. FIG. 10E shows the exposure of α-GAL variants in kidneys. FIG. 10F shows the exposure of α-GAL variants in heart.

FIG. 11A is a graph that shows the level of alpha galactosidase in serum over a period of 12-weeks post injection with either a rAAV9 based construct with a ubiquitous promoter or a rAAV8 based construct with a liver specific promoter. FIG. 11B shows expression of alpha galactosidase in kidney, post administration of rAAV9-WT as compared to rAAV8-WT, as well as to alpha galactosidase protein dosed at 1 mg/kg and an untreated control. FIG. 11C shows percentage reduction of GB3 in kidney after administration of rAAV9-WT as compared to rAAV8-WT, as well as to alpha galactosidase protein dosed at 1 mg/kg and an untreated control.

FIG. 12A-12B are series of graphs showing kidney function in 27-28 week old Fabry-model mice after administration of rAAV9-WT and rAAV8-WT. FIG. 12A shows dose dependent normalization of blood urea nitrogen (BUN) with rAAV9-WT. FIG. 12B shows changes in serum BUN with rAAV8-WT.

FIG. 13A-13E are series of graphs showing serum and tissue galactosidase activities of α-GAL variants after treatment of severe Fabry mice with rAAV9 expressing α-GAL variants. FIG. 13A shows serum alpha galactosidase activity at 5.0×10¹⁰ vg/kg dose from Study 1. FIG. 13B shows serum alpha galactosidase activity at 2.5×10¹¹ dosage according to Study 1. FIG. 13C shows kidney alpha-galactosidase activity at two different dosages according to Study 1. FIG. 13D shows heart alpha-galactosidase activity at two different dosages according to Study 1. FIG. 13E shows liver alpha-galactosidase activity at two different dosages according to Study 1.

FIG. 14A-14D are series of graphs showing serum and tissue alpha-galactosidase levels of α-GAL variants after treatment of severe Fabry mice with rAAV9 expressing α-GAL variants, according to Study 2. FIG. 14A shows serum alpha galactosidase activity at 2.5×10¹¹ vg/kg dose from Study 2. FIG. 14B shows kidney alpha-galactosidase activity at two different dosages according to Study 2. FIG. 14C shows heart alpha-galactosidase activity at two different dosages according to Study 2. FIG. 14D shows liver alpha-galactosidase activity at two different dosages according to Study 2.

FIG. 15A-15D are series of graphs showing presence of GB3 in serum and tissues in mice treated with rAAV9 expressing α-GAL variants, according to Study 1. FIG. 15A shows serum GB3 in response to treatment with rAAV9 expressing α-GAL variants. FIG. 15B shows kidney GB3 in response to treatment with rAAV9 expressing α-GAL variants. FIG. 15C shows heart GB3 in response to treatment with rAAV9 expressing α-GAL variants. FIG. 15D shows liver GB3 in response to treatment with rAAV9 expressing α-GAL variants.

FIG. 16A-16D are series of graphs showing the presence of lysoGb3 in serum and tissues in mice treated with rAAV9 expressing α-GAL variants, according to Study 1. FIG. 16A shows serum lysoGb3 in response to treatment with rAAV9 expressing α-GAL variants. FIG. 16B shows kidney lysoGb3 in response to treatment with rAAV9 expressing α-GAL variants. FIG. 16C shows heart lysoGb3 in response to treatment with variants. FIG. 16D shows liver lysoGb3 in response to treatment with rAAV9 expressing α-GAL variants.

FIG. 17A-17D are series of graphs showing presence of GB3 in serum and tissues in mice treated with rAAV9 expressing α-GAL variants, according to Study 2. FIG. 17A shows serum GB3 in response to treatment with variants. FIG. 17B shows kidney GB3 in response to treatment with rAAV9 expressing α-GAL variants. FIG. 17C shows heart GB3 in response to treatment with rAAV9 expressing α-GAL variants. FIG. 17D shows liver GB3 in response to treatment with rAAV9 expressing α-GAL variants.

FIG. 18A-18D are series of graphs showing the presence of lysoGb3 in serum and tissues in mice treated with rAAV9 expressing α-GAL variants, according to Study 2. FIG. 18A shows serum lysoGb3 in response to treatment with rAAV9 expressing α-GAL variants. FIG. 18B shows kidney lysoGb3 in response to treatment with rAAV9 expressing α-GAL variants. FIG. 18C shows heart lysoGb3 in response to treatment with rAAV9 expressing α-GAL variants. FIG. 18D shows liver lysoGb3 in response to treatment with rAAV9 expressing α-GAL variants.

FIG. 19A-19D are series of graphs showing in vitro α-GAL activity in HUH-7 and HEK293 cells after transfection with plasmids expressing codon optimized α-GAL variants of 004 and D. FIG. 19A is a histogram showing in vitro α-GAL activity in HUH-7 cells transfected with variants of plasmid comprising D, D1-D6, as compared to WT α-GAL and no plasmid. FIG. 19B is a histogram showing in vitro α-GAL activity in HEK293 cells transfected with variants of plasmid comprising D, D1-D6, as compared to WT α-GAL and no plasmid. FIG. 19C is a histogram showing in vitro α-GAL activity in HUH-7 cells transfected with variants of plasmid comprising 004, 004-1 to 004-5, as compared to WT α-GAL and no plasmid. FIG. 19D is a histogram showing in vitro α-GAL activity in HEK293 cells transfected with variants of plasmid comprising 004, 004-1 to 004-5, as compared to WT α-GAL and no plasmid.

FIG. 20A-20D are series of graphs showing dose-dependent α-GAL activity in various tissues in severe Fabry-model mice, treated with rAAV9-D3 and rAAV9-004-3. FIG. 20A is a histogram of dose-dependent α-GAL activity in serum, in severe Fabry-model mice treated with rAAV9-D3 and rAAV9-004-3. FIG. 20B is a histogram of dose-dependent α-GAL activity in kidney, in severe Fabry-model mice treated with rAAV9-D3 and rAAV9-004-3. FIG. 20C is a histogram of dose-dependent α-GAL activity in heart, in severe Fabry-model mice treated with rAAV9-D3 and rAAV9-004-3. FIG. 20D is a histogram of dose-dependent α-GAL activity in liver, in severe Fabry-model mice treated with rAAV9-D3 and rAAV9-004-3.

FIG. 21A-21H are series of graphs showing dose-dependent reduction in substrate in various tissues in severe Fabry-model mice, treated with rAAV9-D3 and rAAV9-004-3. FIG. 21A is a graph showing dose dependent reduction of GB3, in severe Fabry model mice treated with rAAV9-D3 and rAAV9-004-3 in serum. FIG. 21B is a graph showing dose dependent reduction of lysoGb3, in severe Fabry model mice treated with rAAV9-D3 and rAAV9-004-3 in serum. FIG. 21C is a graph showing dose dependent reduction of GB3, in kidney, in severe Fabry model mice treated with rAAV9-D3 and rAAV9-004-3. FIG. 21D is a graph showing dose dependent reduction of lysoGb3 in kidney, in severe Fabry model mice treated with rAAV9-D3 and rAAV9-004-3. FIG. 21E is a graph showing dose dependent reduction of GB3 in heart, in severe Fabry model mice treated with rAAV9-D3 and rAAV9-004-3. FIG. 21F is a graph showing dose dependent reduction of lysoGb3 in heart, in severe Fabry model mice treated with rAAV9-D3 and rAAV9-004-3. FIG. 21G is a graph showing dose dependent reduction of GB3 in liver, in severe Fabry model mice treated with rAAV9-D3 and rAAV9-004-3. FIG. 21H is a graph showing dose dependent reduction of lysoGb3 in liver, in severe Fabry model mice treated with rAAV9-D3 and rAAV9-004-3.

### DEFINITIONS

Approximately or about: As used herein, the term “approximately,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11, 1%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). It is understood that when the term “about” or “approximately” is used to modify a stated reference value, the stated reference value itself is covered along with values that are near the stated reference value on either side of the stated reference value.

Administering: The terms “administer”, “administration” and “administering” refer to providing a composition of the present invention (e.g., a recombinant gene therapy vector expressing alpha galactosidase) to a subject in need thereof (e.g., to a person suffering from the effects of Fabry disease).

Administered in combination: As used herein, the term “administered in combination” or “combined administration” means that two or more agents are administered to a subject at the same time or within an interval such that there can be an overlap of an effect of each agent on the patient. In some embodiments, the administrations of the agents are spaced sufficiently closely together such that a combinatorial (e.g., a synergistic) effect is achieved.

Allogeneic: As used herein, allogeneic refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

Amino acid substitution: The term “amino acid substitution” refers to replacing an amino acid residue present in a parent or reference sequence (e.g., a wild type GLA sequence) with another amino acid residue. An amino acid can be substituted in a parent or reference sequence (e.g., a wild type GLA polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residues.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). In various embodiments described herein, one or more amino acids in the wild-type GLA sequence may be substituted with a different amino acid, thereby resulting in a variant of the α-GAL protein.

Substitutions in a protein or polypeptide amino acid sequence may either be conservative or non-conservative in nature. A conservative amino acid substitution refers to a substitution of a residue with a different residue having a similar side chain, and thus typically involves substitution of the amino acid in a polypeptide (e.g., α-GAL amino acid sequence) with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. A non-conservative substitution refers to substitution of an amino acid in a polypeptide (e.g., α-GAL amino acid sequence) with an amino acid with significantly differing side chain properties. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Blood urea nitrogen: As used herein, the term “Blood urea nitrogen” or “BUN” refers to urea content in blood. Blood urea nitrogen is elevated in pathology associated with kidney. Chronic kidney disease is one of the main features of Fabry disease, causing end-stage renal failure. Gb-3 deposits in glomerular podocytes are thought to contribute at least in part, to the proteinuria or to the rates of progression or severity of kidney involvement in Fabry disease. Blood urea nitrogen measures the efficiency of kidneys to remove urea from the blood. High BUN levels indicate poor renal function.

Chimera: As used herein, “chimera” is an entity having two or more incongruous or heterogeneous parts or regions. For example, a chimeric molecule can comprise a first part comprising a GLA polypeptide, and a second part (e.g., genetically fused to the first part) comprising a second therapeutic protein (e.g., a protein with a distinct enzymatic activity, an antigen binding moiety, or a moiety capable of extending the plasma half-life of α-GAL, for example, an Fc region of an antibody).

Codon substitution: As used herein, the terms “codon substitution” or “codon replacement” in the context of sequence optimization refer to replacing a codon present in a reference nucleic acid sequence with another codon. A codon can be substituted in a reference nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

Codon optimized: The term “codon optimized” or “codon optimization” refers to changes in the codons of the polynucleotide encoding a protein (e.g., GLA gene) such that the encoded protein is more efficiently expressed, e.g., in a cell or an organism. In some embodiments, the polynucleotides encoding the α-GAL enzymes may be codon optimized for optimal production from the host organism(s) and/or cell type(s) selected for expression accounting for GC content, cryptic splice sites, transcription termination signals, motifs that may affect RNA stability, and nucleic acid secondary structures, as well as any other factors of interest.

Engineered variants: The term “engineered α-GAL variants” or “engineered variants” refers to GAL proteins, where, when compared to the wild-type α-GAL, one or more amino acid residues have been modified by substitution, deletion or insertion. In some embodiments, engineered variants are characterized by improved efficacy and pharmacokinetic profiles, due to, for example, modified the structural attributes of the protein. In some embodiments, engineered α-GAL variants enhance clearing of substrates from tissues, such as, serum, kidney, heart and/or liver. The engineered variants may be synthesized or produced recombinantly.

Gb3: As used herein, the term “Gb3” or “globotriaosylceramide” or “GB3” or “gb3” or “CD77” or “GL-3” refers to a type of glycosphingolipid that accumulates in lysosomes in Fabry disease and is considered to be the main causative metabolite.GB3 is formed by α-linkage of galactose to lactosylceramide catalyzed by A4GALT.GB3 is hydrolyzed at the terminal alpha linkage by GLA. Fabry disease is exemplified by accumulation of GB3 in in all organs (especially the heart and kidneys), as well as many cells and urine. Such accumulation is accompanied by a marked increase in the risk of stroke, heart disease (hypertrophic cardiomyopathy, rhythm and conduction system disorders, coronary artery disease, valve abnormalities, etc.) and chronic proteinuria renal failure. In some embodiments, de-acylatedGB3 or lysoGb3 is also a valuable biomarker for Fabry disease.

Gene: The term “gene,” as used herein, refers to a DNA region encoding a protein or polypeptide (e.g., an alpha-galactosidase enzyme, as described herein), as well as all DNA regions which regulate the production of the protein or polypeptide, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

GLA gene: As used herein, the term “GLA gene” or “galactosidase gene” or “alpha-galactosidase gene” or “α-galactosidase gene” refers to a gene which encodes for the enzyme alpha-galactosidase that breaks down globotriaosylceramide. Genetic mutation in the GLA gene results in defective enzyme function of alpha-galactosidase. In humans, the GLA gene is located at Xq22.1, which is the long (q) arm of the X chromosome at position 22.1. Some of the other names by which the GLA gene may be referred to include AGAL HUMAN, Agalsidase alpha, Alpha-D-galactosidase A, alpha-D-galactosidase galactohydrolase, Alpha-galactosidase, alpha-Galactosidase A, ceramidetrihexosidase, GALA, galactosidase or Melibiase.

Galactosidase: The term “galactosidase” or “alpha galactosidase A” or “α-galactosidase A” or “α-GAL”, as used herein, refers to the enzyme encoded by the GLA gene. Human alpha galactosidase (EC 3.2.1.22) is a lysosomal enzyme which hydrolyses terminal alpha galactosyl moieties from glycolipids and glycoproteins. As used herein, the term α-GAL may refer to wild-type enzyme or a variant thereof. Deficiency of alpha galactosidase A causes Fabry disease (also referred to as angiokeratoma corporis diffusum, Anderson-Fabry disease, hereditary dystopic lipidosis, alpha-galactosidase A deficiency, α-GAL deficiency, and ceramide trihexosidase deficiency), which is an X-linked inborn error of glycosphingolipid catabolism. In various embodiments described herein, a gene therapy platform is provided for treatment of Fabry disease.

“Improved enzyme property”: The term “improved enzyme property” refers to any property or attribute of an engineered α-GAL polypeptide that is an improvement relative to the same property or attribute of a reference α-GAL polypeptide (e.g., as compared to a wild-type α-GAL polypeptide or another engineered α-GAL polypeptide). Improved properties include, but are not limited to such properties as increased gene expression, increased protein production, increased thermoactivity, increased thermostability, increased activity at various pH levels, increased stability, increased enzymatic activity, increased substrate specificity or affinity, increased specific activity, increased resistance to substrate and/or product inhibition, increased chemical stability, improved chemoselectivity, improved solvent stability, increased tolerance to acidic, neutral, or basic pH, increased tolerance to proteolytic activity (i.e., reduced sensitivity to proteolysis), reduced aggregation, increased solubility, reduced immunogenicity, improved post-translational modification (e.g., glycosylation), altered temperature profile, increased cellular uptake, increased lysosomal stability, increased ability to deplete cells of GB3, increased secretion from α-GAL producing cells, etc. In various embodiments, the gene therapy vectors encompassed by the present disclosure comprise a nucleic acid sequence encoding an α-GAL polypeptide comprising one or more improved enzyme properties relative to a reference α-GAL polypeptide. In some embodiments, the nucleic acid sequence encoding an α-GAL polypeptide exhibiting one or more improved enzyme properties, is codon optimized.

In various embodiments, codon optimized and/or engineered α-GAL variants exhibit one or more aforementioned improved properties. In a particular embodiment, α-GAL variant having amino acid sequence set forth in SEQ ID NO: 10 or SEQ ID NO: 50 has improved serum and lysosomal stability and α-GAL variant having amino acid sequence set forth in SEQ ID NO: 14 or SEQ ID NO: 55 has increased specific catalytic activity over wild type α-GAL polypeptide.

“Increased enzymatic activity”: The term “increased enzymatic activity” refers to an increase in specific activity (e.g., product produced/time/weight protein) or an increase in percent conversion of the substrate to the product (e.g., percent conversion of starting amount of substrate to product in a specified time period) using a specified amount of an engineered α-GAL enzyme as compared to a reference α-GAL enzyme (e.g., a wild type α-GAL enzyme or another engineered variant). Any suitable method known in the art and/or those described herein may be used to determine enzyme activity. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than a reference α-GAL enzyme.

Intrinsic Expression: The term “intrinsic expression” and grammatical equivalents thereof refers to expression of a gene within one or more cells into which a transgene is introduced. Intrinsic expression uses the cell's own or pre-existing transcription or translation mechanisms and resources for expression of the transgene. For example, in some embodiments, when this term is used to refer to an “intrinsic α-GAL expression system” it means that α-GAL is expressed from within the cells of a tissue.

Nucleic acid: As used herein, the terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

Operative linkage: As used herein, the terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

Physiological pH: As used herein, “physiological pH” means the pH range generally found in a subject's (e.g., human) blood.

Basic pH: The term “basic pH” (e.g., used with reference to improved stability at basic pH conditions or increased tolerance to basic pH) means a pH range of about 7 to 11.

Acidic pH: The term “acidic pH” (e.g., used with reference to improved stability to acidic pH conditions or increased tolerance to acidic pH) means a pH range of about 1.5 to 4.5. Polypeptide: As used herein, the terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

Promoter: As used herein, the term “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. In some embodiments, liver-specific promoters include, for example, transthyretin promoter (TTR); thyroxine-binding globulin (TBG) promoter; hybrid liver-specific promoter (HLP), and alpha-1-antitrypsin (AAT) promoter. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

Sequence Optimization: As used herein, the term “sequence optimization” refers to a process or series of processes by which nucleobases in a reference nucleic acid sequence are replaced with alternative nucleobases, resulting in a nucleic acid sequence with improved properties, e.g., improved protein expression or increased activity.

Tropism: As used herein, the terms “tropism,” or “tropicity” in the context of AAV refers to AAV capsid serotype having varying transduction profiles for different tissue types. In some embodiments, “systemic tropism” and “systemic transduction” (and equivalent terms) indicate that the virus capsid or virus vector of the invention exhibits tropism for or transduces, respectively, more than one tissue, or multiple tissues or organs throughout the body (e.g., more than one of brain, lung, skeletal muscle, heart, liver, kidney and/or pancreas).

Vector: As used herein, the term “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. In some embodiments, the vector is a virus, which includes, for example, encapsulated forms of vector nucleic acids, and viral particles in which the vector nucleic acids have been packaged. In some embodiments, the vector is not a wild-type strain of a virus, in as much as it comprises human-made mutations or modifications. In some embodiments, the vector is derived from a wild-type viral strain by genetic manipulation (i.e., by deletion) to comprise a conditionally replicating virus, as further described herein. In some embodiments, the vector is delivered by non-viral means. In some embodiments, vectors described herein are gene therapy vectors, which are used as carriers for delivery of polynucleotide sequences (e.g., an alpha galactosidase enzyme) to cells. In a particular embodiment, a gene therapy vector described herein is a recombinant AAV vector (e.g., AAV8 or AAV9).

Wild-type: As used herein, the term “wild-type” and “naturally-occurring” refer to the form of a nucleic acid or protein found in nature. For example, a wild-type polypeptide or polynucleotide sequence is a sequence present in an organism that can be isolated from a source in nature and which has not been intentionally modified by human manipulation.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### DETAILED DESCRIPTION

Fabry disease is an X-linked inherited disease that results in the production of aberrant lysosomal hydrolase, α-galactosidase A (α-GAL), due to mutations in the GLA gene. Because α-GAL is necessary for catabolism of glycolipids, such as sphingolipids, deficiency or malfunction of α-GAL causes accumulation of sphingolipids in tissues. Fabry diseases affects 1 in 40,000 males, who develop multisystem disease develops in childhood or adolescence. Clinical manifestations of Fabry disease include, but are not limited to burning, tingling, or pricking sensations or numbness in the extremities, heat intolerance, skin lesions called angiokeratomas, corneal opacities, cardiac arrhythmias, left ventricular hypertrophy, proteinuria, renal insufficiency and cerebrovascular accidents such as stroke and/or seizure. Heterozygous females for the GLA gene can transmit the disease to their sons and are usually free of symptoms. However, some females develop corneal opacity or more severe manifestations due to uneven X chromosome inactivation.

Current treatment options for Fabry disease include recombinant enzyme replacement therapies (ERTs). ERTs slow the progression of the Fabry disease but do not completely halt or reverse the disease. Current treatment for Fabry disease predominantly achieves a slowing of disease progression limited to kidney and heart, with inadequate or no improvements in other organs/tissues. Fabry patients also require continuous protein-based infusion, sometimes resulting in infusion reactions and augmented immunogenicity. Such continuous disease management requirements also increase the “treatment burden” or the added and ongoing workload (i.e. necessities and demands) for patients in order for them to adhere to recommendations made by their clinicians to manage their morbidity and wellbeing. In severely affected classical male Fabry disease patients, yearly loss of renal function despite treatment is up to −6.82 mL/min/1.73 m²/year (Germain et al, J Med Genet. 2015 May; 52(5):353-8.2015), as compared to healthy subjects, who have a yearly loss of renal function of −1 mL/min/1.73 m²/year. These needs could be addressed by vector delivery of GLA as described herein.

One advantage of a gene therapy approach to the treatment of Fabry disease is continuous α-GAL exposure rather than an intermittent α-GAL exposure provided by ERT infusion. The gene therapy approach can potentially allow for uptake by certain tissues and cell types (e.g., cardiomyocytes, peripheral neurons and kidney podocytes) that is not easily achieved by infused ERTs. The constant availability of α-GAL in the lysosomes can prevent glycosphingolipid re-accumulation between doses. The significant enhancement in enzyme distribution to target cells could provide a transformative therapy with the possibility of achieving superior clinical benefit over current therapies. In addition, gene therapy accompanied by hepatocyte transduction can harness the tolerogenic nature of the liver and induce systemic immunological tolerance to transgene product eliminating the risk of reduced treatment efficacy due to anti-drug antibodies. Without wishing to be bound by theory, it is believed that these benefits, combined with a single long-lasting dose, could both address the need for a treatment with a significantly higher treatment efficacy and reduce treatment burden on patients and caregivers.

The present disclosure provides, among other things, (1) an intrinsic GLA expression system in tissues affected by Fabry disease, (2) methods to achieve sustained and high expression of α-GAL to reduce disease burden and treatment burden associated with progression of Fabry disease and (3) use of a vector encoding GLA to achieve reduction in Fabry disease associated phenotypes.

In some embodiments, an intrinsic GLA expression system as provided herein comprises a viral vector, comprising a sequence encoding α-GAL, controlled by a ubiquitous promoter. In some embodiments, the promoter is a mammalian ubiquitous promoter. In some embodiments, it is believed that ubiquitous promoter achieves broad distribution of encoded α-GAL in a mammal. Current gene therapy approaches to treat Fabry disease mostly rely on the use of liver-specific promoters, unlike the delivery vehicles described herein. The gene therapy systems for Fabry disease that use a liver-specific promoter relies on α-GAL production from the liver and “cross-correction” of other tissues. This disclosure provides a system for intrinsic expression of α-GAL in multiple target tissues resulting in broader exposure of α-GAL and better treatment of Fabry disease and the associated symptoms using a gene therapy system using a ubiquitous promoter. As provided in more detail below, the ubiquitous promoter as used in this disclosure can be selected from one or more of EF-1α promoter, UBC promoter, LSE beta-glucuronidase (GUSB) promoter, ubiquitous chromatin opening element (UCOE) promoter, GAPDH promoter, chicken β actin (CBA) promoter, PGK promoter and mini EF1 promoter. In some embodiments, the ubiquitous promoter can be engineered from one of more known ubiquitous promoters.

This disclosure provides a system for broader exposure of α-GAL and a more effective treatment of Fabry disease and the associated symptoms using a gene therapy system which employs a ubiquitous promoter. As provided in more detail below, the ubiquitous promoter as used in this disclosure can be selected from one or more of EF-1α promoter, UBC promoter, LSE beta-glucuronidase (GUSB) promoter, ubiquitous chromatin opening element (UCOE) promoter, GAPDH promoter, chicken β actin (CBA) promoter, PGK promoter and mini EF1 promoter. In some embodiments, the ubiquitous can be engineered from one of more known ubiquitous promoters.

In some embodiments, an intrinsic GLA expression system as provided herein comprises a viral vector that can improve the exposure or distribution of α-GAL in various tissues in a mammal. In some embodiments, the improved exposure or distribution of α-GAL in various tissues improves the symptoms associated with Fabry disease. In some embodiments, the use of a viral vector complements the use of ubiquitous promoter in providing wider tropism of GLA. It is generally anticipated that wider tropism of GLA will improve the symptoms of Fabry disease. In some embodiments, the viral vector is selected from one or more of AAV1, AAV2, AAV3, AAV4, AAV5, AAV6, AAV7, AAV8 or AAV9 having a ubiquitous promoter. In some embodiments, an appropriate viral vector with wide tropism can be engineered with combined elements of AAV1, AAV2, AAV3, AAV4, AAV5, AAV6, AAV7, AAV8 or AAV9 having a ubiquitous promoter.

In some embodiments, a viral vector encompassed by the present disclosure comprises a tissue specific promoter, e.g., a liver specific promoter upstream of a nucleic acid sequence encoding an α-GAL polypeptide.

In some embodiments, an intrinsic GLA expression system as provided herein comprises a viral vector that can improve the exposure or distribution of α-GAL in various tissues in a mammal. In some embodiments, the improved exposure or distribution of α-GAL in various tissues improves the symptoms associated with Fabry disease. In some embodiments, the use of a viral vector complements the use of ubiquitous promoter in providing robust tissue distribution of α-GAL. It is generally anticipated that improved biodistribution of α-GAL will improve the symptoms of Fabry disease. In some embodiments, the viral vector is selected from one or more of AAV1, AAV2, AAV3, AAV4, AAV5, AAV6, AAV7, AAV8 or AAV9 having a ubiquitous promoter. In some embodiments, an appropriate viral vector with wide tropism can be engineered with combined elements of AAV1, AAV2, AAV3, AAV4, AAV5, AAV6, AAV7, AAV8 or AAV9 having a ubiquitous promoter.

In some embodiments, an intrinsic GLA expression system as provided herein is a viral vector expressing a wild type or a variant α-GAL (e.g., an engineered variant). In some embodiments, the instant disclosure provides a viral vector system comprising a ubiquitous promoter for broad, tissue-wide distribution of wild-type α-GAL. In some embodiments, the instant disclosure provides a viral vector system comprising a ubiquitous promoter for broad, tissue-wide distribution of a α-GAL variant.

In some embodiments, a GLA transgene encodes an enzyme with improved the serum or tissue stability of α-GAL, compared to wild-type α-GAL. In some embodiments, a GLA transgene encodes an enzyme with higher α-GAL activity compared to wild type enzyme. In some embodiments, a GLA transgene described herein encodes an α-GAL enzyme comprising one or more amino acid modifications at positions 1 to 100 of wild-type α-GAL. In some embodiments, a α-GAL enzyme encoded by a GLA transgene comprises one or more amino acid modifications at positions 101-200 of wild-type α-GAL. In some embodiments, a α-GAL enzyme encoded by a GLA transgene comprises one or more amino acid modifications at positions 201-300 of wild-type α-GAL. In some embodiments, a α-GAL enzyme encoded by a GLA transgene comprises one or more amino acid modifications at positions 301-400 of wild-type α-GAL. In some embodiments, a α-GAL enzyme encoded by a GLA transgene comprises one or more amino acid modifications at positions 401-429 of wild-type α-GAL. In some embodiments, the modification can be an amino acid substitution. In some embodiments, the modification can be an amino acid deletion. In some embodiments, the modification can be an amino acid insertion. In some embodiments, the amino acid substitution can be a conservative substitution. In some embodiments, the amino acid substitution can be a non-conservative substitution. In some embodiments, the α-GAL encoded by a GLA transgene comprises an amino acid sequence selected from SEQ ID NO: 7-17, 33 or 34.

### Gene Therapy Vectors

The vectors described herein comprise a GLA sequence. In some embodiments, the GLA sequence can be naturally occurring (wild-type) sequence (SEQ ID NO: 30). In some embodiments, the GLA sequence can be a modified sequence, for example a codon-optimized GLA sequence or an engineered or modified GLA sequence. Exemplary GLA sequences contemplated for use in the vectors of the present disclosure are provided in the Table 1 below.

In some embodiments, an α-GAL encoded by a GLA transgene comprises an a signal peptide sequence MQLRNPELHLGCALALRFLALVSWDIPGARA (SEQ ID NO: 76). In some embodiments, an α-GAL encoded by a GLA transgene comprises an a signal peptide sequence at the N-terminus. In some embodiments, an α-GAL encoded by a GLA transgene comprises an a signal peptide sequence at the C-terminus. In some embodiments, an α-GAL encoded by a GLA transgene comprises SEQ ID NO: 76 at the N-terminus. In some embodiments, an α-GAL encoded by a GLA transgene comprises SEQ ID NO: 76 at the N-terminus.

In some embodiments, a GLA sequence comprises a signal peptide sequence atgcagctgaggaacccagaactacatctgggctgcgcgcttgcgcttcgcttcctggccctcgtttcctgggacatccctggggctagagc a (SEQ ID NO: 77). In some embodiments, a GLA sequence comprises a signal peptide sequence at the 5′ end. In some embodiments, a GLA sequence comprises a signal peptide sequence at the 3′ end. In some embodiments, a GLA sequence comprises SEQ ID NO: 77 at the 5′ end. In some embodiments, a GLA sequence comprises SEQ ID NO: 77 at the 3′ end.

TABLE 1
Exemplary α-GAL Amino Acid and GLA Transgene Nucleotide Sequences

| | |
|---|---|
| Identifier | Sequence |

Exemplary α-GAL Amino Acid and GLA Transgene Nucleotide Sequences

| | |
|---|---|
| A | LDNGLARTPTMGWLHWERFMCNLDCQEEPDSCISEKLFMEMAERMVSEGWKDAGY |
| | EYLCIDDCWMAPQRDSEGRLQADPQRFPHGIRQLANYVHSKGLKLGIYADVGNKT |
| | CAGFPGSFGYYDIDAQTFADWGVDLLKFDGCYCDSLENLADGYKHMSLALNRTGR |
| | PIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAAPLFMSNDLRHISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRQLGFYEWTSRLKSHINPTGTVLLQ |
| | LENTMQMSLKDLL (SEQ ID NO: 7) |
| B | LDNGLARTPPMGWLHWERFMCNLDCQEEPDSCISEKLFEEMAERMVTEGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNRTGR |
| | PIVYSCEWPLYMWPFQKPNYTEIRQYCNHWRNFADIDDSWASIKSILDWTSRNQE |
| | RIVDVAGPGGWNDPDMLVIGNEGLSWDQQVTQMALWAIMAGPLFMSNDLRAISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRKLGFYEATSRLRSHINPTGTVLLQ |
| | LENTMQTSLKDLL (SEQ ID NO: 8) |
| C | LDNGLARTPPMGWLHWERFMCNLDCQEEPDSCISEKLFEEMAERMVTEGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNKTGR |
| | PIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAGPLEMSNDLRAISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRKLGFYEATSRLRSHINPTGTVLLQ |
| | LENTMQTSLKDLL (SEQ ID NO: 9) |
| D | LDNGLARTPPMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNKTGR |
| | PIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAGPLFMSNDLRAISPQ |
| | AKALLQDKDVIAINQDPLGKQGYQLRKGDNFEVWERPLSGDAWAVAIINRQEIGG |
| | PRGYTIPVASLGKGVACNPACFITQLLPVKRKLGFYEATSRLRSHINPTGTVLLQ |
| | LENTMQTSLKDLL (SEQ ID NO: 10) |
| E | LDNGLARTPPMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNKTGR |
| | PIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAGPLFMSNDLRAISPQ |
| | AKALLQDKDVIAINQDPLGKQGYQLRKGDNFEVWERPLSGDAWAVAIINRQEIGG |
| | PRGYTIPVAKLGKGVACNPACFITQLLPVKRKLGFYEATSRLRSHINPTGTVLLQ |
| | LENTMQTSLKDLL (SEQ ID NO: 11) |
| F | LDNGLARTPPMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNKTGR |
| | DIVYSCEWPLYMWPFQKPNYTEIRQYCNHWRNFADIDDSWASIKSILDWTSRNQE |
| | RIVDVAGPGGWNDPDMLVIGNEGLSWDQQVTQMALWAIMAGPLFMSNDLRAISPQ |
| | AKALLQDTDVIAINQDPLGKQGYQLRKGDNFEVWERPLSGDAWAVAIINRQEIGG |
| | PRGYTIPVAKLGKGVACNPACFITQLLPVKRKLGFYEATSRLRSHINPTGTVLLQ |
| | LENTMQTSLKDLL (SEQ ID NO: 46) |
| 002 | LDNGLARTPTMGWLHWERFMCNLDCQEEPDSCISEKLEMEMAELMVSEGWKDAGY |
| | EYLCIDDCWMAPQRDSEGRLQADPQRFPHGIRQLANYVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWASIKSILDWTSFNQE |
| | RIVDVAGPGGWNDPDMLVIGNFGLSWDQQVTQMALWAIMAAPLFMSNDLRHISPQ |
| | AKALLQDKDVIAINQDPLGKQGYQLRQGDNFEVWERPLSGLAWAVAVINRQEIGG |
| | PRSYTIAVASLGGGVACNPACFITQLLPVKRKLGLYEWTSRLKSHINPTGTVLLQ |
| | LENTMQMSLKDLL (SEQ ID NO: 12) |
| 003 | LDNGLARTPTMGWLHWERFMCNLDCQEEPDSCISEKLFMEMAELMVSEGWKDAGY |
| | EYLCIDDCWMAPQRDSEGRLQADPQRFPHGIRQLANYVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWKSIKSILDWTSENQE |
| | RIVDVAGPGGWNDPDMLVIGNFGLSWNQQVTQMALWAIMAAPLFMSNDLRHISPQ |
| | AKALLQDKDVIAINQDPLGKQGYQLRKGDNFEVWERPLSGDAWAVAMINRQEIGG |
| | PRSYTIPVASLGKGVACNPACFITQLLPVKRKLGFYEWTSRLRSHINPTGTVLLQ |
| | LENTMQMSLKDLL (SEQ ID NO: 13) |
| 004 | LDNGLARTPTMGWLHWERFMCNLDCQEEPDSCISEKLEMEMAERMVSEGWKDAGY |
| | EYLCIDDCWMAPQRDSEGRLQADPQRFPHGIRQLANYVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWASIKSILDWTSRNQE |
| | RIVDVAGPGGWNDPDMLVIGNFGLSWDQQVTQMASWAIMAAPLFMSNDLRHISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRQLGFYNWTSRLKSHINPTGTVLLQ |
| | LENTMQMSLKDLL (SEQ ID NO: 14) |
| 005 | LDNGLARTPTMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWKSIKSILDWTSRNQE |
| | RIVDVAGPGGWNDPDMLVIGNFGLSWNQQVTQMALWAIMAAPLFMSNDLRHISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRKLGFYEWTSRLRSHINPTGTVLLQ |
| | LENTMQMSLKDLL (SEQ ID NO: 15) |
| 006 | LDNGLARTPTMGWLHWERFMCNLDCQEEPDSCISEKLFEEMAELMVSEGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWKSIKSILDWTSFNQE |
| | RIVDVAGPGGWNDPDMLVIGNEGLSWNQQVTQMALWAIMAAPLFMSNDLRHISPQ |
| | AKALLQDKDVIAINQDPLGKQGYQLRQGDNFEVWERPLSGDAWAVAMINRQEIGG |
| | PRSYTIPVASLGKGVACNPACFITQLLPVKRKLGFYEWTSRLRSHINPTGTVLLQ |
| | LENTMQMSLKDLL (SEQ ID NO: 16) |
| 007 | LDNGLARTPTMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWKSIKSILDWTSRNQE |
| | RIVDVAGPGGWNDPDMLVIGNFGLSWDQQVTQMALWAIMAAPLFMSNDLRHISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRKLGFYEATSRLKSHINPTGTVLLQ |
| | LENTMQMSLKDLL (SEQ ID NO: 17) |
| 008 | LDNGLARTPTMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAAPLFMSNDLRHISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRQLGFYNWTSRLKSHINPTGTVLLQ |
| | LENTMQMSLKDLL (SEQ ID NO: 33) |
| 009 | LDNGLARTPPMGWLHWERFMCNLDCQEEPDSCISEKLFEEMAERMVTDGWKDAGY |
| | EYLCIDDCWMAPQRDSEGRLQADPQRFPHGIRQLANHVHSKGLKLGIYADVGNKT |
| | CAGFPGSFGYYDIDAQTFADWGVDLLKFDGCYCDSLENLADGYKHMSLALNKTGR |
| | PIVYSCEWPLYMWPFQKPNYTEIRQYCNHWRNFADIDDSWASIKSILDWTSRNQE |
| | RIVDVAGPGGWNDPDMLVIGNFGLSWDQQVTQMALWAIMAAPLFMSNDLRHISPQ |
| | AKALLQDKDVIAINQDPLGKQGYQLRKGDNFEVWERPLSGDAWAVAIINRQEIGG |
| | PRSYTIPVASLGKGVACNPACFITQLLPVKRQLGFYNWTSRLKSHINPTGTVLLQ |
| | LENTMQMSLKDLL (SEQ ID NO: 34) |
| WT | LDNGLARTPTMGWLHWERFMCNLDCQEEPDSCISEKLEMEMAELMVSEGWKDAGY |
| | EYLCIDDCWMAPQRDSEGRLQADPQRFPHGIRQLANYVHSKGLKLGIYADVGNKT |
| | CAGFPGSFGYYDIDAQTFADWGVDLLKFDGCYCDSLENLADGYKHMSLALNRTGR |
| | SIVYSCEWPLYMWPFQKPNYTEIRQYCNHWRNFADIDDSWKSIKSILDWTSFNQE |
| | RIVDVAGPGGWNDPDMLVIGNFGLSWNQQVTQMALWAIMAAPLFMSNDLRHISPQ |
| | AKALLQDKDVIAINQDPLGKQGYQLRQGDNFEVWERPLSGLAWAVAMINRQEIGG |
| | PRSYTIAVASLGKGVACNPACFITQLLPVKRKLGFYEWTSRLRSHINPTGTVLLQ |
| | LENTMQMSLKDLL (SEQ ID NO: 30) |

Exemplary α-GLA Amino Acid with Signal Peptide

| | |
|---|---|
| A | MQLRNPELHLGCALALRFLALVSWDIPGARALDNGLARTPTMGWLHWERFMCNLD |
| | CQEEPDSCISEKLFMEMAERMVSEGWKDAGYEYLCIDDCWMAPQRDSEGRLQADP |
| | QRFPHGIRQLANYVHSKGLKLGIYADVGNKTCAGFPGSFGYYDIDAQTFADWGVD |
| | LLKFDGCYCDSLENLADGYKHMSLALNRTGRPIVYSCEWPLYMWPFQKPNYTEIR |
| | QYCNHWRNFADIDDSWASIKSILDWTSRNQERIVDVAGPGGWNDPDMLVIGNFGL |
| | SWDQQVTQMALWAIMAAPLFMSNDLRHISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRQLGFYEWTSRLKSHINPTGTVLLQLENTMQMSLKDLL (SEQ ID NO: |
| | 47) |
| B | MQLRNPELHLGCALALRFLALVSWDIPGARALDNGLARTPPMGWLHWERFMCNLD |
| | CQEEPDSCISEKLFEEMAERMVTEGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNRTGRPIVYSCEWPLYMWPFQKPNYTEIR |
| | QYCNHWRNFADIDDSWASIKSILDWTSRNQERIVDVAGPGGWNDPDMLVIGNEGL |
| | SWDQQVTQMALWAIMAGPLFMSNDLRAISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRKLGFYEATSRLRSHINPTGTVLLQLENTMQTSLKDLL (SEQ ID NO: |
| | 48) |
| C | MQLRNPELHLGCALALRFLALVSWDIPGARALDNGLARTPPMGWLHWERFMCNLD |
| | CQEEPDSCISEKLFEEMAERMVTEGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNKTGRPIVYSCEWPLYMWPFQKPNYTEIR |
| | QYCNHWRNFADIDDSWASIKSILDWTSRNQERIVDVAGPGGWNDPDMLVIGNFGL |
| | SWDQQVTQMALWAIMAGPLFMSNDLRAISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRKLGFYEATSRLRSHINPTGTVLLQLENTMQTSLKDLL (SEQ ID NO: |
| | 49) |
| D | MQLRNPELHLGCALALRFLALVSWDIPGARALDNGLARTPPMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNKTGRPIVYSCEWPLYMWPFQKPNYTEIR |
| | QYCNHWRNFADIDDSWASIKSILDWTSRNQERIVDVAGPGGWNDPDMLVIGNFGL |
| | SWDQQVTQMALWAIMAGPLFMSNDLRAISPQAKALLQDKDVIAINQDPLGKQGYQ |
| | LRKGDNFEVWERPLSGDAWAVAIINRQEIGGPRGYTIPVASLGKGVACNPACFIT |
| | QLLPVKRKLGFYEATSRLRSHINPTGTVLLQLENTMQTSLKDLL (SEQ ID NO: |
| | 50) |
| E | MQLRNPELHLGCALALRFLALVSWDIPGARALDNGLARTPPMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNKTGRPIVYSCEWPLYMWPFQKPNYTEIR |
| | QYCNHWRNFADIDDSWASIKSILDWTSRNQERIVDVAGPGGWNDPDMLVIGNFGL |
| | SWDQQVTQMALWAIMAGPLFMSNDLRAISPQAKALLQDKDVIAINQDPLGKQGYQ |
| | LRKGDNFEVWERPLSGDAWAVAIINRQEIGGPRGYTIPVAKLGKGVACNPACFIT |
| | QLLPVKRKLGFYEATSRLRSHINPTGTVLLQLENTMQTSLKDLL (SEQ ID NO: |
| | 51) |
| F | MQLRNPELHLGCALALRFLALVSWDIPGARALDNGLARTPPMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNKTGRDIVYSCEWPLYMWPFQKPNYTEIR |
| | QYCNHWRNFADIDDSWASIKSILDWTSRNQERIVDVAGPGGWNDPDMLVIGNEGL |
| | SWDQQVTQMALWAIMAGPLFMSNDLRAISPQAKALLQDTDVIAINQDPLGKQGYQ |
| | LRKGDNFEVWERPLSGDAWAVAIINRQEIGGPRGYTIPVAKLGKGVACNPACFIT |
| | QLLPVKRKLGFYEATSRLRSHINPTGTVLLQLENTMQTSLKDLL (SEQ ID NO: |
| | 52) |
| 002 | MQLRNPELHLGCALALRFLALVSWDIPGARALDNGLARTPTMGWLHWERFMCNLD |
| | CQEEPDSCISEKLFMEMAELMVSEGWKDAGYEYLCIDDCWMAPQRDSEGRLQADP |
| | QRFPHGIRQLANYVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWASIKSILDWTSFNQERIVDVAGPGGWNDPDMLVIGNEGL |
| | SWDQQVTQMALWAIMAAPLFMSNDLRHISPQAKALLQDKDVIAINQDPLGKQGYQ |
| | LRQGDNFEVWERPLSGLAWAVAVINRQEIGGPRSYTIAVASLGGGVACNPACFIT |
| | QLLPVKRKLGLYEWTSRLKSHINPTGTVLLQLENTMQMSLKDLL (SEQ ID NO: |
| | 53) |
| 003 | MQLRNPELHLGCALALRFLALVSWDIPGARALDNGLARTPTMGWLHWERFMCNLD |
| | CQEEPDSCISEKLFMEMAELMVSEGWKDAGYEYLCIDDCWMAPQRDSEGRLQADP |
| | QRFPHGIRQLANYVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWKSIKSILDWTSFNQERIVDVAGPGGWNDPDMLVIGNEGL |
| | SWNQQVTQMALWAIMAAPLFMSNDLRHISPQAKALLQDKDVIAINQDPLGKQGYQ |
| | LRKGDNFEVWERPLSGDAWAVAMINRQEIGGPRSYTIPVASLGKGVACNPACFIT |
| | QLLPVKRKLGFYEWTSRLRSHINPTGTVLLQLENTMQMSLKDLL (SEQ ID NO: |
| | 54) |
| 004 | MQLRNPELHLGCALALRFLALVSWDIPGARALDNGLARTPTMGWLHWERFMCNLD |
| | CQEEPDSCISEKLFMEMAERMVSEGWKDAGYEYLCIDDCWMAPQRDSEGRLQADP |
| | QRFPHGIRQLANYVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWASIKSILDWTSRNQERIVDVAGPGGWNDPDMLVIGNEGL |
| | SWDQQVTQMASWAIMAAPLFMSNDLRHISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRQLGFYNWTSRLKSHINPTGTVLLQLENTMQMSLKDLL (SEQ ID NO: |
| | 55) |
| 005 | MQLRNPELHLGCALALRFLALVSWDIPGARALDNGLARTPTMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWKSIKSILDWTSRNQERIVDVAGPGGWNDPDMLVIGNEGL |
| | SWNQQVTQMALWAIMAAPLFMSNDLRHISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRKLGFYEWTSRLRSHINPTGTVLLQLENTMQMSLKDLL (SEQ ID NO: |
| | 56) |
| 006 | MQLRNPELHLGCALALRFLALVSWDIPGARALDNGLARTPTMGWLHWERFMCNLD |
| | CQEEPDSCISEKLFEEMAELMVSEGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWKSIKSILDWTSFNQERIVDVAGPGGWNDPDMLVIGNFGL |
| | SWNQQVTQMALWAIMAAPLFMSNDLRHISPQAKALLQDKDVIAINQDPLGKQGYQ |
| | LRQGDNFEVWERPLSGDAWAVAMINRQEIGGPRSYTIPVASLGKGVACNPACFIT |
| | QLLPVKRKLGFYEWTSRLRSHINPTGTVLLQLENTMQMSLKDLL (SEQ ID NO: |
| | 57) |
| 007 | MQLRNPELHLGCALALRFLALVSWDIPGARALDNGLARTPTMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWKSIKSILDWTSRNQERIVDVAGPGGWNDPDMLVIGNEGL |
| | SWDQQVTQMALWAIMAAPLFMSNDLRHISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRKLGFYEATSRLKSHINPTGTVLLQLENTMQMSLKDLL (SEQ ID NO: |
| | 58) |
| 008 | MQLRNPELHLGCALALRFLALVSWDIPGARALDNGLARTPTMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWASIKSILDWTSRNQERIVDVAGPGGWNDPDMLVIGNEGL |
| | SWDQQVTQMALWAIMAAPLFMSNDLRHISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRQLGFYNWTSRLKSHINPTGTVLLQLENTMQMSLKDLL (SEQ ID NO: |
| | 59) |
| 009 | MQLRNPELHLGCALALRFLALVSWDIPGARALDNGLARTPPMGWLHWERFMCNLD |
| | CQEEPDSCISEKLFEEMAERMVTDGWKDAGYEYLCIDDCWMAPQRDSEGRLQADP |
| | QRFPHGIRQLANHVHSKGLKLGIYADVGNKTCAGFPGSFGYYDIDAQTFADWGVD |
| | LLKFDGCYCDSLENLADGYKHMSLALNKTGRPIVYSCEWPLYMWPFQKPNYTEIR |
| | QYCNHWRNFADIDDSWASIKSILDWTSRNQERIVDVAGPGGWNDPDMLVIGNEGL |
| | SWDQQVTQMALWAIMAAPLFMSNDLRHISPQAKALLQDKDVIAINQDPLGKQGYQ |
| | LRKGDNFEVWERPLSGDAWAVAIINRQEIGGPRSYTIPVASLGKGVACNPACFIT |
| | QLLPVKRQLGFYNWTSRLKSHINPTGTVLLQLENTMQMSLKDLL (SEQ ID NO: |
| | 60) |
| WT with | MQLRNPELHLGCALALRFLALVSWDIPGARALDNGLARTPTMGWLHWERFMCNLD |
| signal | CQEEPDSCISEKLFMEMAELMVSEGWKDAGYEYLCIDDCWMAPQRDSEGRLQADP |
| peptide | QRFPHGIRQLANYVHSKGLKLGIYADVGNKTCAGFPGSFGYYDIDAQTFADWGVD |
| | LLKFDGCYCDSLENLADGYKHMSLALNRTGRSIVYSCEWPLYMWPFQKPNYTEIR |
| | QYCNHWRNFADIDDSWKSIKSILDWTSFNQERIVDVAGPGGWNDPDMLVIGNFGL |
| | SWNQQVTQMALWAIMAAPLFMSNDLRHISPQAKALLQDKDVIAINQDPLGKQGYQ |
| | LRQGDNFEVWERPLSGLAWAVAMINRQEIGGPRSYTIAVASLGKGVACNPACFIT |
| | QLLPVKRKLGFYEWTSRLRSHINPTGTVLLQLENTMQMSLKDLL (SEQ ID NO: |
| | 75) |

Nucleic Acid Sequences

| | |
|---|---|
| Nucleic | ctggataatggattggctagaacacctactatggggtggcttcactgggagaggt |
| acid | tcatgtgcaacctcgactgtcaggaagaaccagacagctgcatctccgagaagct |
| encoding | gtttatggaaatggccgagcgaatggtgtcagaaggctggaaagatgcaggttac |
| A | gagtatctgtgtattgacgattgctggatggctccgcaacgggacagtgagggca |
| | gacttcaggcagatcctcagcgcttcccacatgggataaggcagctcgccaacta |
| | cgtccactctaagggactgaaactgggcatctatgctgacgtggggaataagacc |
| | tgtgcgggatttcccggtagcttcggctactacgacattgatgcccagacctttg |
| | ccgattggggagttgacctcctcaaattcgatggctgctattgtgactctttgga |
| | gaacctggcagacgggtacaagcatatgtccctggccctgaatcggacaggtaga |
| | cccatcgtgtatagttgcgaatggcccctttacatgtggccttttcaaaagccaa |
| | actacactgagattcgccagtattgcaatcactggaggaacttcgctgatatcga |
| | tgactcatgggcgagcatcaaatccatattggattggacctctcggaatcaggag |
| | cgcattgtagacgtcgcaggacccggcggctggaacgaccctgatatgctggtga |
| | tcgggaattttggtcttagctgggaccagcaagttacgcagatggctctgtgggc |
| | aattatggcagccccactcttcatgtccaacgatctgcgacacatctctcctcaa |
| | gctaaggctctgctgcaggacaaagatgtgattgccatcaatcaggacccactcg |
| | gaaagcagggctatcagctgagaaaaggcgacaacttcgaagtctgggaaaggcc |
| | actttcaggagacgcatgggctgtggccataataaaccggcaagagattggtggg |
| | cccaggagctacacaatccccgttgccagtttgggcaagggagtggcgtgtaatc |
| | ctgcctgctttatcactcagctgctcccagtcaaaagacagctggggttctatga |
| | gtggacctcccgcctcaagagccatattaatcccacaggtaccgtactgctgcaa |
| | cttgaaaacacgatgcagatgagtttgaaggacctcctgtag (SEQ ID NO: 18) |
| Nucleic | ctggataatggattggctagaacacctcctatggggtggcttcactgggagaggt |
| acid | tcatgtgcaacctggactgtcaggaagaaccagacagctgcatctccgagaagct |
| encoding | ctttgaggaaatggccgaacgaatggtgactgagggctggaaagatgcaggttac |
| B | gagtatctgtgtattgacgattgctggatggccccacaacgggattctgagggaa |
| | gacttcaggctgatccgcagcgcttccctcatggcataaggcagctggcaaacca |
| | cgtccacagtaaggggctcaaattgggaatctacgcggacgtgggcaataagacc |
| | tgtgccggttttccgggatcattcgggtattatgacattgacgcccaaacgtttg |
| | ctgattggggcgttgacctgctgaaattcgatggttgctactgtgacagcctcga |
| | aaacctggcagacggctacaagcatatgtctctcgccctgaatagaaccggtcgg |
| | ccaatcgtatattcctgcgagtggcctctttacatgtggccatttcagaaaccga |
| | actacacagaaattcgccagtattgcaatcattggaggaacttcgctgatatcga |
| | tgactcatgggcctccataaagagcatcttggactggaccagtcggaatcaggag |
| | cgaattgtggatgtcgcaggccctggaggatggaacgatccagacatgctggtga |
| | tcggcaattttggcctctcttgggaccagcaggttacccaaatggctctgtgggc |
| | aattatggccggtcctcttttcatgagcaacgatctgcgcgcgatctcaccacag |
| | gcaaaggccctgctccaagacaaagatgtgatagccatcaatcaggacccgttgg |
| | gaaagcagggctaccagctgagaaaaggcgacaactttgaggtctgggaaaggcc |
| | tttgagtggagatgcgtgggctgtggccattattaatcggcaagagatcggaggt |
| | ccacgctcctatacaattcctgtagcatctcttggcaagggcgttgcctgtaacc |
| | cagcttgcttcatcactcagcttctgccggtgaagagaaaactgggtttctacga |
| | agctaccagcaggctccgatcccacatcaaccctacaggaaccgtcctcttgcag |
| | ctggagaatacgatgcagacttcactgaaggacctcctgtag (SEQ ID NO: 19) |
| Nucleic | ctggataatggattggctagaacacctcctatggggtggcttcactgggagaggt |
| acid | tcatgtgcaacctggactgtcaggaagaaccagacagctgcatctccgagaagct |
| encoding | ctttgaggaaatggccgaacgaatggtgactgagggctggaaagatgcaggttac |
| C | gagtatctgtgtattgacgattgctggatggccccacaacgggattctgagggaa |
| | gacttcaggctgatccgcagcgcttccctcatggcataaggcagctggcaaacca |
| | cgtccacagtaaggggctcaaattgggaatctacgcggacgtgggcaataagacc |
| | tgtgccggttttccgggatcattcgggtattatgacattgacgcccaaacgtttg |
| | ctgattggggcgttgacctgctgaaattcgatggttgctactgtgacagcctcga |
| | aaacctggcagacggctacaagcatatgtctctcgccctgaataaaaccggtcgg |
| | ccaatcgtatattcctgcgagtggcctctttacatgtggccatttcagaaaccga |
| | actacacagaaattcgccagtattgcaatcattggaggaacttcgctgatatcga |
| | tgactcatgggcctccataaagagcatcttggactggaccagtcggaatcaggag |
| | cgaattgtggatgtcgcaggccctggaggatggaacgatccagacatgctggtga |
| | tcggcaattttggcctctcttgggaccagcaggttacccaaatggctctgtgggc |
| | aattatggccggtcctcttttcatgagcaacgatctgcgcgcgatctcaccacag |
| | gcaaaggccctgctccaagacaaagatgtgatagccatcaatcaggacccgttgg |
| | gaaagcagggctaccagctgagaaaaggcgacaactttgaggtctgggaaaggcc |
| | tttgagtggagatgcgtgggctgtggccattattaatcggcaagagatcggaggt |
| | ccacgctcctatacaattcctgtagcatctcttggcaagggcgttgcctgtaacc |
| | cagcttgcttcatcactcagcttctgccggtgaagagaaaactgggtttctacga |
| | agctaccagcaggctccgatcccacatcaaccctacaggaaccgtcctcttgcag |
| | ctggagaatacgatgcagacttcactgaaggacctcctgtag (SEQ ID NO: 20) |
| Nucleic | ctggataatggattggctagaacacctcctatggggtggcttcactgggagaggt |
| acid | tcatgtgcaacctggactgtcaggaagaaccagacagctgcatctccgagaagct |
| encoding | ctttgaggaaatggccgaacgaatggtgactgacggctggaaagatgcaggttac |
| D | gagtatctgtgtattgacgattgctggatggccccacaacgggattctgagggaa |
| | gacttcaggctgatccgcagcgcttccctcatggcataaggcagctggcaaacca |
| | cgtccacagtaaggggctcaaattgggaatctacgcggacgtgggcaataagacc |
| | tgtgccggttttccgggatcattcgggtattatgacattgacgcccaaacgtttg |
| | ctgattggggcgttgacctgctgaaattcgatggttgctactgtgacagcctcga |
| | aaacctggcagacggctacaagcatatgtctctcgccctgaataaaaccggtcgg |
| | ccaatcgtatattcctgcgagtggcctctttacatgtggccatttcagaaaccga |
| | actacacagaaattcgccagtattgcaatcattggaggaacttcgctgatatcga |
| | tgactcatgggcctccataaagagcatcttggactggaccagtcggaatcaggag |
| | cgaattgtggatgtcgcaggccctggaggatggaacgatccagacatgctggtga |
| | tcggcaattttggcctctcttgggaccagcaggttacccaaatggctctgtgggc |
| | aattatggccggtcctcttttcatgagcaacgatctgcgcgcgatctcaccacag |
| | gcaaaggccctgctccaagacaaagatgtgatagccatcaatcaggacccgttgg |
| | gaaagcagggctaccagctgagaaaaggcgacaactttgaggtctgggaaaggcc |
| | tttgagtggagatgcgtgggctgtggccattattaatcggcaagagatcggaggt |
| | ccacgcggttatacaattcctgtagcatctcttggcaagggcgttgcctgtaacc |
| | cagcttgcttcatcactcagcttctgccggtgaagagaaaactgggtttctacga |
| | agctaccagcaggctccgatcccacatcaaccctacaggaaccgtcctcttgcag |
| | ctggagaatacgatgcagacttcactgaaggacctcctgtag (SEQ ID NO: 21) |
| Codon- | atgcagctgaggaacccagaactacatctgggctgcgcgcttgcgcttcgcttcc |
| optimized | tggccctcgtttcctgggacatccctggggctagagcactggataatggactggc |
| nucleic | cagaactcctcccatgggctggctgcactgggaaaggtttatgtgtaatctggac |
| acid | tgtcaagaggagccagattcctgcatctctgagaagctctttgaagagatggctg |
| encoding | agaggatggtgacagatggttggaaggatgctggttatgagtacctgtgcattga |
| D | tgactgctggatggccccccagagagattcagaaggcagactgcaagcagaccct |
| (D1) | cagaggttcccccatggcatccgccagcttgccaaccatgtccactccaagggcc |
| | tgaaactgggtatctatgctgatgtgggcaataagacctgtgctggctttcctgg |
| | ctcctttggctactatgacattgacgcccagacctttgctgactggggtgtggac |
| | ttgctgaagtttgatggctgctactgtgactccctggagaacctggcagatggat |
| | acaagcacatgtctctggctctgaacaagactggcagacccattgtttacagctg |
| | tgagtggccactgtacatgtggcccttccagaagcccaactacactgagatcaga |
| | cagtactgcaatcattggaggaactttgccgacatcgatgattcttgggcctcca |
| | tcaagagcatcctggactggacatccagaaaccaagaaagaattgtggatgtggc |
| | tggacctggaggatggaatgatcctgacatgctggtgattggaaattttgggctg |
| | tcctgggaccagcaagtgactcagatggccctctgggccatcatggctgggcccc |
| | tcttcatgagcaatgacctgagggccatttccccccaagccaaggccctgcttca |
| | agacaaagatgtcattgctatcaatcaagatcccctggggaagcaaggctaccag |
| | ctcagaaaaggagacaacttcgaggtgtgggagagacctctgtctggagatgcct |
| | gggctgtggccatcatcaacagacaagagattggtggccccagaggttacaccat |
| | ccctgttgcttctcttggcaagggtgttgcctgcaacccagcttgcttcatcacc |
| | cagctgctcccagtgaagaggaagctgggcttctatgaagctacctctaggttga |
| | gatcccacatcaaccccactggtactgtgctgctgcagctggaaaacaccatgca |
| | gacttccctcaaggacctcctgtga (SEQ ID NO: 35) |
| Codon- | atgcagctgaggaacccagaactacatctgggctgcgcgcttgcgcttcgcttcc |
| optimized | tggccctcgtttcctgggacatccctggggctagagcactggataatggacttgc |
| nucleic | aagaactcctcccatgggctggctgcactgggagaggtttatgtgtaatctggac |
| acid | tgtcaagaagaaccagattcctgcatctctgagaagctctttgaggagatggctg |
| encoding | agaggatggtgacagatggatggaaggatgctggttatgaatatctgtgcattga |
| D | tgactgctggatggcccctcagagagacagtgaaggccgcctgcaagcagacccc |
| | cagaggttcccccatggaatcagacagttggccaaccatgtccactccaagggcc |
| (D2) | tgaaactgggcatctatgctgatgtgggcaataagacctgtgctggcttccctgg |
| | ctcctttgggtactatgacattgacgcccagacttttgctgactggggtgtggac |
| | ttgctgaagtttgatggctgctactgtgactccctggaaaacctggcagatggtt |
| | acaagcacatgtctctggccctgaacaagactggcagacccattgtttacagctg |
| | tgagtggccactgtacatgtggcccttccagaagcccaactacactgagatccgc |
| | cagtactgcaatcattggaggaactttgcagacatcgatgattcttgggccagca |
| | tcaagtccatcctggactggacttccagaaaccaagagagaattgtggatgttgc |
| | tggacctggaggttggaatgatcctgacatgctggtgattggaaattttggactg |
| | tcctgggaccagcaagtgactcagatggccctctgggccatcatggctgggcccc |
| | tcttcatgagcaatgacctgagggccatttccccccaagccaaggctctgcttca |
| | agacaaagatgtcattgctattaatcaagatcccctggggaagcaaggctaccag |
| | ctcagaaaaggagacaacttcgaggtgtgggaaagacctctgtctggagatgcct |
| | gggctgtggctatcatcaacagacaagaaattggtggccccagaggctacaccat |
| | ccctgtggcctctcttggcaagggtgttgcctgcaacccagcttgcttcatcacc |
| | cagctgctcccagtgaagaggaagctgggtttctatgaggctacctctaggttga |
| | gatcccacatcaatcccactggtacagtgctgctgcagctggagaacaccatgca |
| | gacctccctcaaggacctcctttaa (SEQ ID NO: 36) |
| Codon- | atgcagctgaggaacccagaactacatctgggctgcgcgcttgcgcttcgcttcc |
| optimized | tggccctcgtttcctgggacatccctggggctagagcattggacaatggactggc |
| nucleic | aagaactcctcccatgggctggctgcactgggaaaggtttatgtgtaatctggac |
| acid | tgtcaagaggagccagattcctgcatctctgagaagctctttgaagagatggctg |
| encoding | agaggatggtgacagatggatggaaggatgctggatatgagtacctgtgcattga |
| D | tgactgctggatggcccctcagagagacagtgaaggcagactgcaagcagacccc |
| (D3) | cagaggttcccccatggcattaggcagctggccaaccatgtccactccaagggcc |
| | tgaaactgggcatctatgctgatgtgggcaataagacctgtgctggtttccctgg |
| | ctcctttggatactatgacattgatgcccagacctttgctgactggggtgtggac |
| | ctgctcaagtttgatggctgctactgtgactccctggagaacctggctgatggtt |
| | acaagcacatgtctcttgctctgaacaagactggtagacccattgtttacagctg |
| | tgagtggccactgtacatgtggcccttccagaagcccaactacactgagatcaga |
| | cagtactgcaatcattggaggaattttgcagatattgatgattcttgggcctcca |
| | tcaagagcatcctggactggacttccagaaaccaagaaagaattgtggatgttgc |
| | tggccctggaggttggaatgaccctgacatgctggtgattggcaactttgggctg |
| | tcctgggaccagcaagtgactcagatggccctctgggccatcatggctgggcccc |
| | tcttcatgtccaatgatctgagggccatttccccccaagccaaggccctgcttca |
| | agacaaagatgtcattgctatcaatcaagatcccctgggcaagcaaggctaccag |
| | ctcagaaaaggagacaactttgaggtgtgggagagacctctgtctggagatgcct |
| | gggctgtggccatcatcaacagacaagagattggtggccccagaggctacaccat |
| | ccctgtggcttccctggggaagggtgttgcctgcaacccagcttgcttcatcacc |
| | cagcttctgccagtgaagaggaagctgggcttctatgaagctaccagcagactga |
| | gatcccacatcaaccccactggcacagtgctgctgcagctggaaaacaccatgca |
| | gacttctctgaaggacctcctgtga (SEQ ID NO: 37) |
| Codon- | atgcagctgaggaacccagaactacatctgggctgcgcgcttgcgcttcgcttcc |
| optimized | tggccctcgtttcctgggacatccctggggctagagcactggataatggactggc |
| nucleic | aagaactcctcccatgggctggctgcattgggagaggtttatgtgtaatctggac |
| acid | tgtcaagaggagccagactcctgcatctctgagaagctctttgaagagatggctg |
| encoding | agaggatggtgacagatggttggaaggatgctggatatgagtacctgtgcattga |
| D | tgactgctggatggccccccagagagacagtgaaggcagactgcaagcagaccct |
| (D4) | cagaggttcccccatggcattaggcagctggccaaccatgtccactccaagggcc |
| | tgaaacttggcatctatgctgatgtgggcaataagacctgtgctggcttccctgg |
| | ctcctttggctactatgacattgatgcccagacttttgctgactggggtgtggac |
| | ctgctcaagtttgatggctgctactgtgactctctggaaaacctggctgatggat |
| | acaagcacatgtctttggctctgaacaagactggtaggcccattgtttacagctg |
| | tgagtggccactgtacatgtggcccttccagaagcccaactacactgagatcaga |
| | cagtactgcaaccactggaggaactttgcagatattgatgattcctgggcctcca |
| | tcaagagcatcctggactggacttccagaaaccaagagagaattgtggatgttgc |
| | tgggcctggaggatggaatgatcctgacatgctggtgattggaaattttgggctg |
| | agctgggaccagcaagtgactcagatggccctctgggccatcatggctggtcccc |
| | tcttcatgagcaatgacttgagggccatttccccccaagccaaggccctgcttca |
| | agacaaagatgtcattgctatcaatcaagatcccctgggcaagcaaggctaccag |
| | ttgagaaaaggagacaactttgaggtgtgggaaagacctctgtctggagatgcct |
| | gggctgtggccatcatcaacagacaagaaattggtggccccagaggttacaccat |
| | ccctgtggcttctcttggaaaaggggttgcctgcaatccagcttgcttcatcacc |
| | cagctcctgccagtgaagaggaagctgggtttctatgaagctacctctaggctca |
| | gatcccacatcaaccccactggcacagtgctgctgcagctggagaacaccatgca |
| | gacctccctgaaggacctcctttaa (SEQ ID NO: 38) |
| Codon- | atgcagctgaggaacccagaactacatctgggctgcgcgcttgcgcttcgcttcc |
| optimized | tggccctcgtttcctgggacatccctggggctagagcactggataatggactggc |
| nucleic | aagaactcctcccatgggctggctgcactgggaaaggtttatgtgtaatctggac |
| acid | tgtcaagaggagcctgactcctgcatctctgagaagctctttgaagagatggctg |
| encoding | agaggatggtgacagatggatggaaggatgctggttatgagtacctgtgcattga |
| D | tgactgctggatggcccctcagagagattctgaaggcagactgcaagcagacccc |
| (D5) | cagaggttcccccatggcattaggcagctggccaaccatgtccactccaagggcc |
| | tgaaactgggcatctatgctgatgtgggcaataagacctgtgctggctttcctgg |
| | ctcctttggatactatgacattgatgcccagacctttgctgactggggtgtggac |
| | ctcctgaagtttgatggctgctactgtgactctctggagaacctggctgatggtt |
| | acaagcacatgtctcttgctctgaacaagactggtagacccattgtttacagctg |
| | tgagtggccactgtacatgtggcccttccagaagcccaactacactgagatcaga |
| | cagtactgcaatcattggaggaattttgcagatattgatgattcctgggcctcca |
| | tcaagagcatcctggactggacatccagaaaccaagaaagaattgtggatgtggc |
| | tgggcctggaggttggaatgacccagacatgctggtgattggcaactttgggctg |
| | agctgggaccagcaagtgactcagatggccctctgggccatcatggctggacctc |
| | tcttcatgtccaatgatctgagagccatttccccccaagccaaggccctgctcca |
| | agacaaagatgtcattgctatcaatcaagatcccctggggaagcaaggctaccag |
| | ctcagaaagggtgacaactttgaggtgtgggagagacctctgtctggagatgcct |
| | gggctgtggccatcatcaacagacaagagattggtggccccagaggctacaccat |
| | ccctgttgcttccctgggaaaaggagtggcctgcaacccagcttgcttcatcacc |
| | cagcttctgcctgtgaagaggaagctgggcttctatgaagctacctctaggctga |
| | ggtcccacatcaaccccactggcacagtgctgctgcagctggaaaacaccatgca |
| | gacttccctcaaggacctgctttaa (SEQ ID NO: 39) |
| Codon- | atgcagctgaggaacccagaactacatctgggctgcgcgcttgcgcttcgcttcc |
| optimized | tggccctcgtttcctgggacatccctggggctagagcactggacaatggcctggc |
| nucleic | cagaacccctcccatgggctggctgcactgggagagattcatgtgcaacctggat |
| acid | tgccaggaggagccagactcttgcatctctgaaaagctgtttgaggaaatggccg |
| encoding | agagaatggtgacagatggatggaaggatgccggatacgagtacctgtgtatcga |
| D | tgactgttggatggccccccagagagactccgagggccgtctgcaggctgaccca |
| (D6) | cagaggtttcctcatggaattaggcagttggccaaccatgtgcactccaagggac |
| | tgaagctgggcatctatgccgatgtgggcaacaagacctgtgctggcttcccagg |
| | cagctttggctattatgatattgatgcacaaacttttgcagactggggagttgat |
| | ctgctgaaatttgatgggtgttactgtgactccctggagaacctcgccgacggat |
| | acaagcatatgtcccttgctctgaacaagactggcaggcccattgtctactcttg |
| | tgagtggccactgtacatgtggcccttccagaagcccaactataccgagattcgc |
| | cagtactgcaatcactggaggaactttgcagacattgatgacagctgggcctcca |
| | ttaagtctatcctggattggacaagcagaaaccaagagagaattgtggatgtggc |
| | tggccctggtggttggaatgaccccgatatgctggtgattggcaactttggactg |
| | tcttgggaccagcaggtgacccagatggctctgtgggctatcatggccggccccc |
| | tcttcatgtctaatgacctgagggccatctctcctcaggccaaggcacttctgca |
| | ggataaggacgtgattgccatcaatcaggatcctctggggaagcagggctatcag |
| | ctgagaaagggggacaatttcgaggtctgggagagacccctgagcggcgatgctt |
| | gggctgtggccatcatcaatagacaagagattggagggccaagaggctacaccat |
| | tcctgtggcaagcctaggaaaaggggttgcttgcaatccagcctgtttcatcacc |
| | cagctgctgcctgttaagagaaagttgggcttctatgaggccacctctaggctga |
| | ggtcccatatcaaccccactggcacagtgctgctgcagcttgaaaacaccatgca |
| | gACCtccctcaaggacctgctgtaa (SEQ ID NO: 40) |
| nucleic | atgcagctgaggaacccagaactacatctgggctgcgcgcttgcgcttcgcttcc |
| acid | tggccctcgtttcctgggacatccctggggctagagcactggataatggattggc |
| encoding | tagaacacctcctatggggtggcttcactgggagaggttcatgtgcaacctggac |
| E | tgtcaggaagaaccagacagctgcatctccgagaagctctttgaggaaatggccg |
| | aacgaatggtgactgacggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaataaaaccggtcggccaatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggtcctc |
| | ttttcatgagcaacgatctgcgcgcgatctcaccacaggcaaaggccctgctcca |
| | agacaaagatgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgcggttatacaat |
| | tcctgtagcaaagcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 22) |
| nucleic | atgcagctgaggaacccagaactacatctgggctgcgcgcttgcgcttcgcttcc |
| acid | tggccctcgtttcctgggacatccctggggctagagcactggataatggattggc |
| encoding | tagaacacctcctatggggtggcttcactgggagaggttcatgtgcaacctggac |
| F | tgtcaggaagaaccagacagctgcatctccgagaagctctttgaggaaatggccg |
| | aacgaatggtgactgacggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaataaaaccggtcgggatatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggccctc |
| | ttttcatgagcaacgatctgcgcgcgatctcaccacaggcaaaggccctgctcca |
| | agacacggacgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgcggttatacaat |
| | tcctgtagcaaagcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 23) |
| nucleic | ttggacaatgggctggccaggacacctactatgggctggctccactgggagcgct |
| acid | ttatgtgtaacctcgactgccaagaggagccagactcatgcatctctgagaagtt |
| encoding | gttcatggagatggctgagctgatggtgagcgaagggtggaaggatgcgggctat |
| 002 | gagtatctctgtattgatgactgctggatggctccacagcgcgacagtgaaggcc |
| | ggctccaggccgatcctcagcggttcccccacggtatcagacaactggcgaatta |
| | cgtgcactcaaaaggccttaagctgggtatatatgctgatgtgggtaataaaaca |
| | tgtgcaggcttcccaggctcttttgggtactatgacatcgacgcccagacttttg |
| | cggactggggcgtggacctgctcaagtttgacggatgttactgtgactcccttga |
| | gaacctggccgacgggtacaagcatatgtcactggccctgaatcggacaggccga |
| | tccatcgtatactcttgcgagtggcctctgtacatgtggcccttccagaagccca |
| | actatacagaaatcaggcaatactgcaaccattggcggaacttcgcagacataga |
| | cgacagctgggctagcattaagtctattctggattggaccagtttcaatcaagaa |
| | aggattgtcgatgtcgcaggcccaggaggttggaatgacccagacatgctcgtga |
| | ttggaaatttcggtctgtcatgggaccaacaggtgactcagatggctctgtgggc |
| | aatcatggcagcaccactgttcatgagcaatgatttgcgacacatctcccctcag |
| | gcgaaagcccttctgcaggataaggacgttatcgccattaaccaggacccgctcg |
| | gtaagcaagggtaccagttgcgccagggagacaatttcgaggtctgggaacgacc |
| | cctgtctggactcgcttgggccgtagccgtaattaaccgacaagaaatcggcgga |
| | ccgcggagctataccatagctgtcgcctccctcggcgggggcgtagcttgtaacc |
| | cggcttgtttcataacccagctgctgcccgtcaagaggaaactggggctttatga |
| | gtggacaagtcggttgaagtctcatattaacccgacagggactgttctcctccag |
| | ctggaaaacacaatgcagatgagcctgaaggatctcttgtga (SEQ ID NO: 24) |
| nucleic | tttggacaatgggctggccaggacacctactatgggctggctccactgggagcgc |
| acid | tttatgtgtaacctcgactgccaagaggagccagactcatgcatctctgagaagt |
| encoding | tgttcatggaaatggccgagcttatggtctcagaggggtggaaagacgccggata |
| 003 | tgagtatctgtgcatcgacgattgctggatggccccccaacgcgattccgaaggc |
| | cgcttgcaggctgatccgcagcgctttcctcacgggatccgccaattggcaaatt |
| | acgtgcactcaaaggggctcaagttgggcatctacgcagacgtgggaaacaagac |
| | atgtgctggatttcccgggagtttcggttattatgacattgacgcacagaccttt |
| | gctgattggggggtcgacctcctgaagtttgacggttgttattgcgacagccttg |
| | aaaatctggccgacggttacaaacacatgtcccttgcactgaatagaactggtcg |
| | gtccatcgtctatagttgtgagtggccgctttacatgtggccttttcagaaaccc |
| | aactacaccgagattcggcaatattgcaatcactggcgaaatttcgcagatatcg |
| | atgattcttgggctagtattaaatccatcctggattggacatcattcaaccagga |
| | gcgcatcgtggacgttgctggacctggcgggtggaatgatccagacatgcttgtg |
| | atcggaaacttcggtctctcttggaaccagcaagtcactcaaatggcactctggg |
| | caattatggccgcccccctctttatgtccaacgatctgaggcatatcagtcctca |
| | ggctaaagccctgctgcaagacaaggatgtgattgctatcaaccaagatcccctt |
| | ggtaaacaggggtaccagttgcgcaaaggcgacaattttgaggtgtgggaaaggc |
| | cactttcaggcgatgcatgggccgttgcaatgatcaacaggcaagaaattggcgg |
| | acccaggagctatacaataccagtggcgtcactgggtaagggagtcgcctgtaac |
| | cccgcatgcttcattactcaacttctgccagtgaaacgaaaactcggattctatg |
| | agtggacctcaagactgaggtctcatattaacccgacagggactgttctcctcca |
| | gctggaaaacacaatgcagatgagcctgaaggatctcttgtga (SEQ ID NO: |
| | 25) |
| nucleic | ttggacaatgggctggccaggacacctactatgggctggctccactgggagcgct |
| acid | ttatgtgtaacctcgactgccaagaggagccagactcatgcatctctgagaagtt |
| encoding | gttcatggagatggctgaacgcatggtttctgagggatggaaagatgcaggctac |
| 004 | gagtacctgtgtatagacgattgttggatggccccacagcgggattcagaaggta |
| | gactccaggcggatccccagagattcccacatggaatcagacagctggccaatta |
| | cgtccattccaaaggccttaagttgggtatttacgccgacgtaggcaacaagact |
| | tgtgccggatttcccggcagtttcggatactatgacattgatgcacagactttcg |
| | ctgactggggggtggacttgctcaaatttgatggctgttattgcgatagcctcga |
| | aaatctggctgatggctacaagcacatgtcactcgctctcaaccgcactgggcgc |
| | tctatagtttactcctgcgagtggcctctgtatatgtggccgttccagaaaccca |
| | attacacagaaataaggcagtattgcaatcactggcgcaactttgctgatattga |
| | tgattcctgggcctccataaagagtatcttggactggactagtcgcaatcaggaa |
| | agaattgtcgacgtcgccggaccaggcggatggaatgatcctgatatgctcgtga |
| | tcgggaacttcggactctcatgggaccagcaggtgacccagatggctagttgggc |
| | tatcatggccgcccctctgtttatgagtaacgacctccgccacatcagcccccag |
| | gccaaggcgcttctgcaggataaagatgtcatcgccatcaaccaagatcccctgg |
| | gcaaacaaggctatcagctgcggaagggagacaattttgaggtgtgggaacgccc |
| | tttgagcggagacgcctgggctgtggctattataaatcgccaggagattgggggc |
| | ccgagaagttatactatccccgttgcaagcctgggaaagggcgtcgcctgcaacc |
| | ctgcctgcttcatcacacagctgcttcctgtcaaacgccaattggggttctacaa |
| | ttggacatccagactcaaatctcatattaacccgacagggactgttctcctccag |
| | ctggaaaacacaatgcagatgagcctgaaggatctcttgtga (SEQ ID NO: 26) |
| Codon- | atgcagctgaggaacccagaactacatctgggctgcgcgcttgcgcttcgcttcc |
| optimized | tggccctcgtttcctgggacatccctggggctagagcactggataatggactggc |
| nucleic | aagaaccccaaccatgggctggctgcactgggaaagattcatgtgtaatctggac |
| acid | tgtcaagaggagcccgactcctgcatctctgagaagctcttcatggagatggctg |
| encoding | agaggatggtgagtgaaggatggaaggatgctggttatgagtacctgtgcattga |
| 004 | tgactgctggatggccccccagagagattcagagggcagactgcaagcagatcct |
| (004-1) | cagaggttcccccatggcatcagacagctggccaactatgtccactccaagggcc |
| | tgaagctgggtatctatgctgatgtgggcaacaagacctgtgctggctttcctgg |
| | ctcctttggttactatgacattgacgcccagacctttgctgactggggagtggac |
| | ctgttgaagtttgacggctgctactgtgactctctggagaacctggctgatggct |
| | acaagcacatgtctcttgccctgaacagaactggtaggagcattgtttacagctg |
| | tgagtggccactgtacatgtggcccttccagaagcccaactacactgagatccgc |
| | cagtactgcaaccattggaggaactttgcagacatcgatgattcctgggcctcca |
| | tcaagagcatcctggactggacttccagaaaccaagagagaattgtggatgttgc |
| | tggacctggaggttggaatgaccctgacatgctggtgattggaaattttgggctg |
| | tcctgggaccagcaagtgactcagatggccagctgggccatcatggcagcccctc |
| | tctttatgtccaatgatctcagacacatttccccccaagccaaggctctgctcca |
| | agacaaagatgtcattgctattaatcaagatcccctggggaagcaaggctaccag |
| | ctgaggaaaggagacaactttgaggtgtgggagagacctctgtctggagatgcct |
| | gggctgtggctatcatcaatagacaagaaattggtggccccagatcctacaccat |
| | ccctgtggcttccctgggcaagggtgtggcctgcaatccagcttgcttcatcacc |
| | cagctcctcccagtgaagaggcagcttggcttctacaactggacatctaggctca |
| | agtcccacatcaaccccactggcacagtgctgctgcagctggaaaacaccatgca |
| | gatgagcctgaaagacctcctgtga (SEQ ID NO: 41) |
| Codon- | atgcagctgaggaacccagaactacatctgggctgcgcgcttgcgcttcgcttcc |
| optimized | tggccctcgtttcctgggacatccctggggctagagcattggacaatggactggc |
| nucleic | taggaccccaaccatgggttggctgcactgggagaggtttatgtgtaatctggac |
| acid | tgtcaagaagaacctgactcctgcatctctgagaagctgttcatggagatggctg |
| encoding | agaggatggtgagtgaaggctggaaggatgctggttatgagtacctgtgcattga |
| 004 | tgactgctggatggcccctcagagagattcagagggcagacttcaagcagacccc |
| (004-2) | cagaggttcccccatggcatccgccagctggccaactatgtccactccaagggcc |
| | tgaaactgggtatctatgctgatgtgggcaacaagacctgtgctggctttcctgg |
| | ctcctttggctactatgacattgacgcccagacctttgctgactggggtgtggac |
| | ctcctcaagtttgatggctgctactgtgactctctggaaaacctggcagatggtt |
| | acaagcacatgtctcttgccctgaacagaactggtaggagcattgtttacagctg |
| | tgagtggccactgtacatgtggcccttccagaagcccaactacactgagatcaga |
| | cagtactgcaaccattggaggaattttgccgacatcgatgattcctgggccagca |
| | tcaagtccatcctggactggacttccagaaaccaagagagaattgtggatgttgc |
| | tggacctggaggatggaatgacccagacatgctggtgattggaaatttcgggctg |
| | tcctgggaccagcaagtgactcagatggcctcttgggccatcatggcagcccccc |
| | tcttcatgtccaatgatcttagacacatttccccacaagccaaggccctcctgca |
| | agacaaagatgtcattgctattaatcaagatcccctggggaagcaaggctaccag |
| | ctcagaaaaggagacaactttgaggtgtgggaaagacctctgtctggagatgcct |
| | gggctgtggctatcatcaatagacaagaaattggtggccccagatcctacaccat |
| | ccctgtggcctccctgggcaagggtgttgcctgcaatccagcttgcttcatcacc |
| | cagctgctcccagtgaagaggcagttgggcttctacaactggacatctaggttga |
| | agagccacatcaaccccactggcacagtgctgctgcagctggagaacaccatgca |
| | gatgagcctgaaggacctgctttaa (SEQ ID NO: 42) |
| Codon- | atgcagctgaggaacccagaactacatctgggctgcgcgcttgcgcttcgcttcc |
| optimized | tggccctcgtttcctgggacatccctggggctagagcactggataatggactggc |
| nucleic | aagaaccccaaccatgggttggctgcactgggagaggtttatgtgtaatctggac |
| acid | tgtcaagaggagccagactcctgcatctctgagaagctcttcatggagatggctg |
| encoding | agaggatggtgagtgaaggctggaaggatgctggttatgagtacctgtgcattga |
| 004 | tgactgctggatggcccctcagagagattcagagggcagactgcaagcagatccc |
| (004-3) | cagaggttcccccatggcattaggcaactggccaactatgtccactccaagggcc |
| | tgaagctgggcatctatgctgatgtgggcaacaagacctgtgctggcttccctgg |
| | ctcctttggctactatgatattgatgcccagacctttgctgactggggtgtggac |
| | ctgctcaagtttgatggctgctactgtgacagcctggagaacctggctgatggtt |
| | acaagcacatgtctcttgctctgaacagaactggtagaagcattgtttacagctg |
| | tgagtggccactgtacatgtggcccttccagaagcccaactacactgagatcaga |
| | cagtactgcaaccattggaggaactttgcagacattgatgattcctgggcctcca |
| | tcaagagcatcctggactggacttccagaaaccaagagagaattgtggatgttgc |
| | tggacctggaggatggaatgaccctgacatgctggtgattggaaattttgggctg |
| | tcctgggaccagcaagtgactcagatggccagctgggccatcatggcagcccccc |
| | tgttcatgagcaatgatctcagacacatttccccccaagccaaggccctgcttca |
| | agacaaagatgtcattgctattaatcaagatcctttgggcaagcaaggctaccag |
| | ctgaggaagggagacaactttgaggtgtgggaaagacctctgtctggagatgcct |
| | gggctgtggctatcatcaatagacaagaaattggtggccccagatcctacaccat |
| | ccctgttgcctctctgggaaaaggagtggcctgcaatccagcttgcttcatcacc |
| | cagctcctcccagtgaagaggcagctgggtttctacaactggacaagcagattga |
| | agtcccacatcaaccccactggcacagtgctgctgcagctggaaaacaccatgca |
| | gatgtccctgaaggacctcctgtga (SEQ ID NO: 43) |
| Codon- | atgcagctgaggaacccagaactacatctgggctgcgcgcttgcgcttcgcttcc |
| optimized | tggccctcgtttcctgggacatccctggggctagagcacttgataatggactggc |
| nucleic | aagaaccccaaccatgggctggctgcactgggaaagattcatgtgtaatctggac |
| acid | tgtcaagaagaaccagattcctgcatctctgagaagctgtttatggagatggctg |
| encoding | agaggatggtgtcagaaggatggaaggatgctggttatgagtacctgtgcattga |
| 004 | tgactgctggatggccccccagagagacagtgaaggcagacttcaagcagaccct |
| (004-4) | cagaggttcccccatggcattaggcaactagccaactatgtccactccaagggcc |
| | tgaaactgggcatctatgctgatgtgggcaacaagacctgtgctggcttccctgg |
| | ctcctttggctactatgacattgatgcccagacctttgctgactggggtgtggac |
| | ctcctcaagtttgatggctgctactgtgactccctggaaaacctggctgatggtt |
| | acaagcacatgagcctggccctgaacagaactggtaggagcattgtttacagctg |
| | tgagtggccactgtacatgtggcccttccagaagcccaactacactgagatcaga |
| | cagtactgcaaccattggaggaactttgcagatattgatgattcttgggccagca |
| | tcaagtccatcctggactggacatccagaaaccaagagagaattgtggatgttgc |
| | tggacctggaggttggaatgatcctgacatgctggtgattggaaattttgggctg |
| | tcctgggaccagcaagtgactcagatggcctcctgggccatcatggcagcccctc |
| | tcttcatgtccaatgacttgagacacatttccccccaagccaaggccctcctgca |
| | agacaaagatgtcattgctattaatcaagatcccttgggcaagcaaggctaccag |
| | ctgaggaagggagacaactttgaggtgtgggagaggcccctgtctggagatgcct |
| | gggctgtggctatcatcaatagacaagaaattggtggccccagatcctacaccat |
| | ccctgttgcctctcttggcaaaggagtggcctgcaatccagcttgcttcatcacc |
| | cagctccttcctgtgaagaggcagctgggtttctacaactggacttcaaggttga |
| | agagccacatcaaccccactggcacagtgctgctgcagctggagaacaccatgca |
| | gatgtctctgaaggacctgctttaa (SEQ ID NO: 44) |
| Codon- | atgcagctgaggaacccagaactacatctgggctgcgcgcttgcgcttcgcttcc |
| optimized | tggccctcgtttcctgggacatccctggggctagagcactggataatggactggc |
| nucleic | aagaaccccaaccatgggctggctgcactgggagaggttcatgtgtaatctggac |
| acid | tgtcaagaggagcctgactcctgcatctctgagaagctgtttatggagatggctg |
| encoding | agaggatggtgtctgaaggatggaaggatgctggctatgagtacctgtgcattga |
| 004 | tgactgctggatggcccctcagagggacagtgaaggcagactgcaagcagacccc |
| (004-5) | cagagattcccccatggcattagacagcttgccaactatgtccactccaagggcc |
| | tgaaactgggcatctatgctgatgtgggcaacaagacctgtgctggtttccctgg |
| | ctcctttggctactatgacattgatgcccagacctttgctgactggggtgtggac |
| | ctcctcaagtttgatggctgctactgtgacagcctggaaaacctggctgatggtt |
| | acaagcacatgtctctggccctgaacagaactggtaggagcattgtttacagctg |
| | tgagtggccactgtacatgtggcccttccagaagcccaactacactgagatcaga |
| | cagtactgcaaccattggaggaactttgcagatattgatgattcctgggcctcca |
| | tcaagagcatcctggactggacttccagaaaccaagagagaattgtggatgtagc |
| | tggacctggaggttggaatgacccagacatgctggtgattggaaattttgggctg |
| | tcctgggaccagcaagtgactcagatggccagctgggccatcatggcagcccccc |
| | tcttcatgagcaatgatctcagacacatttccccccaagccaaggctctgctcca |
| | agacaaagatgtcattgctattaatcaagatcccctggggaagcaaggctaccag |
| | ctgaggaagggagacaactttgaggtgtgggaaagacctctgtctggagatgcct |
| | gggctgtggctatcatcaatagacaagaaattggtggccccagatcctacaccat |
| | ccctgttgcttctcttggcaaaggagtggcctgcaatccagcttgcttcatcacc |
| | cagctcctgcctgtgaagaggcagttgggcttctacaactggacatctaggttga |
| | agtcccacatcaaccccactggcacagtgctgctgcagctggagaacaccatgca |
| | gatgtccctgaaggacctgctttaa (SEQ ID NO: 45) |
| Nucleic | ttggacaatgggctggccaggacacctactatgggctggctccactgggagcgct |
| acid | ttatgtgtaacctcgactgccaagaggagccagactcatgcatctctgagaagtt |
| encoding | gttcgaggagatggcagaacgaatggtgacagatggatggaaggacgctggctac |
| 005 | gagtatctgtgcatagatgattgttggatggcccctcagcgagactcagagggga |
| | gactccaggccgacccccagcgatttccacacggaatccggcaactggctaacca |
| | tgtgcactcaaaagggctcaagctgggaatttatgctgacgtcgggaacaaaact |
| | tgtgcggggtttcccggctccttcggatattacgacatcgacgcccagactttcg |
| | cagactggggtgtggacctgcttaagttcgacggctgttactgcgatagtctgga |
| | aaacttggctgacggctataagcacatgagtctcgccctgaaccgaacaggcaga |
| | agcatagtctactcctgcgaatggccactttacatgtggccattccagaaaccta |
| | attataccgagatcagacaatactgtaaccattggcgaaacttcgccgacattga |
| | cgatagttggaagtcaatcaagtccatcctggattggacctctaggaaccaggaa |
| | aggatcgtggacgtggctggacctggcggatggaacgatccagacatgctcgtga |
| | taggaaactttggactgtcatggaatcagcaagtaacacagatggcgctctgggc |
| | cattatggctgcccccttgtttatgtctaacgacctgaggcatatctctcctcaa |
| | gccaaggcactcctgcaggacaaggacgttatcgccatcaaccaggacccactgg |
| | gcaagcagggataccagctgcggaaaggtgataacttcgaggtctgggagcgacc |
| | gctttcaggagacgcctgggcagttgcaatcatcaacaggcaagaaattggtggg |
| | ccacggtcttatactattcccgtggcttctctcggtaagggcgtcgcctgcaacc |
| | ccgcctgctttatcacccaattgttgcccgttaagagaaaactgggattttacga |
| | gtggacatccaggctgcggtctcatattaacccgacagggactgttctcctccag |
| | ctggaaaacacaatgcagatgagcctgaaggatctcttgtga (SEQ ID NO: 27) |
| Nucleic | ttggacaatgggctggccaggacacctactatgggctggctccactgggagcgct |
| acid | ttatgtgtaacctcgactgccaagaggagccagactcatgcatctctgagaagtt |
| encoding | gttcgaagagatggctgaactgatggtgtccgaggggtggaaggatgcagggtac |
| 006 | gagtacctttgcatagacgattgctggatggcaccacagcgggatagtgagggca |
| | ggttgcaggcggacccccaaagatttccacatggcatcagacagctggccaacca |
| | cgtgcactctaaaggcctgaagctggggatttacgccgatgtcggtaataaaaca |
| | tgtgctggtttccccggtagctttggatactacgacatcgacgcccagacatttg |
| | ctgattggggagtggacctgctcaagttcgacggctgctactgcgattctctgga |
| | aaatctggccgatgggtataagcacatgagtcttgctcttaatagaaccggacgc |
| | tctatagtctattcatgtgagtggccactgtacatgtggccatttcagaaaccca |
| | actataccgaaattagacagtattgtaaccactggcggaatttcgccgacataga |
| | cgatagctggaagagcatcaagtcaattctcgattggacttcattcaaccaggag |
| | cggatcgtcgacgtggccggccctgggggctggaatgatccagatatgctggtga |
| | tcggaaactttggtctctcctggaatcaacaggtcactcagatggccctctgggc |
| | cattatggcagcaccactctttatgagcaacgacctgagacacatttccccacag |
| | gccaaggcattgctgcaggacaaagacgtgatcgccattaaccaggacccactgg |
| | gcaagcaagggtaccaacttaggcagggagataattttgaggtctgggagcgccc |
| | actcagcggagacgcctgggccgttgccatgataaacagacaggagattggcggc |
| | cccagatcctacacaattcctgtcgctagcctgggcaagggggtggcttgtaatc |
| | ccgcctgctttataacccagctgctgccagtgaaacggaaactcgggttctatga |
| | atggacttctaggctcaggtctcatattaacccgacagggactgttctcctccag |
| | ctggaaaacacaatgcagatgagcctgaaggatctcttgtga (SEQ ID NO: 28) |
| Nucleic | ttggacaatgggctggccaggacacctactatgggctggctccactgggagcgct |
| acid | ttatgtgtaacctcgactgccaagaggagccagactcatgcatctctgagaagtt |
| encoding | gttcgaggagatggcagaacgaatggtgacagatggatggaaggacgctggctac |
| 007 | gagtatctgtgcatagatgattgttggatggcccctcagcgagactcagagggga |
| | gactccaggccgacccccagcgatttccacacggaatccggcaactggctaacca |
| | tgtgcactcaaaagggctcaagctgggaatttatgctgacgtcgggaacaaaact |
| | tgtgcggggtttcccggctccttcggatattacgacatcgacgcccagactttcg |
| | cagactggggtgtggacctgcttaagttcgacggctgttactgcgatagtctgga |
| | aaacttggctgacggctataagcacatgagtctcgccctgaaccgaacaggcaga |
| | agcatagtctactcctgcgaatggccactttacatgtggccattccagaaaccta |
| | attataccgagatcagacaatactgtaaccattggcgaaacttcgccgacattga |
| | cgatagttggaagtcaatcaagtccatcctggattggacctctaggaaccaggaa |
| | aggatcgtggacgtggctggacctggcggatggaacgatccagacatgctcgtga |
| | taggaaactttggactgtcatgggatcagcaagtaacacagatggcgctctgggc |
| | cattatggctgcccccttgtttatgtctaacgacctgaggcatatctctcctcaa |
| | gccaaggcactcctgcaggacaaggacgttatcgccatcaaccaggacccactgg |
| | gcaagcagggataccagctgcggaaaggtgataacttcgaggtctgggagcgacc |
| | gctttcaggagacgcctgggcagttgcaatcatcaacaggcaagaaattggtggg |
| | ccacggtcttatactattcccgtggcttctctcggtaagggcgtcgcctgcaacc |
| | ccgcctgctttatcacccaattgttgcccgttaagagaaaactgggattttacga |
| | ggcgacatccaggctgaagtctcatattaacccgacagggactgttctcctccag |
| | ctggaaaacacaatgcagatgagcctgaaggatctcttgtga (SEQ ID NO: 29) |
| Nucleic | tggacaatggattggcaaggacgcctaccatgggctggctgcactgggagcgctt |
| acid | catgtgcaaccttgactgccaggaagagccagattcctgcatcagtgagaagctc |
| encoding | ttcgaggagatggcagagagaatggtcaccgacggctggaaggatgcaggttatg |
| 008 | agtacctctgcattgatgactgttggatggctccccaaagagattcagaaggcag |
| | acttcaggcagaccctcagcgctttcctcatgggattcgccagctagctaatcac |
| | gttcacagcaaaggactgaagctagggatttatgcagatgttggaaataaaacct |
| | gcgcaggcttccctgggagttttggatactacgacattgatgcccagacctttgc |
| | tgactggggagtagatctgctaaaatttgatggttgttactgtgacagtttggaa |
| | aatttggcagatggttataagcacatgtccttggccctgaataggactggcagaa |
| | gcattgtgtactcctgtgagtggcctctttatatgtggccctttcaaaagcccaa |
| | ttatacagaaatccgacagtactgcaatcactggcgaaattttgctgacattgat |
| | gattcctgggccagtataaagagtatcttggactggacatctagaaaccaggaga |
| | gaattgttgatgttgctggaccagggggttggaatgacccagatatgttagtgat |
| | tggcaactttggcctcagctgggaccagcaagtaactcagatggccctctgggct |
| | atcatggctgctcctttattcatgtctaatgacctccgacacatcagccctcaag |
| | ccaaagctctccttcaggataaggacgtaattgccatcaatcaggaccccttggg |
| | caagcaagggtaccagcttagaaagggagacaactttgaagtgtgggaacgacct |
| | ctctcaggcgatgcctgggctgtagctatcataaaccggcaggagattggtggac |
| | ctcgctcttataccatccccgttgcttccctgggtaaaggagtggcctgtaatcc |
| | tgcctgcttcatcacacagctcctccctgtgaaaaggcagctagggttctataac |
| | tggacttcaaggttaaagagtcacataaatcccacaggcactgttttgcttcagc |
| | tagaaaatacaatgcagatgtcattaaaagacttactttaa (SEQ ID NO: 31) |
| Nucleic | tggacaatggattggcaaggacgcctCCCatgggctggctgcactgggagcgctt |
| acid | catgtgcaaccttgactgccaggaagagccagattcctgcatcagtgagaagctc |
| encoding | ttcgaggagatggcagagagaatggtcaccgacggctggaaggatgcaggttatg |
| 009 | agtacctctgcattgatgactgttggatggctccccaaagagattcagaaggcag |
| WT | acttcaggcagaccctcagcgctttcctcatgggattcgccagctagctaatcac |
| | gttcacagcaaaggactgaagctagggatttatgcagatgttggaaataaaacct |
| | gcgcaggcttccctgggagttttggatactacgacattgatgcccagacctttgc |
| | tgactggggagtagatctgctaaaatttgatggttgttactgtgacagtttggaa |
| | aatttggcagatggttataagcacatgtccttggccctgaataagactggcagac |
| | ccattgtgtactcctgtgagtggcctctttatatgtggccctttcaaaagcccaa |
| | ttatacagaaatccgacagtactgcaatcactggcgaaattttgctgacattgat |
| | gattcctgggccagtataaagagtatcttggactggacatctagaaaccaggaga |
| | gaattgttgatgttgctggaccagggggttggaatgacccagatatgttagtgat |
| | tggcaactttggcctcagctgggaccagcaagtaactcagatggccctctgggct |
| | atcatggctgctcctttattcatgtctaatgacctccgacacatcagccctcaag |
| | ccaaagctctccttcaggataaggacgtaattgccatcaatcaggaccccttggg |
| | caagcaagggtaccagcttagaaagggagacaactttgaagtgtgggaacgacct |
| | ctctcaggcgatgcctgggctgtagctatcataaaccggcaggagattggtggac |
| | ctcgctcttataccatccccgttgcttccctgggtaaaggagtggcctgtaatcc |
| | tgcctgcttcatcacacagctcctccctgtgaaaaggcagctagggttctataac |
| | tggacttcaaggttaaagagtcacataaatcccacaggcactgttttgcttcagc |
| | tagaaaatacaatgcagatgtcattaaaagacttactttaa (SEQ ID NO: 32) |
| | atgcagctgaggaacccagaactacatctgggctgcgcgcttgcgcttcgcttcc |
| | tggccctcgtttcctgggacatccctggggctagagcactggacaatggattggc |
| | aaggacgcctaccatgggctggctgcactgggagcgcttcatgtgcaaccttgac |
| | tgccaggaagagccagattcctgcatcagtgagaagctcttcatggagatggcag |
| | agctcatggtctcagaaggctggaaggatgcaggttatgagtacctctgcattga |
| | tgactgttggatggctccccaaagagattcagaaggcagacttcaggcagaccct |
| | cagcgctttcctcatgggattcgccagctagctaattatgttcacagcaaaggac |
| | tgaagctagggatttatgcagatgttggaaataaaacctgcgcaggcttccctgg |
| | gagttttggatactacgacattgatgcccagacctttgctgactggggagtagat |
| | ctgctaaaatttgatggttgttactgtgacagtttggaaaatttggcagatggtt |
| | ataagcacatgtccttggccctgaataggactggcagaagcattgtgtactcctg |
| | tgagtggcctctttatatgtggccctttcaaaagcccaattatacagaaatccga |
| | cagtactgcaatcactggcgaaattttgctgacattgatgattcctggaaaagta |
| | taaagagtatcttggactggacatcttttaaccaggagagaattgttgatgttgc |
| | tggaccagggggttggaatgacccagatatgttagtgattggcaactttggcctc |
| | agctggaatcagcaagtaactcagatggccctctgggctatcatggctgctcctt |
| | tattcatgtctaatgacctccgacacatcagccctcaagccaaagctctccttca |
| | ggataaggacgtaattgccatcaatcaggaccccttgggcaagcaagggtaccag |
| | cttagacagggagacaactttgaagtgtgggaacgacctctctcaggcttagcct |
| | gggctgtagctatgataaaccggcaggagattggtggacctcgctcttataccat |
| | cgcagttgcttccctgggtaaaggagtggcctgtaatcctgcctgcttcatcaca |
| | cagctcctccctgtgaaaaggaagctagggttctatgaatggacttcaaggttaa |
| | gaagtcacataaatcccacaggcactgttttgcttcagctagaaaatacaatgca |
| | gatgtcattaaaagacttactttaa (SEQ ID NO: 3) |

Nucleic Acid sequence Encoding α-GLA variants with signal peptide

| | |
|---|---|
| Nucleic | atgcagctgaggaacccagaactacatctgggctgcgcgcttgcgcttcgcttcc |
| acid | tggccctcgtttcctgggacatccctggggctagagcactggataatggattggc |
| sequence | tagaacacctactatggggggcttcactgggagaggttcatgtgcaacctcgac |
| encoding | tgtcaggaagaaccagacagctgcatctccgagaagctgtttatggaaatggccg |
| A | agcgaatggtgtcagaaggctggaaagatgcaggttacgagtatctgtgtattga |
| | cgattgctggatggctccgcaacgggacagtgagggcagacttcaggcagatcct |
| | cagcgcttcccacatgggataaggcagctcgccaactacgtccactctaagggac |
| | tgaaactgggcatctatgctgacgtggggaataagacctgtgcgggatttcccgg |
| | tagcttcggctactacgacattgatgcccagacctttgccgattggggagttgac |
| | ctcctcaaattcgatggctgctattgtgactctttggagaacctggcagacgggt |
| | acaagcatatgtccctggccctgaatcggacaggtagacccatcgtgtatagttg |
| | cgaatggcccctttacatgtggccttttcaaaagccaaactacactgagattcgc |
| | cagtattgcaatcactggaggaacttcgctgatatcgatgactcatgggcgagca |
| | tcaaatccatattggattggacctctcggaatcaggagcgcattgtagacgtcgc |
| | aggacccggcggctggaacgaccctgatatgctggtgatcgggaattttggtctt |
| | agctgggaccagcaagttacgcagatggctctgtgggcaattatggcagccccac |
| | tcttcatgtccaacgatctgcgacacatctctcctcaagctaaggctctgctgca |
| | ggacaaagatgtgattgccatcaatcaggacccactcggaaagcagggctatcag |
| | ctgagaaaaggcgacaacttcgaagtctgggaaaggccactttcaggagacgcat |
| | gggctgtggccataataaaccggcaagagattggtgggcccaggagctacacaat |
| | ccccgttgccagtttgggcaagggagtggcgtgtaatcctgcctgctttatcact |
| | cagctgctcccagtcaaaagacagctggggttctatgagtggacctcccgcctca |
| | agagccatattaatcccacaggtaccgtactgctgcaacttgaaaacacgatgca |
| | gatgagtttgaaggacctcctgtag (SEQ ID NO: 61) |
| Nucleic | atgcagctgaggaacccagaactacatctgggctgcgcgcttgcgcttcgcttcc |
| acid | tggccctcgtttcctgggacatccctggggctagagcactggataatggattggc |
| sequence | tagaacacctcctatggggtggcttcactgggagaggttcatgtgcaacctggac |
| encoding | tgtcaggaagaaccagacagctgcatctccgagaagctctttgaggaaatggccg |
| B | aacgaatggtgactgagggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaatagaaccggtcggccaatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggtcctc |
| | ttttcatgagcaacgatctgcgcgcgatctcaccacaggcaaaggccctgctcca |
| | agacaaagatgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgctcctatacaat |
| | tcctgtagcatctcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 62) |
| Nucleic | atgcagctgaggaacccagaactacatctgggctgcgcgcttgcgcttcgcttcc |
| acid | tggccctcgtttcctgggacatccctggggctagagcactggataatggattggc |
| sequence | tagaacacctcctatggggtggcttcactgggagaggttcatgtgcaacctggac |
| encoding | tgtcaggaagaaccagacagctgcatctccgagaagctctttgaggaaatggccg |
| C | aacgaatggtgactgagggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaataaaaccggtcggccaatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggtcctc |
| | ttttcatgagcaacgatctgcgcgcgatctcaccacaggcaaaggccctgctcca |
| | agacaaagatgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgctcctatacaat |
| | tcctgtagcatctcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 63) |
| Nucleic | atgcagctgaggaacccagaactacatctgggctgcgcgcttgcgcttcgcttcc |
| acid | tggccctcgtttcctgggacatccctggggctagagcactggataatggattggc |
| sequence | tagaacacctcctatggggtggcttcactgggagaggttcatgtgcaacctggac |
| encoding | tgtcaggaagaaccagacagctgcatctccgagaagctctttgaggaaatggccg |
| D | aacgaatggtgactgacggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaataaaaccggtcggccaatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggtcctc |
| | ttttcatgagcaacgatctgcgcgcgatctcaccacaggcaaaggccctgctcca |
| | agacaaagatgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgcggttatacaat |
| | tcctgtagcatctcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 64) |
| Nucleic | atgcagctgaggaacccagaactacatctgggctgcgcgcttgcgcttcgcttcc |
| acid | tggccctcgtttcctgggacatccctggggctagagcactggataatggattggc |
| sequence | tagaacacctcctatggggtggcttcactgggagaggttcatgtgcaacctggac |
| encoding | tgtcaggaagaaccagacagctgcatctccgagaagctctttgaggaaatggccg |
| E | aacgaatggtgactgacggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaataaaaccggtcggccaatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggtcctc |
| | ttttcatgagcaacgatctgcgcgcgatctcaccacaggcaaaggccctgctcca |
| | agacaaagatgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgcggttatacaat |
| | tcctgtagcaaagcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 65) |
| Nucleic | atgcagctgaggaacccagaactacatctgggctgcgcgcttgcgcttcgcttcc |
| acid | tggccctcgtttcctgggacatccctggggctagagcactggataatggattggc |
| sequence | tagaacacctcctatggggtggcttcactgggagaggttcatgtgcaacctggac |
| encoding | tgtcaggaagaaccagacagctgcatctccgagaagctctttgaggaaatggccg |
| F | aacgaatggtgactgacggctggaaagatgcaggttacgagtatctgtgtattga |
| | cgattgctggatggccccacaacgggattctgagggaagacttcaggctgatccg |
| | cagcgcttccctcatggcataaggcagctggcaaaccacgtccacagtaaggggc |
| | tcaaattgggaatctacgcggacgtgggcaataagacctgtgccggttttccggg |
| | atcattcgggtattatgacattgacgcccaaacgtttgctgattggggcgttgac |
| | ctgctgaaattcgatggttgctactgtgacagcctcgaaaacctggcagacggct |
| | acaagcatatgtctctcgccctgaataaaaccggtcgggatatcgtatattcctg |
| | cgagtggcctctttacatgtggccatttcagaaaccgaactacacagaaattcgc |
| | cagtattgcaatcattggaggaacttcgctgatatcgatgactcatgggcctcca |
| | taaagagcatcttggactggaccagtcggaatcaggagcgaattgtggatgtcgc |
| | aggccctggaggatggaacgatccagacatgctggtgatcggcaattttggcctc |
| | tcttgggaccagcaggttacccaaatggctctgtgggcaattatggccggccctc |
| | ttttcatgagcaacgatctgcgcgcgatctcaccacaggcaaaggccctgctcca |
| | agacacggacgtgatagccatcaatcaggacccgttgggaaagcagggctaccag |
| | ctgagaaaaggcgacaactttgaggtctgggaaaggcctttgagtggagatgcgt |
| | gggctgtggccattattaatcggcaagagatcggaggtccacgcggttatacaat |
| | tcctgtagcaaagcttggcaagggcgttgcctgtaacccagcttgcttcatcact |
| | cagcttctgccggtgaagagaaaactgggtttctacgaagctaccagcaggctcc |
| | gatcccacatcaaccctacaggaaccgtcctcttgcagctggagaatacgatgca |
| | gacttcactgaaggacctcctgtag (SEQ ID NO: 66) |
| Nucleic | atgcagctgaggaacccagaactacatctgggctgcgcgcttgcgcttcgcttcc |
| acid | tggccctcgtttcctgggacatccctggggctagagcattggacaatgggctggc |
| sequence | caggacacctactatgggctggctccactgggagcgctttatgtgtaacctcgac |
| encoding | tgccaagaggagccagactcatgcatctctgagaagttgttcatggagatggctg |
| 002 | agctgatggtgagcgaagggtggaaggatgcgggctatgagtatctctgtattga |
| | tgactgctggatggctccacagcgcgacagtgaaggccggctccaggccgatcct |
| | cagcggttcccccacggtatcagacaactggcgaattacgtgcactcaaaaggcc |
| | ttaagctgggtatatatgctgatgtgggtaataaaacatgtgcaggcttcccagg |
| | ctcttttgggtactatgacatcgacgcccagacttttgcggactggggcgtggac |
| | ctgctcaagtttgacggatgttactgtgactcccttgagaacctggccgacgggt |
| | acaagcatatgtcactggccctgaatcggacaggccgatccatcgtatactcttg |
| | cgagtggcctctgtacatgtggcccttccagaagcccaactatacagaaatcagg |
| | caatactgcaaccattggcggaacttcgcagacatagacgacagctgggctagca |
| | ttaagtctattctggattggaccagtttcaatcaagaaaggattgtcgatgtcgc |
| | aggcccaggaggttggaatgacccagacatgctcgtgattggaaatttcggtctg |
| | tcatgggaccaacaggtgactcagatggctctgtgggcaatcatggcagcaccac |
| | tgttcatgagcaatgatttgcgacacatctcccctcaggcgaaagcccttctgca |
| | ggataaggacgttatcgccattaaccaggacccgctcggtaagcaagggtaccag |
| | ttgcgccagggagacaatttcgaggtctgggaacgacccctgtctggactcgctt |
| | gggccgtagccgtaattaaccgacaagaaatcggcggaccgcggagctataccat |
| | agctgtcgcctccctcggcgggggcgtagcttgtaacccggcttgtttcataacc |
| | cagctgctgcccgtcaagaggaaactggggctttatgagtggacaagtcggttga |
| | agtctcatattaacccgacagggactgttctcctccagctggaaaacacaatgca |
| | gatgagcctgaaggatctcttgtga (SEQ ID NO: 67) |
| Nucleic | atgcagctgaggaacccagaactacatctgggctgcgcgcttgcgcttcgcttcc |
| acid | tggccctcgtttcctgggacatccctggggctagagcatttggacaatgggctgg |
| sequence | ccaggacacctactatgggctggctccactgggagcgctttatgtgtaacctcga |
| encoding | ctgccaagaggagccagactcatgcatctctgagaagttgttcatggaaatggcc |
| 003 | gagcttatggtctcagaggggtggaaagacgccggatatgagtatctgtgcatcg |
| | acgattgctggatggccccccaacgcgattccgaaggccgcttgcaggctgatcc |
| | gcagcgctttcctcacgggatccgccaattggcaaattacgtgcactcaaagggg |
| | ctcaagttgggcatctacgcagacgtgggaaacaagacatgtgctggatttcccg |
| | ggagtttcggttattatgacattgacgcacagacctttgctgattggggggtcga |
| | cctcctgaagtttgacggttgttattgcgacagccttgaaaatctggccgacggt |
| | tacaaacacatgtcccttgcactgaatagaactggtcggtccatcgtctatagtt |
| | gtgagtggccgctttacatgtggccttttcagaaacccaactacaccgagattcg |
| | gcaatattgcaatcactggcgaaatttcgcagatatcgatgattcttgggctagt |
| | attaaatccatcctggattggacatcattcaaccaggagcgcatcgtggacgttg |
| | ctggacctggcgggtggaatgatccagacatgcttgtgatcggaaacttcggtct |
| | ctcttggaaccagcaagtcactcaaatggcactctgggcaattatggccgccccc |
| | ctctttatgtccaacgatctgaggcatatcagtcctcaggctaaagccctgctgc |
| | aagacaaggatgtgattgctatcaaccaagatccccttggtaaacaggggtacca |
| | gttgcgcaaaggcgacaattttgaggtgtgggaaaggccactttcaggcgatgca |
| | tgggccgttgcaatgatcaacaggcaagaaattggcggacccaggagctatacaa |
| | taccagtggcgtcactgggtaagggagtcgcctgtaaccccgcatgcttcattac |
| | tcaacttctgccagtgaaacgaaaactcggattctatgagtggacctcaagactg |
| | aggtctcatattaacccgacagggactgttctcctccagctggaaaacacaatgc |
| | agatgagcctgaaggatctcttgtga (SEQ ID NO: 68) |
| Nucleic | atgcagctgaggaacccagaactacatctgggctgcgcgcttgcgcttcgcttcc |
| acid | tggccctcgtttcctgggacatccctggggctagagcattggacaatgggctggc |
| sequence | caggacacctactatgggctggctccactgggagcgctttatgtgtaacctcgac |
| encoding | tgccaagaggagccagactcatgcatctctgagaagttgttcatggagatggctg |
| 004 | aacgcatggtttctgagggatggaaagatgcaggctacgagtacctgtgtataga |
| | cgattgttggatggccccacagcgggattcagaaggtagactccaggcggatccc |
| | cagagattcccacatggaatcagacagctggccaattacgtccattccaaaggcc |
| | ttaagttgggtatttacgccgacgtaggcaacaagacttgtgccggatttcccgg |
| | cagtttcggatactatgacattgatgcacagactttcgctgactggggggtggac |
| | ttgctcaaatttgatggctgttattgcgatagcctcgaaaatctggctgatggct |
| | acaagcacatgtcactcgctctcaaccgcactgggcgctctatagtttactcctg |
| | cgagtggcctctgtatatgtggccgttccagaaacccaattacacagaaataagg |
| | cagtattgcaatcactggcgcaactttgctgatattgatgattcctgggcctcca |
| | taaagagtatcttggactggactagtcgcaatcaggaaagaattgtcgacgtcgc |
| | cggaccaggcggatggaatgatcctgatatgctcgtgatcgggaacttcggactc |
| | tcatgggaccagcaggtgacccagatggctagttgggctatcatggccgcccctc |
| | tgtttatgagtaacgacctccgccacatcagcccccaggccaaggcgcttctgca |
| | ggataaagatgtcatcgccatcaaccaagatcccctgggcaaacaaggctatcag |
| | ctgcggaagggagacaattttgaggtgtgggaacgccctttgagcggagacgcct |
| | gggctgtggctattataaatcgccaggagattgggggcccgagaagttatactat |
| | ccccgttgcaagcctgggaaagggcgtcgcctgcaaccctgcctgcttcatcaca |
| | cagctgcttcctgtcaaacgccaattggggttctacaattggacatccagactca |
| | aatctcatattaacccgacagggactgttctcctccagctggaaaacacaatgca |
| | gatgagcctgaaggatctcttgtga (SEQ ID NO: 69) |
| Nucleic | atgcagctgaggaacccagaactacatctgggctgcgcgcttgcgcttcgcttcc |
| acid | tggccctcgtttcctgggacatccctggggctagagcattggacaatgggctggc |
| sequence | caggacacctactatgggctggctccactgggagcgctttatgtgtaacctcgac |
| encoding | tgccaagaggagccagactcatgcatctctgagaagttgttcgaggagatggcag |
| 005 | aacgaatggtgacagatggatggaaggacgctggctacgagtatctgtgcataga |
| | tgattgttggatggcccctcagcgagactcagaggggagactccaggccgacccc |
| | cagcgatttccacacggaatccggcaactggctaaccatgtgcactcaaaagggc |
| | tcaagctgggaatttatgctgacgtcgggaacaaaacttgtgcggggtttcccgg |
| | ctccttcggatattacgacatcgacgcccagactttcgcagactggggtgtggac |
| | ctgcttaagttcgacggctgttactgcgatagtctggaaaacttggctgacggct |
| | ataagcacatgagtctcgccctgaaccgaacaggcagaagcatagtctactcctg |
| | cgaatggccactttacatgtggccattccagaaacctaattataccgagatcaga |
| | caatactgtaaccattggcgaaacttcgccgacattgacgatagttggaagtcaa |
| | tcaagtccatcctggattggacctctaggaaccaggaaaggatcgtggacgtggc |
| | tggacctggcggatggaacgatccagacatgctcgtgataggaaactttggactg |
| | tcatggaatcagcaagtaacacagatggcgctctgggccattatggctgccccct |
| | tgtttatgtctaacgacctgaggcatatctctcctcaagccaaggcactcctgca |
| | ggacaaggacgttatcgccatcaaccaggacccactgggcaagcagggataccag |
| | ctgcggaaaggtgataacttcgaggtctgggagcgaccgctttcaggagacgcct |
| | gggcagttgcaatcatcaacaggcaagaaattggtgggccacggtcttatactat |
| | tcccgtggcttctctcggtaagggcgtcgcctgcaaccccgcctgctttatcacc |
| | caattgttgcccgttaagagaaaactgggattttacgagtggacatccaggctgc |
| | ggtctcatattaacccgacagggactgttctcctccagctggaaaacacaatgca |
| | gatgagcctgaaggatctcttgtga (SEQ ID NO: 70) |
| Nucleic | atgcagctgaggaacccagaactacatctgggctgcgcgcttgcgcttcgcttcc |
| acid | tggccctcgtttcctgggacatccctggggctagagcattggacaatgggctggc |
| sequence | caggacacctactatgggctggctccactgggagcgctttatgtgtaacctcgac |
| encoding | tgccaagaggagccagactcatgcatctctgagaagttgttcgaagagatggctg |
| 006 | aactgatggtgtccgaggggtggaaggatgcagggtacgagtacctttgcataga |
| | cgattgctggatggcaccacagcgggatagtgagggcaggttgcaggcggacccc |
| | caaagatttccacatggcatcagacagctggccaaccacgtgcactctaaaggcc |
| | tgaagctggggatttacgccgatgtcggtaataaaacatgtgctggtttccccgg |
| | tagctttggatactacgacatcgacgcccagacatttgctgattggggagtggac |
| | ctgctcaagttcgacggctgctactgcgattctctggaaaatctggccgatgggt |
| | ataagcacatgagtcttgctcttaatagaaccggacgctctatagtctattcatg |
| | tgagtggccactgtacatgtggccatttcagaaacccaactataccgaaattaga |
| | cagtattgtaaccactggcggaatttcgccgacatagacgatagctggaagagca |
| | tcaagtcaattctcgattggacttcattcaaccaggagcggatcgtcgacgtggc |
| | cggccctgggggctggaatgatccagatatgctggtgatcggaaactttggtctc |
| | tcctggaatcaacaggtcactcagatggccctctgggccattatggcagcaccac |
| | tctttatgagcaacgacctgagacacatttccccacaggccaaggcattgctgca |
| | ggacaaagacgtgatcgccattaaccaggacccactgggcaagcaagggtaccaa |
| | cttaggcagggagataattttgaggtctgggagcgcccactcagcggagacgcct |
| | gggccgttgccatgataaacagacaggagattggcggccccagatcctacacaat |
| | tcctgtcgctagcctgggcaagggggtggcttgtaatcccgcctgctttataacc |
| | cagctgctgccagtgaaacggaaactcgggttctatgaatggacttctaggctca |
| | ggtctcatattaacccgacagggactgttctcctccagctggaaaacacaatgca |
| | gatgagcctgaaggatctcttgtga (SEQ ID NO: 71) |
| Nucleic | atgcagctgaggaacccagaactacatctgggctgcgcgcttgcgcttcgcttcc |
| acid | tggccctcgtttcctgggacatccctggggctagagcattggacaatgggctggc |
| sequence | caggacacctactatgggctggctccactgggagcgctttatgtgtaacctcgac |
| encoding | tgccaagaggagccagactcatgcatctctgagaagttgttcgaggagatggcag |
| 007 | aacgaatggtgacagatggatggaaggacgctggctacgagtatctgtgcataga |
| | tgattgttggatggcccctcagcgagactcagaggggagactccaggccgacccc |
| | cagcgatttccacacggaatccggcaactggctaaccatgtgcactcaaaagggc |
| | tcaagctgggaatttatgctgacgtcgggaacaaaacttgtgcggggtttcccgg |
| | ctccttcggatattacgacatcgacgcccagactttcgcagactggggtgtggac |
| | ctgcttaagttcgacggctgttactgcgatagtctggaaaacttggctgacggct |
| | ataagcacatgagtctcgccctgaaccgaacaggcagaagcatagtctactcctg |
| | cgaatggccactttacatgtggccattccagaaacctaattataccgagatcaga |
| | caatactgtaaccattggcgaaacttcgccgacattgacgatagttggaagtcaa |
| | tcaagtccatcctggattggacctctaggaaccaggaaaggatcgtggacgtggc |
| | tggacctggcggatggaacgatccagacatgctcgtgataggaaactttggactg |
| | tcatgggatcagcaagtaacacagatggcgctctgggccattatggctgccccct |
| | tgtttatgtctaacgacctgaggcatatctctcctcaagccaaggcactcctgca |
| | ggacaaggacgttatcgccatcaaccaggacccactgggcaagcagggataccag |
| | ctgcggaaaggtgataacttcgaggtctgggagcgaccgctttcaggagacgcct |
| | gggcagttgcaatcatcaacaggcaagaaattggtgggccacggtcttatactat |
| | tcccgtggcttctctcggtaagggcgtcgcctgcaaccccgcctgctttatcacc |
| | caattgttgcccgttaagagaaaactgggattttacgaggcgacatccaggctga |
| | agtctcatattaacccgacagggactgttctcctccagctggaaaacacaatgca |
| | gatgagcctgaaggatctcttgtga (SEQ ID NO: 72) |
| Nucleic | atgcagctgaggaacccagaactacatctgggctgcgcgcttgcgcttcgcttcc |
| acid | tggccctcgtttcctgggacatccctggggctagagcatggacaatggattggca |
| sequence | aggacgcctaccatgggctggctgcactgggagcgcttcatgtgcaaccttgact |
| encoding | gccaggaagagccagattcctgcatcagtgagaagctcttcgaggagatggcaga |
| 008 | gagaatggtcaccgacggctggaaggatgcaggttatgagtacctctgcattgat |
| | gactgttggatggctccccaaagagattcagaaggcagacttcaggcagaccctc |
| | agcgctttcctcatgggattcgccagctagctaatcacgttcacagcaaaggact |
| | gaagctagggatttatgcagatgttggaaataaaacctgcgcaggcttccctggg |
| | agttttggatactacgacattgatgcccagacctttgctgactggggagtagatc |
| | tgctaaaatttgatggttgttactgtgacagtttggaaaatttggcagatggtta |
| | taagcacatgtccttggccctgaataggactggcagaagcattgtgtactcctgt |
| | gagtggcctctttatatgtggccctttcaaaagcccaattatacagaaatccgac |
| | agtactgcaatcactggcgaaattttgctgacattgatgattcctgggccagtat |
| | aaagagtatcttggactggacatctagaaaccaggagagaattgttgatgttgct |
| | ggaccagggggttggaatgacccagatatgttagtgattggcaactttggcctca |
| | gctgggaccagcaagtaactcagatggccctctgggctatcatggctgctccttt |
| | attcatgtctaatgacctccgacacatcagccctcaagccaaagctctccttcag |
| | gataaggacgtaattgccatcaatcaggaccccttgggcaagcaagggtaccagc |
| | ttagaaagggagacaactttgaagtgtgggaacgacctctctcaggcgatgcctg |
| | ggctgtagctatcataaaccggcaggagattggtggacctcgctcttataccatc |
| | cccgttgcttccctgggtaaaggagtggcctgtaatcctgcctgcttcatcacac |
| | agctcctccctgtgaaaaggcagctagggttctataactggacttcaaggttaaa |
| | gagtcacataaatcccacaggcactgttttgcttcagctagaaaatacaatgcag |
| | atgtcattaaaagacttactttaa (SEQ ID NO: 73) |
| Nucleic | atgcagctgaggaacccagaactacatctgggctgcgcgcttgcgcttcgcttcc |
| acid | tggccctcgtttcctgggacatccctggggctagagcatggacaatggattggca |
| sequence | aggacgcctCCCatgggctggctgcactgggagcgcttcatgtgcaaccttgact |
| encoding | gccaggaagagccagattcctgcatcagtgagaagctcttcgaggagatggcaga |
| 009 | gagaatggtcaccgacggctggaaggatgcaggttatgagtacctctgcattgat |
| | gactgttggatggctccccaaagagattcagaaggcagacttcaggcagaccctc |
| | agcgctttcctcatgggattcgccagctagctaatcacgttcacagcaaaggact |
| | gaagctagggatttatgcagatgttggaaataaaacctgcgcaggcttccctggg |
| | agttttggatactacgacattgatgcccagacctttgctgactggggagtagatc |
| | tgctaaaatttgatggttgttactgtgacagtttggaaaatttggcagatggtta |
| | taagcacatgtccttggccctgaataagactggcagacccattgtgtactcctgt |
| | gagtggcctctttatatgtggccctttcaaaagcccaattatacagaaatccgac |
| | agtactgcaatcactggcgaaattttgctgacattgatgattcctgggccagtat |
| | aaagagtatcttggactggacatctagaaaccaggagagaattgttgatgttgct |
| | ggaccagggggttggaatgacccagatatgttagtgattggcaactttggcctca |
| | gctgggaccagcaagtaactcagatggccctctgggctatcatggctgctccttt |
| | attcatgtctaatgacctccgacacatcagccctcaagccaaagctctccttcag |
| | gataaggacgtaattgccatcaatcaggaccccttgggcaagcaagggtaccagc |
| | ttagaaagggagacaactttgaagtgtgggaacgacctctctcaggcgatgcctg |
| | ggctgtagctatcataaaccggcaggagattggtggacctcgctcttataccatc |
| | cccgttgcttccctgggtaaaggagtggcctgtaatcctgcctgcttcatcacac |
| | agctcctccctgtgaaaaggcagctagggttctataactggacttcaaggttaaa |
| | gagtcacataaatcccacaggcactgttttgcttcagctagaaaatacaatgcag |
| | atgtcattaaaagacttactttaa (SEQ ID NO: 74) |

In some embodiments, the present disclosure encompasses a gene therapy vector comprising a GLA sequence having 70%, 75%, 80%, 85%, 90%, 95%, 96%, 97%, 98%, 99%, or more than 99% identity to any one of SEQ ID NOs: 18-29, 31, 32, 35-45 and 61-74. In some embodiments, the vector comprises a GLA sequence having between 70% and 100% identity to SEQ ID NOs. 18-29, 31, 32, 35-45, and 61-74. In some embodiments, the vector comprises a GLA sequence having between 75% and 100% identity to SEQ ID NOs. 18-29, 31, 32, 35-45, and 61-74. In some embodiments, the vector comprises a GLA sequence having between 80% and 100% identity to SEQ ID NOs. 18-29, 31, 32, 35-45, and 61-74. In some embodiments, the vector comprises a GLA sequence having between 85% and 100% identity to SEQ ID NOs. 18-29, 31, 32, 35-45, and 61-74. In some embodiments, the vector comprises a GLA sequence having between 90% and 100% identity to SEQ ID NOs. 18-29, 31, 32, 35-45, and 61-74. In some embodiments, the vector comprises a GLA sequence having between 95% and 100% identity to SEQ ID NOs. 18-29, 31, 32, 35-45, and 61-74. In some embodiments, the vector comprises a GLA sequence at least 70% identity to one of SEQ ID NOs. 18-29, 31, 32, 35-45, and 61-74. In some embodiments, the vector comprises a GLA sequence at least 75% identity to one of SEQ ID NOs. 18-29, 31, 32, 35-45, and 61-74. In some embodiments, the vector comprises a GLA sequence at least 80% identity to one of SEQ ID NOs. 18-29, 31, 32, 35-45, and 61-74. In some embodiments, the vector comprises a GLA sequence at least 85% identity to one of SEQ ID NOs. 18-29, 31, 32, 35-45, and 61-74. In some embodiments, the vector comprises a GLA sequence at least 90% identity to one of SEQ ID NOs. 18-29, 31, 32, 35-45, and 61-74. In some embodiments, the vector comprises a GLA sequence at least 95% identity to one of SEQ ID NOs. 18-29, 31, 32, 35-45, and 61-74. In some embodiments, the vector comprises a GLA sequence at least 96% identity to one of SEQ ID NOs. 18-29, 31, 32, 35-45, and 61-74. In some embodiments, the vector comprises a GLA sequence at least 97% identity to one of SEQ ID NOs. 18-29, 31, 32, 35-45, and 61-74. In some embodiments, the vector comprises a GLA sequence at least 98% identity to one of SEQ ID NOs. 18-29, 31, 32, 35-45, and 61-74. In some embodiments, the vector comprises a GLA sequence at least 99% identity to one of SEQ ID NOs. 18-29, 31, 32, 35-45, and 61-74. In some embodiments, the vector comprises a GLA sequence 100% identity to one of SEQ ID NOs. 18-29, 31, 32, 35-45, and 61-74.

In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 35. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 36. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 37. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 38. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 39. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 40. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 41. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 42. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 43. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 44. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 45. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 61. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 62. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 63. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 64. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 65. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 66. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 67. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 68. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 69. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 70. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 71. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 72. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 73. In some embodiments, the present disclosure encompasses a gene therapy vector comprising a GLA sequence having 70%, 75%, 80%, 85%, 90%, 95%, 96%, 97%, 98%, 99%, or more than 99% identity to SEQ ID NO: 74.

In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 7. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 8. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 9. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 10. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 11. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 12. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 13. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 14. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 15. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 16. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 17. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 30. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 33. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 34. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 46. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 47. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 48. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 49. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 50. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 51. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 52. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 53. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 54. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 56. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 57. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 58. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 59. In some embodiments, the present disclosure encompasses a gene therapy vector comprising a nucleic sequence encoding α-GAL enzyme comprising 70%, 75%, 80%, 85%, 90%, 95%, 96%, 97%, 98%, 99%, or more than 99% identity to SEQ ID NO: 60.

In some embodiments, the present disclosure encompasses a gene therapy vector comprising a GLA sequence comprising SEQ ID NO: 18. In some embodiments, the present disclosure encompasses a gene therapy vector comprising a GLA sequence comprising SEQ ID NO: 19. In some embodiments, the present disclosure encompasses a gene therapy vector comprising a GLA sequence comprising SEQ ID NO: 20. In some embodiments, the present disclosure encompasses a gene therapy vector comprising a GLA sequence comprising SEQ ID NO: 21. In some embodiments, the present disclosure encompasses a gene therapy vector comprising a GLA sequence comprising SEQ ID NO: 22. In some embodiments, the present disclosure encompasses a gene therapy vector comprising a GLA sequence comprising SEQ ID NO: 23. In some embodiments, the present disclosure encompasses a gene therapy vector comprising a GLA sequence comprising SEQ ID NO: 24. In some embodiments, the present disclosure encompasses a gene therapy vector comprising a GLA sequence comprising SEQ ID NO: 25. In some embodiments, the present disclosure encompasses a gene therapy vector comprising a GLA sequence comprising SEQ ID NO: 26. In some embodiments, the present disclosure encompasses a gene therapy vector comprising a GLA sequence comprising SEQ ID NO: 27. In some embodiments, the present disclosure encompasses a gene therapy vector comprising a GLA sequence comprising SEQ ID NO: 28. In some embodiments, the present disclosure encompasses a gene therapy vector comprising a GLA sequence comprising SEQ ID NO: 29. In some embodiments, the present disclosure encompasses a gene therapy vector comprising a GLA sequence comprising SEQ ID NO: 31. In some embodiments, the present disclosure encompasses a gene therapy vector comprising a GLA sequence comprising SEQ ID NO: 32. In some embodiments, the present disclosure encompasses a gene therapy vector comprising a GLA sequence comprising SEQ ID NO: 35. In some embodiments, the present disclosure encompasses a gene therapy vector comprising a GLA sequence comprising SEQ ID NO: 36. In some embodiments, the present disclosure encompasses a gene therapy vector comprising a GLA sequence comprising SEQ ID NO: 37. In some embodiments, the present disclosure encompasses a gene therapy vector comprising a GLA sequence comprising SEQ ID NO: 38. In some embodiments, the present disclosure encompasses a gene therapy vector comprising a GLA sequence comprising SEQ ID NO: 39. In some embodiments, the present disclosure encompasses a gene therapy vector comprising a GLA sequence comprising SEQ ID NO: 40. In some embodiments, the present disclosure encompasses a gene therapy vector comprising a GLA sequence comprising SEQ ID NO: 41. In some embodiments, the present disclosure encompasses a gene therapy vector comprising a GLA sequence comprising SEQ ID NO: 42. In some embodiments, the present disclosure encompasses a gene therapy vector comprising a GLA sequence comprising SEQ ID NO: 43. In some embodiments, the present disclosure encompasses a gene therapy vector comprising a GLA sequence comprising SEQ ID NO: 44. In some embodiments, the present disclosure encompasses a gene therapy vector comprising a GLA sequence comprising SEQ ID NO: 45. In some embodiments, the present disclosure encompasses a gene therapy vector comprising a GLA sequence comprising SEQ ID NO: 61. In some embodiments, the present disclosure encompasses a gene therapy vector comprising a GLA sequence comprising SEQ ID NO: 62. In some embodiments, the present disclosure encompasses a gene therapy vector comprising a GLA sequence comprising SEQ ID NO: 63. In some embodiments, the present disclosure encompasses a gene therapy vector comprising a GLA sequence comprising SEQ ID NO: 64. In some embodiments, the present disclosure encompasses a gene therapy vector comprising a GLA sequence comprising SEQ ID NO: 65. In some embodiments, the present disclosure encompasses a gene therapy vector comprising a GLA sequence comprising SEQ ID NO: 66. In some embodiments, the present disclosure encompasses a gene therapy vector comprising a GLA sequence comprising SEQ ID NO: 67. In some embodiments, the present disclosure encompasses a gene therapy vector comprising a GLA sequence comprising SEQ ID NO: 68. In some embodiments, the present disclosure encompasses a gene therapy vector comprising a GLA sequence comprising SEQ ID NO: 69. In some embodiments, the present disclosure encompasses a gene therapy vector comprising a GLA sequence comprising SEQ ID NO: 70. In some embodiments, the present disclosure encompasses a gene therapy vector comprising a GLA sequence comprising SEQ ID NO: 71. In some embodiments, the present disclosure encompasses a gene therapy vector comprising a GLA sequence comprising SEQ ID NO: 72. In some embodiments, the present disclosure encompasses a gene therapy vector comprising a GLA sequence comprising SEQ ID NO: 73. In some embodiments, the present disclosure encompasses a gene therapy vector comprising a GLA sequence comprising SEQ ID NO: 74.

In some embodiments, the present disclosure encompasses a gene therapy vector comprising a nucleic sequence encoding α-GAL enzyme comprising SEQ ID NO: 7. In some embodiments, the present disclosure encompasses a gene therapy vector comprising a nucleic sequence encoding α-GAL enzyme comprising SEQ ID NO: 8. In some embodiments, the present disclosure encompasses a gene therapy vector comprising a nucleic sequence encoding α-GAL enzyme comprising SEQ ID NO: 9. In some embodiments, the present disclosure encompasses a gene therapy vector comprising a nucleic sequence encoding α-GAL enzyme comprising SEQ ID NO: 10. In some embodiments, the present disclosure encompasses a gene therapy vector comprising a nucleic sequence encoding α-GAL enzyme comprising SEQ ID NO: 11. In some embodiments, the present disclosure encompasses a gene therapy vector comprising a nucleic sequence encoding α-GAL enzyme comprising SEQ ID NO: 12. In some embodiments, the present disclosure encompasses a gene therapy vector comprising a nucleic sequence encoding α-GAL enzyme comprising SEQ ID NO: 13. In some embodiments, the present disclosure encompasses a gene therapy vector comprising a nucleic sequence encoding α-GAL enzyme comprising SEQ ID NO: 14. In some embodiments, the present disclosure encompasses a gene therapy vector comprising a nucleic sequence encoding α-GAL enzyme comprising SEQ ID NO: 15. In some embodiments, the present disclosure encompasses a gene therapy vector comprising a nucleic sequence encoding α-GAL enzyme comprising SEQ ID NO: 16. In some embodiments, the present disclosure encompasses a gene therapy vector comprising a nucleic sequence encoding α-GAL enzyme comprising SEQ ID NO: 17. In some embodiments, the present disclosure encompasses a gene therapy vector comprising a nucleic sequence encoding α-GAL enzyme comprising SEQ ID NO: 30. In some embodiments, the present disclosure encompasses a gene therapy vector comprising a nucleic sequence encoding α-GAL enzyme comprising SEQ ID NO: 33. In some embodiments, the present disclosure encompasses a gene therapy vector comprising a nucleic sequence encoding α-GAL enzyme comprising SEQ ID NO: 34. In some embodiments, the present disclosure encompasses a gene therapy vector comprising a nucleic sequence encoding α-GAL enzyme comprising SEQ ID NO: 46. In some embodiments, the present disclosure encompasses a gene therapy vector comprising a nucleic sequence encoding α-GAL enzyme comprising SEQ ID NO: 47. In some embodiments, the present disclosure encompasses a gene therapy vector comprising a nucleic sequence encoding α-GAL enzyme comprising SEQ ID NO: 48. In some embodiments, the present disclosure encompasses a gene therapy vector comprising a nucleic sequence encoding α-GAL enzyme comprising SEQ ID NO: 49. In some embodiments, the present disclosure encompasses a gene therapy vector comprising a nucleic sequence encoding α-GAL enzyme comprising SEQ ID NO: 50. In some embodiments, the present disclosure encompasses a gene therapy vector comprising a nucleic sequence encoding α-GAL enzyme comprising SEQ ID NO: 51. In some embodiments, the present disclosure encompasses a gene therapy vector comprising a nucleic sequence encoding α-GAL enzyme comprising SEQ ID NO: 52. In some embodiments, the present disclosure encompasses a gene therapy vector comprising a nucleic sequence encoding α-GAL enzyme comprising SEQ ID NO: 53. In some embodiments, the present disclosure encompasses a gene therapy vector comprising a nucleic sequence encoding α-GAL enzyme comprising SEQ ID NO: 54. In some embodiments, the present disclosure encompasses a gene therapy vector comprising a nucleic sequence encoding α-GAL enzyme comprising SEQ ID NO: 55. In some embodiments, the present disclosure encompasses a gene therapy vector comprising a nucleic sequence encoding α-GAL enzyme comprising SEQ ID NO: 56. In some embodiments, the present disclosure encompasses a gene therapy vector comprising a nucleic sequence encoding α-GAL enzyme comprising SEQ ID NO: 57. In some embodiments, the present disclosure encompasses a gene therapy vector comprising a nucleic sequence encoding α-GAL enzyme comprising SEQ ID NO: 58. In some embodiments, the present disclosure encompasses a gene therapy vector comprising a nucleic sequence encoding α-GAL enzyme comprising SEQ ID NO: 59. In some embodiments, the present disclosure encompasses a gene therapy vector comprising a nucleic sequence encoding α-GAL enzyme comprising SEQ ID NO: 60.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising an amino acid sequence selected from the group consisting of SEQ ID NOs: 7-17, 30, 33, 34, and 46-60; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO:7; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 8; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 9; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 10; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 11; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 12; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 13; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 14; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 15; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 16; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 17; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 30; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 33; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 34; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 46; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 47; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 48; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 49; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 50; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 51; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 52; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 53; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 54; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 55; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 56; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 57; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 58; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 59; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence encoding α-GAL enzyme comprising SEQ ID NO: 60; d) a poly A; and e) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO:7; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 8; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 9; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 10; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 11; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 12; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 13; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 14; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 15; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 16; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 17; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 30; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 33; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 34; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 46; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 47; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 48; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 49; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 50; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 51; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 52; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 53; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 54; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 55; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 56; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 57; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 58; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 59; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme comprising SEQ ID NO: 60; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO:7; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 8; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 9; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 1; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 11; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 12; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 13; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 14; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 14; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 15; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 16; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 17; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 30; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 33; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 34; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 46; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 47; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 48; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 49; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 50; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 51; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 52; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 53; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 54; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 55; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 56; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 57; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 58; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 59; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding α-GAL enzyme comprising SEQ ID NO: 60; d) a poly A; and e) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO:7; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 8; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 9; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 10; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 11; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 12; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 13; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 14; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 15; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 16; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 17; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 30; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 33; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 34; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 46; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 47; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 48; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 49; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 50; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 51; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 52; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 53; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 54; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 55; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 56; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 57; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 58; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 59; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having muscle or liver tropism); c) a nucleotide sequence encoding α-GAL enzyme comprising SEQ ID NO: 60; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 35; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 36; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 37; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 38; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 39; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 40; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 41; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 42; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 43; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 44; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 45; d) a poly A; and e) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO:35; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 36; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 37; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 38; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 39; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 40; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 41; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 42; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 43; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 44; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 45; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 35; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 36; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 37; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 38; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 39; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 40; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 41; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 42; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 43; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 44; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 45; d) a poly A; and e) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO:35; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 36; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 37; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 38; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 39; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 40; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 41; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 42; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 43; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 44; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 45; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a ubiquitous promoter; c). a nucleotide sequence encoding the sequence set forth in SEQ ID NO: 14; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a ubiquitous promoter; c). a nucleotide sequence encoding the sequence set forth in SEQ ID NO: 10; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a ubiquitous promoter; c). a nucleotide sequence encoding the sequence set forth in SEQ ID NO: 64; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a ubiquitous promoter; c). a nucleotide sequence encoding the sequence set forth in SEQ ID NO: 69; d) a poly A; and e) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme encoding SEQ ID NO: 14; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme encoding SEQ ID NO: 10; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme encoding SEQ ID NO: 64; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence encoding α-GAL enzyme encoding SEQ ID NO: 69; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding the sequence set forth in SEQ ID NO: 14; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding the sequence set forth in SEQ ID NO: 10; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding the sequence set forth in SEQ ID NO: 64; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence encoding the sequence set forth in SEQ ID NO: 69; d) a poly A; and e) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having liver or muscle tropism); c) a nucleotide sequence encoding the sequence set forth in SEQ ID NO: 14; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having liver or muscle tropism); c) a nucleotide sequence encoding the sequence set forth in SEQ ID NO: 10; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having liver or muscle tropism); c) a nucleotide sequence encoding the sequence set forth in SEQ ID NO: 64; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter (e.g., having liver or muscle tropism); c) a nucleotide sequence encoding the sequence set forth in SEQ ID NO: 69; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 35; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 36; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 37; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 38; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 39; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 40; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 41; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 42; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 43; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 44; d) a poly A; and e) a 3′ ITR.

In some embodiments, the present disclosure encompasses a recombinant adeno-associated virus (rAAV) vector packaged in an AAV capsid having broad tissue tropism, where the vector comprises: a) 5′ inverted terminal repeat (ITR); b) a ubiquitous promoter; c). a nucleotide sequence comprising SEQ ID NO: 45; d) a poly A; and e) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 35; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 36; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 37; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 38; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 39; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 40; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 41; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 42; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 43; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 44; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid having broad tissue tropism, said vector comprising a) a 5′ inverted terminal repeat (ITR); b) a ubiquitous promoter; c) a nucleotide sequence comprising SEQ ID NO: 45; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 35; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 36; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 37; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 37; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 38; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 39; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 40; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 41; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 42; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 43; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 44; d) a poly A; and e) a 3′ ITR.

In some embodiments, the present disclosure encompasses a recombinant adeno-associated virus (rAAV) vector packaged in an AAV capsid, where the vector comprises: a) 5′ inverted terminal repeat (ITR); b) a tissue specific promoter (e.g., having muscle or liver tropism); c). a nucleotide sequence comprising SEQ ID NO: 45; d) a poly A; and e) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 35; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 36; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 37; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 38; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 39; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 40; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 41; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 42; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 43; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 44; d) a woodchuck hepatitis virus posttranscriptional regulatory element (WPRE); e) a poly A; and f) a 3′ ITR.

In some embodiments, the present disclosure encompasses a recombinant adeno-associated virus (rAAV) vector packaged in an AAV capsid, said vector comprising a) a 5′ inverted terminal repeat (ITR); b) a tissue specific promoter; c) a nucleotide sequence comprising SEQ ID NO: 45; d) a woodchuck hepatitis virus posttranscriptional regulatory element (WPRE); e) a poly A; and f) a 3′ ITR.

Transgenes delivered by the vector can be introduced into a cell of interest using a variety of methods. For example, either viral or non-viral vectors can be used for the delivery of a transgene of interest. Both viral and non-viral methods of vector delivery are contemplated by the methods provided. Accordingly, in some embodiments, the vector described herein is delivered in a viral vector. In some embodiments, the vector described herein is delivered in a non-viral vector.

A vector as described herein can be introduced into a cell as a part of a viral or non-viral vector molecule having additional sequences, such as, for example, replication origins, promotor and one or more genes. In some embodiments, the vectors can be introduced as naked nucleic acids, as nucleic acid complexed with an agent such as a liposome or a poloxamer, or can be delivered by viruses (e.g., adenovirus, adeno-associated virus (AAV), herpesvirus, retrovirus, lentivirus and integrase defective lentivirus (IDLV). In some embodiments, the vector is introduced using a viral vector.

Various viral vectors are known in the art, and include for example either integrating or non-integrating vectors. In some embodiments, the viral vector is a non-integrating viral vector. Non-integrating viral vectors include, for example non-integrating lentivirus vectors or AAV vectors. Accordingly, in some embodiments, the viral vector is a adeno-associated virus (AAV) vector.

In some embodiments, the AAV vector is modified at one or more regions, such as the AAV capsid. In some embodiments, the rAAV vector is a rAAV9 vector.

In some embodiments, the rAAV vector described herein comprises one or more of: (a) a 5′ inverted terminal repeat (ITR); (b) a ubiquitous promoter sequence; (c) a nucleotide sequence encoding wt-type α-GAL or a variant thereof; (d) a woodchuck hepatitis virus posttranscriptional regulatory element (WPRE), (e) a poly A; and (f) a 3′ ITR sequence.

In various embodiments, a rAAV vector described herein for delivering a transgene (e.g., a gene encoding an alpha-galactosidase (α-GAL) protein) can be packaged using techniques known in the art and as described herein. For example, in some embodiments, rAAV packaging makes use of packaging cells to form virus particles that are capable of infecting a host cell. Such cells include, for example HEK293, HeLa, HEK293T, Sf9 cells or A549 cells, which are used to package adenovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette encoding the protein to be expressed. In this case the protein to be expressed is α-GAL, either wild-type or a modified α-GAL. The missing viral functions can be supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a specificity to a particular tissue type. Prior gene therapy approaches for the treatment of Fabry disease have met with limited success because of reduced tissue tropism for the gene therapy vectors that have previously been used. Unlike the previously used vector designs, the vector design provided here has a wide tissue and cell type distribution once administered to a subject in need thereof. The rAAV vectors described herein have broad tissue distribution and include, for example heart, liver, kidney and gastrointestinal tract.

In some embodiments, a rAAV vector that is able to achieve broad α-GAL enzyme expression upon administration to a subject in need comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme; (d) a woodchuck hepatitis virus posttranscriptional regulatory element (WPRE) having mut6delATG mutation; (e) a bovine growth hormone (BGH) poly A; and (f) a 3′ ITR.

rAAV vector that is able to achieve broad α-GAL enzyme expression upon administration to a subject in need comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme; (d) a bovine growth hormone (BGH) poly A; and (e) a 3′ ITR.

In some embodiments, a rAAV vector that is able to achieve broad α-GAL enzyme expression upon administration to a subject in need is packaged in an AAV9 capsid, the rAAV vector comprises: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding an α-GAL enzyme; (d) a bovine growth hormone (BGH) poly A; (e) a 3′ ITR.

In some embodiments, a rAAV9 vector optionally comprises a woodchuck hepatitis virus posttranscriptional regulatory element (WPRE) (e.g., having mut6delATG mutation) between a nucleotide sequence encoding an α-GAL enzyme and a polyA sequence.

Exemplary sequences for the rAAV are shown in Table 2 below. In some embodiments, the rAAV vector comprises a rAAV vector element comprising a nucleotide sequence having at least 60%, 65%, 70%, 75%, 80%, 85%, 90%, 95% or 99% identity with a vector element sequence shown in the table below. In some embodiments, the rAAV vector comprises a vector element nucleotide sequence identical to a vector element nucleotide sequence shown in the table below.

TABLE 2
Exemplary Vector Component Sequences
5′ITR
Ctgcgcgctcgctcgctcactgaggccgcccgggcaaagcccggg
cgtcgggcgacctttggtcgcccggcctcagtgagcgagcgagcg
cgcagagagggagtggccaactccatcactaggggttcct
(SEQ ID NO: 1)
Chicken-Beta (CB) promoter
gatcttcaatattggccattagccatattattcattggttatata
gcataaatcaatattggctattggccattgcatacgttgtatcta
tatcataatatgtacatttatattggctcatgtccaatatgaccg
ccatgttggcattgattattgactagttattaatagtaatcaatt
acggggtcattagttcatagcccatatatggagttccgcgttaca
taacttacggtaaatggcccgcctggctgaccgcccaacgacccc
cgcccattgacgtcaataatgacgtatgttcccatagtaacgcca
atagggactttccattgacgtcaatgggtggagtatttacggtaa
actgcccacttggcagtacatcaagtgtatcatatgccaagtccg
ccccctattgacgtcaatgacggtaaatggcccgcctggcattat
gcccagtacatgaccttacgggactttcctacttggcagtacatc
tacgtattagtcatcgctattaccatggtcgaggtgagccccacg
ttctgcttcactctccccatctcccccccctccccacccccaatt
ttgtatttatttattttttaattattttgtgcagcgatgggggcg
gggggggggggggggcgcgcgccaggcggggcggggcggggcgag
gggcggggcggggcgaggcggagaggtgcggcggcagccaatcag
agcggcgcgctccgaaagtttccttttatggcgaggcggcggcgg
cggcggccctataaaaagcgaagcgcgcggcgggcgggagtcgct
gcgacgctgccttcgccccgtgccccgctccgccgccgcctcgcg
ccgcccgccccggctctgactgaccgcgttactcccacaggtgag
cgggcgggacggcccttctcctccgggctgtaattagcgcttggt
ttaatgacggcttgtttcttttctgtggctgcgtgaaagccttga
ggggctccgggagggccctttgtgcgggggggagcggctcggggg
gtgcgtgcgtgtgtgtgtgcgtggggagcgccgcgtgcggcccgc
gctgcccggcggctgtgagcgctgcgggcgcggcgcggggctttg
tgcgctccgcagtgtgcgcgaggggagcgcggccgggggcggtgc
cccgcggtgcggggggggctgcgaggggaacaaaggctgcgtgcg
gggtgtgtgcgtgggggggtgagcagggggtgtgggcgcggcggt
cgggctgtaacccccccctgcacccccctccccgagttgctgagc
acggcccggcttcgggtgcggggctccgtacggggcgtggcgcgg
ggctcgccgtgccgggcggggggtggcggcaggtgggggtgccgg
gcggggcggggccgcctcgggccggggagggctcgggggaggggc
gcggcggcccccggagcgccggcggctgtcgaggcgcggcgagcc
gcagccattgccttttatggtaatcgtgcgagagggcgcagggac
ttcctttgtcccaaatctgtgcggagccgaaatctgggaggcgcc
gccgcaccccctctagcgggcgcggggcgaagcggtgcggcgccg
gcaggaaggaaatgggcggggagggccttcgtgcgtcgccgcgcc
gccgtccccttctccctctccagcctcggggctgtccgcgggggg
acggctgccttcgggggggacggggcagggcggggttcggcttct
ggcgtgtgaccggcggctctagagcctctgctaaccatgttcatg
ccttcttctttttcctacagctcctgggcaacgtgctggttattg
tgctgtctcatcattttggcaaagaattcgatatca (SEQ ID
NO: 2)
WT-hGLA1
atgcagctgaggaacccagaactacatctgggctgcgcgcttgcg
cttcgcttcctggccctcgtttcctgggacatccctggggctaga
gcactggacaatggattggcaaggacgcctaccatgggctggctg
cactgggagcgcttcatgtgcaaccttgactgccaggaagagcca
gattcctgcatcagtgagaagctcttcatggagatggcagagctc
atggtctcagaaggctggaaggatgcaggttatgagtacctctgc
attgatgactgttggatggctccccaaagagattcagaaggcaga
cttcaggcagaccctcagcgctttcctcatgggattcgccagcta
gctaattatgttcacagcaaaggactgaagctagggatttatgca
gatgttggaaataaaacctgcgcaggcttccctgggagttttgga
tactacgacattgatgcccagacctttgctgactggggagtagat
ctgctaaaatttgatggttgttactgtgacagtttggaaaatttg
gcagatggttataagcacatgtccttggccctgaataggactggc
agaagcattgtgtactcctgtgagtggcctctttatatgtggccc
tttcaaaagcccaattatacagaaatccgacagtactgcaatcac
tggcgaaattttgctgacattgatgattcctggaaaagtataaag
agtatcttggactggacatcttttaaccaggagagaattgttgat
gttgctggaccagggggttggaatgacccagatatgttagtgatt
ggcaactttggcctcagctggaatcagcaagtaactcagatggcc
ctctgggctatcatggctgctcctttattcatgtctaatgacctc
cgacacatcagccctcaagccaaagctctccttcaggataaggac
gtaattgccatcaatcaggaccccttgggcaagcaagggtaccag
cttagacagggagacaactttgaagtgtgggaacgacctctctca
ggcttagcctgggctgtagctatgataaaccggcaggagattggt
ggacctcgctcttataccatcgcagttgcttccctgggtaaagga
gtggcctgtaatcctgcctgcttcatcacacagctcctccctgtg
aaaaggaagctagggttctatgaatggacttcaaggttaagaagt
cacataaatcccacaggcactgttttgcttcagctagaaaataca
atgcagatgtcattaaaagacttactttaa (SEQ ID NO: 3)
WPREmut6delATG
Aatcaacctctggattacaaaatttgtgaaagattgactggtatt
cttaactttgttgctccttttacgctttgtggatacgctgcttta
ttgcctttgtatcttgctattgcttcccgtttggctttcattttc
tcctccttgtataaatcctggttgctgtctctttttgaggagttg
tggcccgttgtcaggcaacgtggcgtggtgtgcactgtgtttgct
gacgcaacccccactggttggggcattgccaccacctgtcagctc
ctttccgggactttcgctttccccctccctattgccacggcggaa
ctcatcgccgcctgccttgcccgctgctggacaggggctcggctg
ttgggcactgacaattccgtggtgttgtcggggaaatcatcgtcc
tttccttggctgctcgcctgtgttgccacctggattctgcgcggg
acgtccttctgctacgtcccttcggccctcaatccagcggacctt
ccttcccgcggcctgctgccggctctgcggcctcttccgcgtctt
cgccttcgccctcagacgagtcggatctccctttgggccgcctcc
ccgcatc (SEQ ID NO: 4)
bovine growth hormone polyadenylation
signal (bGHpolyA)
Cctagagctcgctgatcagcctcgactgtgccttctagttgccag
ccatctgttgtttgcccctcccccgtgccttccttgaccctggaa
ggtgccactcccactgtcctttcctaataaaatgaggaaattgca
tcgcattgtctgagtaggtgtcattctattctggggggtgggggg
ggcaggacagcaagggggaggattgggaagacaatagcaggcatg
ctggggaa (SEQ ID NO: 5)
3′ITR
Aggaacccctagtgatggagttggccactccctctctgcgcgctc
gctcgctcactgaggccgggcgaccaaaggtcgcccgacgcccgg
gctttgcccgggcggcctcagtgagcgagcgagcgcgcagag
a (SEQ ID NO: 6)

In some embodiments, the present disclosure encompasses a gene therapy vector comprising a GLA gene sequence that is modified. Such modification may be made to improve expression characteristics. Such modifications can include, but are not limited to, insertion of a translation start site (e.g. methionine), addition of a Kozak sequence, insertion of a signal peptide, and/or codon optimization. Accordingly, in some embodiments, the GLA gene is modified to include insertion of a translation start site. In some embodiments, the GLA gene is modified to include the addition of a Kozak sequence. In some embodiments, the GLA gene is modified to comprise a signal peptide. In some embodiments, the GLA gene is codon optimized. In other embodiments, the GLA gene is engineered. In yet other embodiments, the GLA gene is codon optimized and engineered.

In some embodiments, the vector comprises an ID tag, e.g., a stuffer sequence. The purpose of the ID tag includes for example the ability for an artisan to identify the vector. In certain embodiments, the vector comprises woodchuck hepatitis virus posttranscriptional regulatory element (WPRE) element. In some embodiments, the vector comprises woodchuck hepatitis virus post-transcriptional control element (WPRE). Various optimized or variant forms of WPRE are known in the art, and include WPRE3, WPREmut6delATG among others. Other variant WPRE forms include, for example, WPRE2, WPRE_wt (GenBank accession no. J04514); WPRE_wt (GenBank accession no. J02442) and WPREmut6. The WPRE element can comprises a wild-type sequence or a modified WPRE element sequence. Various mutated versions of WPRE are known, and include for example, mut6delATG (SEQ ID NO: 4). In some embodiments, the vector comprises mut6delATG (SEQ ID NO: 4).

The vector described herein comprises one or more promoter sequences. In some embodiments, the promoter sequence is a ubiquitous promoter sequence. Any suitable promoter region or promoter sequence can be used, so long as the promoter region promotes expression of a coding sequence in mammalian cells. In certain embodiments, the promoter region promotes expression of a coding sequence, for example GLA, in mammalian cells. In some embodiments, the promoter controlling the expression of GLA transgene is a ubiquitous promoter. In some embodiments, the ubiquitous promoter is selected from one or more of GAPDH promoter, mini EF1 promoter, CMV promoter EF-1α promoter, PGK promoter, UBC promoter, LSE beta-glucuronidase (GUSB) promoter, or ubiquitous chromatin opening element (UCOE) and/or chicken beta actin promoter. In some embodiments, the ubiquitous promoter comprises ubiquitous promoter comprising a cyto-megalo-virus (CMV) enhancer, chicken beta actin promoter, and a rabbit beta globin intron.

In some embodiments, the vector described herein comprises one or more polyA sequences. In some embodiments, the polyA is selected from human growth hormone polyA (hGHpA), synthetic polyA (SPA), Simian virus 40 late poly A (SV40 pA) and a bovine growth hormone (BGH) poly A.

In some embodiments, the disclosure provides an expression cassette comprising a polynucleotide sequence comprising: (a) a 5′ inverted terminal repeat (ITR); (b) a ubiquitous promoter comprising a cyto-megalo-virus (CMV) enhancer, chicken beta actin promoter, and a rabbit beta globin intron; (c) a nucleotide sequence encoding α-GAL enzyme; (d) optionally a woodchuck hepatitis virus posttranscriptional regulatory element (WPRE) comprising the mut6delATG mutation; (e) a bovine growth hormone (BGH) poly A; and (f) a 3′ ITR. In some embodiments, the elements in the expression cassette above are present in 5′ to 3′ order. In various embodiments, one or more of (a) to (f) are operably linked in 5′ to 3′ order.

In some embodiments, the vector is introduced into a cell. Accordingly, in some embodiments, a cell is provided, said cell comprising the vector described herein. In some embodiments, a cell is in vitro, in situ or in vivo. Accordingly, in some embodiments, the cell comprising the vector described herein is in vitro. In some embodiments, the cell comprising the vector described herein is in situ. In some embodiments, the cell comprising the vector described herein is in vivo.

### Pharmaceutical Compositions

Exemplary pharmaceutical compositions comprising the vectors described herein are detailed below.

Pharmaceutical acceptable carriers are determined in part by the particular composition being administered, as well as by the particular method used to administer the compositions. Accordingly, there is a wide variety of suitable formulations of pharmaceutical compositions available.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol, or the like, and combinations thereof. In addition, the compositions may contain minor amounts of auxiliary substances, such as wetting or emulsifying agents, pH buffering agents, stabilizing agents or other reagents that enhance the effectiveness of the pharmaceutical composition.

### Methods of Treatment

The vectors of the present disclosure can be used to treat a subject who has Fabry disease. Accordingly, the vectors of the present disclosure can be used to treat a subject who has Fabry disease, and as such reduce one or more symptoms associated with the disease. In some embodiments, the vectors of the present disclosure can be used to treat a subject who has reduced expression or no expression of α-GAL.

Non-limiting examples of Fabry symptoms include neuropathic pain, hypohidrosis or anhidrosis, exercise intolerance, abdominal cramps, diarrhea, angiokeratoma, verticillata, tinnitus, proteinuria, chronic kidney disease, hypertension, coronary insufficiency, AV conduction disturbances, arrhythmias and valvular malfunction, left ventricular hypertrophy, seizure and stroke.

In some embodiments, the vectors provided herein are used as a prophylactic treatment in a subject who has Fabry disease. Prophylactic treatment may be administered, for example, to a subject who is not yet ill, but who is susceptible to, or otherwise at risk of, a particular biological condition, including Fabry disease (e.g., the subject may have mutations that cause Fabry disease but is asymptomatic or the status of mutations that cause Fabry disease is unknown). In some embodiments, therapeutic treatment may be administered, for example, to a subject already suffering from Fabry disease in order to improve or stabilize the subject's condition (e.g., a patient already presenting symptoms of Fabry disease).

In some embodiments, the rAAV vector remains episomal following administration to a subject in need thereof. In some embodiments, the rAAV vector does not remain episomal following administration to a subject in need thereof. For example, in some embodiments, the rAAV vector integrates into the genome of the subject. Such integration can be achieved, for example, by using various gene-editing technologies, such as, zinc finger nucleases (ZFNs), Transcription activator-like effector nucleases (TALENS), ARCUS genome editing, and/or CRISPR-Cas systems.

In some embodiments, a pharmaceutical composition comprising a rAAV vector described herein is used to treat subjects in need thereof. The pharmaceutical composition containing a rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal administration. In some embodiments, the rAAV vector is administered intravenously. In some embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle.

In some embodiments, functional α-GAL is detectable in plasma or serum of the subject at about 2 to 15 weeks post administration of the rAAV vector. In some embodiments, functional α-GAL is detectable in plasma or serum of the subject at about 2 weeks. In some embodiments, functional α-GAL is detectable in plasma or serum of the subject at about 3 weeks. In some embodiments, functional α-GAL is detectable in plasma or serum of the subject at about 4 weeks. In some embodiments, functional α-GAL is detectable in plasma or serum of the subject at about 5 weeks. In some embodiments, functional α-GAL is detectable in plasma or serum of the subject at about 6 weeks. In some embodiments, functional α-GAL is detectable in plasma or serum of the subject at about 7 weeks. In some embodiments, functional α-GAL is detectable in plasma or serum of the subject at about 8 weeks. In some embodiments, functional α-GAL is detectable in plasma or serum of the subject at about 9 weeks. In some embodiments, functional α-GAL is detectable in plasma or serum of the subject at about 10 weeks. In some embodiments, functional α-GAL is detectable in plasma or serum of the subject at about 11 weeks. In some embodiments, functional α-GAL is detectable in plasma or serum of the subject at about 12 weeks. In some embodiments, functional α-GAL is detectable in plasma or serum of the subject at about 13 weeks. In some embodiments, functional α-GAL is detectable in plasma or serum of the subject at about 14 weeks. In some embodiments, functional α-GAL is detectable in plasma or serum of the subject at about 15 weeks. In some embodiments, functional α-GAL is detectable in hepatocytes of the subject at about 2 to 15 weeks post administration of the rAAV vector.

In some embodiments, functional α-GAL is detectable in plasma of the subject at least 3 months, 6 months, 12 months, 2 years, 3 years, 4 years, 5 years, 6 years, 7 years, 8 years, 9 years, 10 years, 15 years or 20 years after administration of the rAAV vector. Accordingly, in some embodiments, functional α-GAL is detectable in plasma or serum of the subject at least 3 months after administration of the rAAV vector. In some embodiments, functional α-GAL is detectable in plasma or serum of the subject at least 6 months after administration of the rAAV vector. In some embodiments, functional α-GAL is detectable in plasma or serum of the subject at least 12 months after administration of the rAAV vector. In some embodiments, functional α-GAL is detectable in plasma or serum of the subject at least 2 years after administration of the rAAV vector. In some embodiments, functional α-GAL is detectable in plasma or serum of the subject at least 3 years after administration of the rAAV vector. In some embodiments, functional α-GAL is detectable in plasma or serum of the subject at least 4 years after administration of the rAAV vector. In some embodiments, functional α-GAL is detectable in plasma or serum of the subject at least 5 years after administration of the rAAV vector. In some embodiments, functional α-GAL is detectable in plasma or serum of the subject at least 6 years after administration of the rAAV vector. In some embodiments, functional α-GAL is detectable in plasma or serum of the subject at least 7 years after administration of the rAAV vector. In some embodiments, functional α-GAL is detectable in plasma or serum of the subject at least 8 years after administration of the rAAV vector. In some embodiments, functional α-GAL is detectable in plasma or serum of the subject at least 9 years after administration of the rAAV vector. In some embodiments, functional α-GAL is detectable in plasma or serum of the subject at least 10 years after administration of the rAAV vector. In some embodiments, functional α-GAL is detectable in plasma or serum of the subject for the remainder of the subject's life following administration of the rAAV vector.

In some embodiments, the administered rAAV comprising GLA results in the production of active α-GAL to the same extent as found following administration of purified GLA protein delivered intravenously. In some embodiments, the administered rAAV comprising GLA results in production of a greater amount of active α-GAL as compared to administration of purified α-GAL protein delivered intravenously.

In some embodiments, the administered rAAV comprising GLA results in the reduction of globotriaosylceramide (GB3) in the subject. In some embodiments, the administered rAAV comprising GLA reduces GB3 in the subject by about 95%, 90%, 85%, 80%, 75%, 70%, 65%, 60%, 55%, 50%, 45%, 40%, 35%, 30%, 25%, 20%, 15%, or about 10% in comparison to the subject's baseline GB3 level prior to administering the rAAV comprising GLA. Accordingly, in some embodiments, the administered rAAV comprising GLA reduces GB3 in the subject by about 95%. In some embodiments, the administered rAAV comprising GLA reduces GB3 in the subject by about 90%. In some embodiments, the administered rAAV comprising GLA reduces GB3 in the subject by about 85%. In some embodiments, the administered rAAV comprising GLA reduces GB3 in the subject by about 80%. In some embodiments, the administered rAAV comprising GLA reduces GB3 in the subject by about 75%. In some embodiments, the administered rAAV comprising GLA reduces GB3 in the subject by about 70%. In some embodiments, the administered rAAV comprising GLA reduces GB3 in the subject by about 65%. In some embodiments, the administered rAAV comprising GLA reduces GB3 in the subject by about 60%. In some embodiments, the administered rAAV comprising GLA reduces GB3 in the subject by about 55%. In some embodiments, the administered rAAV comprising GLA reduces GB3 in the subject by about 50%. In some embodiments, the administered rAAV comprising GLA reduces GB3 in the subject by about 45%. In some embodiments, the administered rAAV comprising GLA reduces GB3 in the subject by about 40%. In some embodiments, the administered rAAV comprising GLA reduces GB3 in the subject by about 35%. In some embodiments, the administered rAAV comprising GLA reduces GB3 in the subject by about 30%. In some embodiments, the administered rAAV comprising GLA reduces GB3 in the subject by about 25%. In some embodiments, the administered rAAV comprising GLA reduces GB3 in the subject by about 20%. In some embodiments, the administered rAAV comprising GLA reduces GB3 in the subject by about 15%. In some embodiments, the administered rAAV comprising GLA reduces GB3 in the subject by about 10%.

In some embodiments, the administered rAAV comprising GLA reduces GB3 in the subject for at least about 2 weeks, 1 month, 2 months, 3 months, 4 months, 5 months, 6 months, 12 months, 1 year, 2 years, 3 years, 4 years, 5 years, or more than 5 years.

In some embodiments, following administration of the AAV vector to the subject the levels of functional α-GAL detectable in the circulation are between about 2 and 100 fold or higher than 10 fold, higher than 20 fold, higher than 30 fold, higher than 40 fold, higher than 50 fold, higher than 60 fold, higher than 70 fold, higher than 80 fold, higher than 90 fold, higher than 95 fold, or 100 fold or more greater than the amount of functional α-GAL detectable in the subject before administration of the rAAV comprising GLA transgene.

In some embodiments, following administration of the AAV vector to the subject the levels of detectable active α-GAL meets or exceeds human therapeutic level, i.e., level of α-GAL considered to be normal circulating level in humans (e.g., 5-9 nmol/hour/ml). In some embodiments, the levels of active α-GAL post administration of the rAAV vector is about between 2 and 35 times or greater than 35 times, greater than 40 times, greater than 45 times, greater than 50 times, greater than 55 times, greater than 60 times, greater than 65 times, greater than 70 times greater than 75 times greater than 80 times greater than 85 times greater than 90 times, greater than 95 times, or greater than 100 the human therapeutic level. In some embodiments, the levels of active α-GAL post administration is about 2 times the human therapeutic level. In some embodiments, the levels of active α-GAL post administration is about 3 times the human therapeutic level. In some embodiments, the levels of active α-GAL post administration is about 4 times the human therapeutic level. In some embodiments, the levels of active α-GAL post administration is about 5 times the human therapeutic level. In some embodiments, the levels of active α-GAL post administration is about 6 times the human therapeutic level. In some embodiments, the levels of active α-GAL post administration is about 7 times the human therapeutic level. In some embodiments, the levels of active α-GAL post administration is about 8 times the human therapeutic level. In some embodiments, the levels of active α-GAL post administration is about 9 times the human therapeutic level. In some embodiments, the levels of active α-GAL post administration is about 10 times the human therapeutic level. In some embodiments, the levels of active α-GAL post administration is about 11 times the human therapeutic level. In some embodiments, the levels of active α-GAL post administration is about 15 times the human therapeutic level. In some embodiments, the levels of active α-GAL post administration is about 20 times the human therapeutic level. In some embodiments, the levels of active α-GAL post administration is about 25 times the human therapeutic level. In some embodiments, the levels of active α-GAL post administration is about 30 times the human therapeutic level. In some embodiments, the levels of active α-GAL post administration is about 35 times the human therapeutic level. In some embodiments, the levels of active α-GAL post administration is about 40 times the human therapeutic level. In some embodiments, the levels of active α-GAL post administration is about 45 times the human therapeutic level. In some embodiments, the levels of active α-GAL post administration is about 50 times the human therapeutic level. In some embodiments, the levels of active α-GAL post administration is about 55 times the human therapeutic level. In some embodiments, the levels of active α-GAL post administration is about 60 times the human therapeutic level. In some embodiments, the levels of active α-GAL post administration is about 65 times the human therapeutic level. In some embodiments, the levels of active α-GAL post administration is about 70 times the human therapeutic level. In some embodiments, the levels of active α-GAL post administration is about 75 times the human therapeutic level. In some embodiments, the levels of active α-GAL post administration is about 80 times the human therapeutic level. In some embodiments, the levels of active α-GAL post administration is about 85 times the human therapeutic level. In some embodiments, the levels of active α-GAL post administration is about 90 times the human therapeutic level. In some embodiments, the levels of active α-GAL post administration is about 95 times the human therapeutic level. In some embodiments, the levels of active α-GAL post administration is about 100 times the human therapeutic level. In some embodiments, the levels of active α-GAL post administration is about 200 times the human therapeutic level. In some embodiments, the levels of active α-GAL post administration is about 300 times the human therapeutic level. In some embodiments, the levels of active α-GAL post administration is about 400 times the human therapeutic level. In some embodiments, the levels of active α-GAL post administration is about 500 times or greater than 500 times the human therapeutic level.

Thus, administration of rAAV vector comprising a GLA transgene results in sustained robust expression in comparison to a single administration of purified α-GAL to a subject in need.

In some embodiments, the rAAV vector comprising a GLA transgene is delivered as a single dose per subject. In some embodiments, the subject is delivered the minimal effective dose (MED). As used herein, MED refers to the rAAV GLA vector dose required to achieve α-GAL activity resulting in reduced GB3 levels in a subject.

The vector titer is determined on the basis of the DNA content of the vector preparation. In some embodiments, quantitative PCR or optimized quantitative PCR is used to determine the DNA content of the rAAV GLA vector preparations. In one embodiment, the dosage is about 1×10¹¹ genome copies (GC)/kg body weight to about 1×10¹³ GC/kg, inclusive of endpoints.

The dosages to achieve therapeutic benefit in a subject in need thereof are lower than those achieved using other kinds of rAAV capsids, such as AAV1, AAV2, AAV3, AAV4, AAV5, AAV6, AAV7, and/or AAV8 which include a liver specific promoter. In some embodiments, a rAAV gene therapy vector expressing an alpha-galactosidase protein as described herein is administered to a subject at a lower or an equivalent dose used in case of a gene therapy vector which includes a liver specific promoter; however, surprisingly exhibits higher serum and tissue exposure.

In some embodiments, the rAAV GLA vector compositions can be formulated in dosage units to contain an amount of replication-defective virus that is in the range of about 1.0×10⁹ GC to about 1.0×10¹⁵ GC. As used herein, the term “dosage” can refer to the total dosage delivered to the subject in the course of treatment, or the amount delivered in a single (of multiple) administration.

In some embodiments, the dosage is sufficient to decrease plasma GB3 levels in the patient by 25% or more. In some embodiments, rAAV expressing α-GAL is administered in combination with one or more therapies for the treatment of Fabry disease.

### Combination Therapy

The compositions and methods of the invention can also be used in conjunction with other remedies known in the art that are used to treat Fabry disease or its complications, including but not limited to: ERT (e.g., agalsidase beta), pain relief medications (e.g., lidocaine, diphenylhydantoin, carbamazepine, gabapentin, phenytoin, neurotropin, opioids); dyspepsia treatment (e.g., metoclopramide, H-2 blockers), vitamin D replacements etc, beta blockers (metoprolol, Acebutolol, bisoprolol, atenolol, propranolol, etc) anti-coagulation treatment (Heparin, warfarin, Apixaban, Rivaroxaban).

The compositions and methods of the invention can also be used in conjunction with other forms of treatment including but not limited to: physical exercise (e.g. dialysis, kidney transplantation); dietary salt restriction, fiber intake, installation of a pacemaker, and cardiac transplantation.

Production of rAAV Viral Vectors

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

In some embodiments, the expression cassette flanked by ITRs and rep/cap genes are introduced into a desired cell or cell line by infection with baculovirus-based vectors.

In some embodiments, the expression cassette flanked by ITRs and rep/cap genes are introduced into insect cells by infection with baculovirus-based vectors. For reviews on these production systems, see generally, e.g., Zhang et al, 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin Biotechnol. 99: 119-145; Buning et al, 2008, “Recent developments in adeno-associated virus vector technology,” J. Gene Med 10:717-733; and the references cited below, each of which is incorporated herein by reference in its entirety.

The methods used to construct a vector as described herein are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol, 70:520-532 and U.S. Pat. No. 5,478,745.

Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In one embodiment, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

In certain embodiments, the rAAV expression cassette, the vector (such as rAAV vector), the virus (such as rAAV), the production plasmid comprises AAV inverted terminal repeat sequences, a codon optimized nucleic acid sequence that encodes an α-GAL polypeptide, and expression control sequences that direct expression of the encoded proteins are present in a host cell. In other embodiments, the rAAV expression cassette, the virus, the vector (such as rAAV vector), the production plasmid further comprise one or more of an intron, a Kozak sequence, a polyA, posttranscriptional regulatory elements and others. In one embodiment, the post-transcriptional regulatory element is Woodchuck Hepatitis Virus (WHP) Posttranscriptional Regulatory Element (WPRE). In various embodiments, the nucleic acid sequence comprises a signal peptide upstream of the transgene that encodes an α-GAL polypeptide. In some embodiments, a signal peptide is at the N-terminus of an α-GAL polypeptide. In some embodiments, a signal peptide is at the C-terminus of an α-GAL polypeptide.

Various methods are known in the art relating to the production and purification of AAV vectors. See, e.g., Mizukami, Hiroaki, et al. A Protocol for AAV vector production and purification; U.S. Patent Publication Numbers US20070015238 and US20120322861. For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV can be packaged and subsequently purified.

In some embodiments, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprise rep and/or cap genes that encode the Rep protein and/or Cap proteins for use in a method described herein. In some embodiments, the packaging is performed in vitro.

In some embodiments, a plasmid containing comprising the gene of interest is combined with one or more helper plasmids, e.g., that contain a rep gene of a first serotype and a cap gene of the same serotype or a different serotype, and transfected into helper cells such that the rAAV is packaged.

In some embodiments, the one or more helper plasmids include a first helper plasmid comprising a rep gene and a cap gene, and a second helper plasmid comprising one or more of the following helper genes: Ela gene, Elb gene, E4 gene, E2a gene, and VA gene. For clarity, helper genes are genes that encode helper proteins Ela, Elb, E4, E2a, and VA. In some embodiments, the cap gene is modified such that one or more of the proteins VP1, VP2 and VP3 do not get expressed. In some embodiments, the cap gene is modified such that VP2 does not get expressed. Methods for making such modifications are known in the art (Lux et al. (2005), J Virology, 79: 11776-87).

Helper plasmids, and methods of making such plasmids, are generally known in the art and generally commercially available (see, e.g., pDF6, pRep, pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adeno associated Virus Vectors, Human Gene Therapy, Vol. 9, 2745-2760; Kem, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005).

### EXAMPLES

Exemplary features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

The data in the Examples below were generated with GLA transgenes comprising signal peptide sequence of SEQ ID NO:77, which encode α-GAL enzymes comprising signal peptide sequence of SEQ ID NO: 76.

### Example 1. Design and Purification Viral Vectors for Expression of α-GAL Enzyme

This Example summarizes the design of exemplary viral vectors encompassed by the present disclosure.

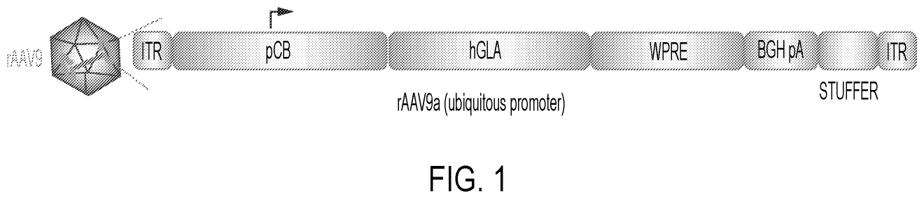

A recombinant adeno-associated virus 9 (rAAV9) was developed to express wild type human α-GAL or α-GAL variants (e.g., amino acid sequences shown in Table 1) under the control of a ubiquitous promoter, in a viral vector. A WPRE element was linked to the 3′ end of the wild type GLA transgene to increases transgene expression and improve mRNA stability. A bovine growth hormone polyA tail was appended to the 3′ end of the WPRE element. The DNA construct of promoter-GLA-WPRE-BGHpA was integrated between the inverted terminal repeats of a circular plasmid vector. FIG. 1 shows an exemplary rAAV9 vector construct.

rAAV vectors were encapsulated using the AAV2 inverted terminal repeats and rep sequences using methods in the art. The rAAV9 stocks were produced using HEK-293T cells by the adenovirus free, triple-plasmid co-transfection method and purified using cesium chloride ultracentrifugation. Titers of v.g. particle number were determined by quantitative PCR.

Purified rAAV9 virus suspension were diluted in the formulation buffer consisting of 1.5 mM KH₂PO₄ (Potassium dihydrogen phosphate), 2.7 mM KCl (Potassium chloride), 8.1 mM Na₂HPO₄ (Di-sodium hydrogen phosphate), 136.9 mM NaCl (Sodium chloride) and 0.001% Pluronic F-68. Null vector with rAAV9 capsid (rAAV9-null) were used as controls.

### Example 2. Serum Stability of α-GAL Following Administration of rAAV9

This Example shows that rAAV9-delivered GLA transgene provided α-GAL protein expression in serum for at least 12 weeks following administration of vector into mice.

First, rAAV9 vector was produced and purified as described in Example 1 above. Next, 6-9 months old male GLAko mice were administered the purified rAAV9 vectors encoding α-GAL intravenously (IV) at two different doses, 2.5×10¹¹, 6.25×10¹² vg/kg and the mice were followed for 12 weeks. Null vector rAAV9-null was administered to a group of GLAko mice at 6.25×10¹² vg/kg as a negative control. Serum was collected at multiple time-points during the study as well as at the end of 12-weeks. In a subset of animals, 1 mg/kg dose of α-GAL ERT was administered 24 hours before sacrifice and collection of tissues to be used as positive control. Serum was collected 1 hour after administration of α-GAL ERT to capture the Cₘₐₓ levels of circulating enzyme.

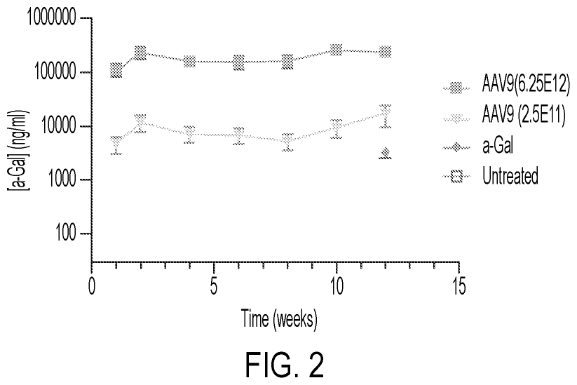

The quantity of α-GAL in the serum was measured using ELISA. Briefly, a high binding MSD black plate (MSD, catalog #L15XB) was coated with a polyclonal sheep anti-human α-GLA capture antibody (R&D systems, catalog #AF6146) in 0.2 M sodium carbonated-bicarbonate buffer (Thermoscientific, catalog #28382) overnight at 4° C. Then, the coated plate was washed three times with wash buffer containing D′PBS and 0.05% Tween-20. The plate was blocked with blocking buffer (3% BSA in PBS) for 1 hour before samples or purified α-GLAprotein standard in diluent buffer (1% BSA in PBS) were added. Binding was carried for 1 hour with a low speed shaking and washed three times with wash buffer. Then, polyclonal rabbit anti-human α-GLA (Novus Biologics, catalog #H00002717-D01P) in diluent buffer was added and incubated for 1 hour before washing three times with wash buffer and adding a detection antibody, sulfo-tag goat anti-rabbit antibody (MSD, catalog #R32AB-1), for 1 hour at room temperature. The plate was read with 1× read buffer (MSD, catalog #R92TC-1) in the MSD Sector Imager S600 system. Using a standard curve, final values of α-GAL concentration were calculated. Tissue α-GAL protein concentration was normalized by total protein concentration determined by BCA assay. None of antibodies used in this assay recognize mouse α-GAL protein. A rapid elevation of α-GAL was observed in serum at 2 weeks post vector administration, reaching more than 4500-fold of normal in the similar high dose group for rAAV9-WT at 12 weeks. It was observed that even at the lowest dose, rAAV9-WT resulted in a steady level of serum α-GAL enzyme that was higher than the Cₘₐₓ of the positive control α-GAL ERT (FIG. 2).

At 12-weeks post administration, multiple organs including liver, kidney, heart and GI tract were harvested for further evaluation. Fixation with 10% NBF was carried out for histology evaluation. For physiology, tissues were snap frozen and stored at −80° C.

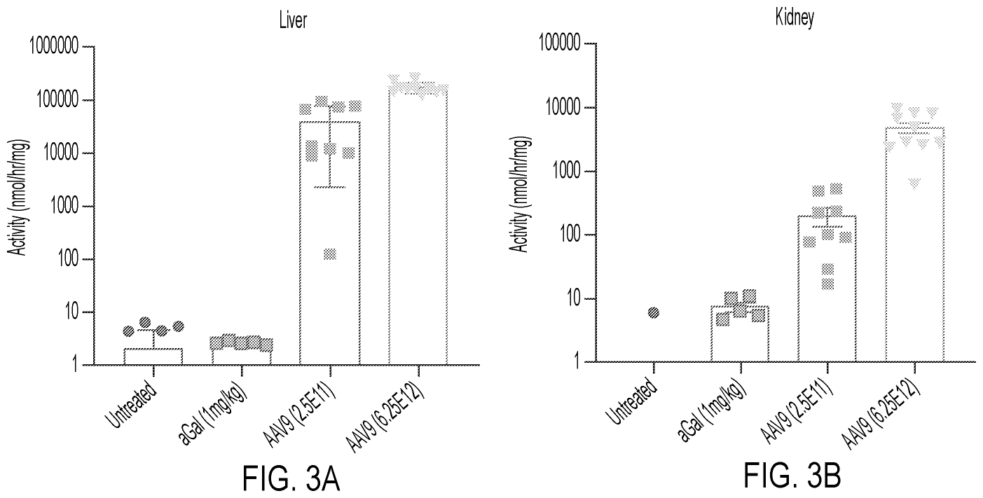

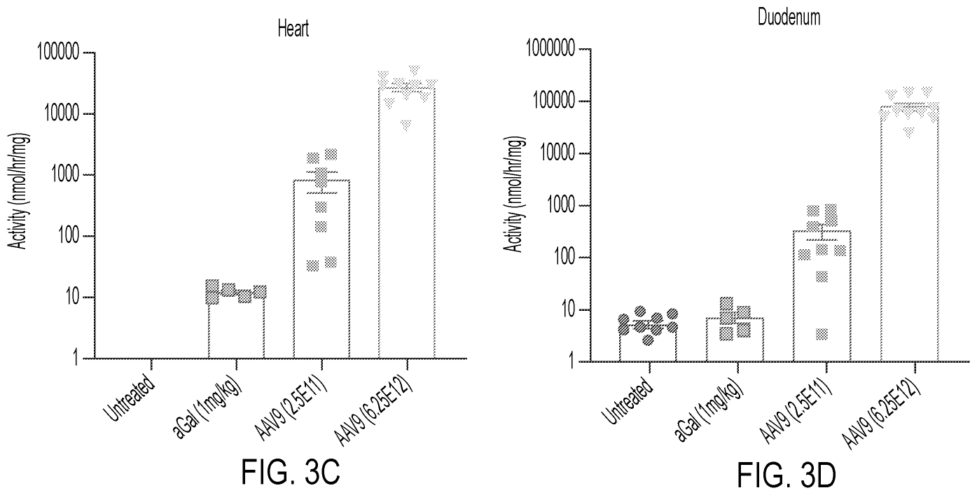

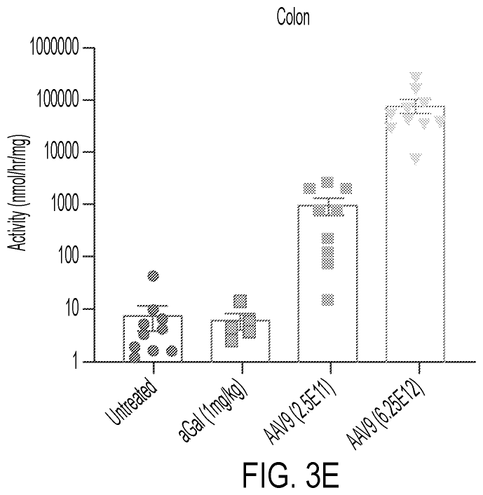

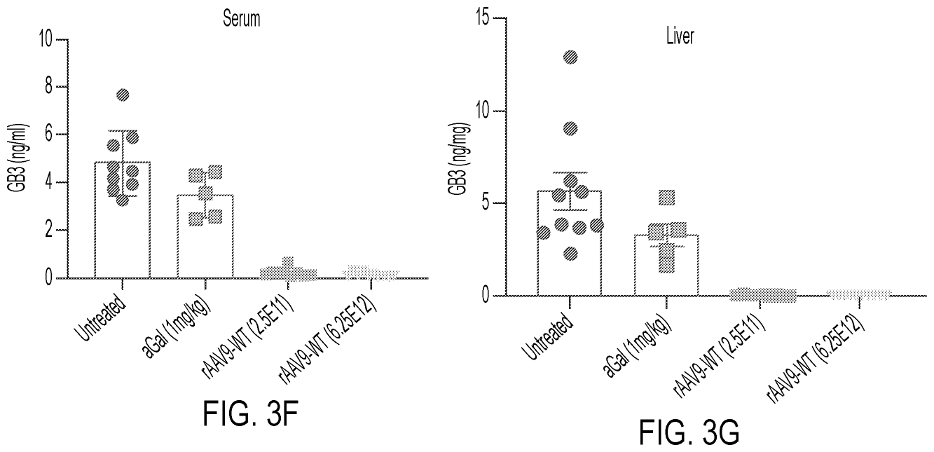

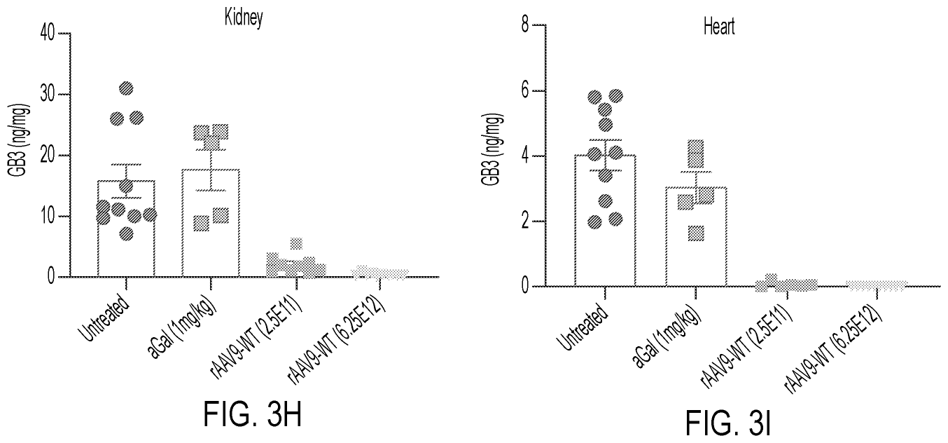

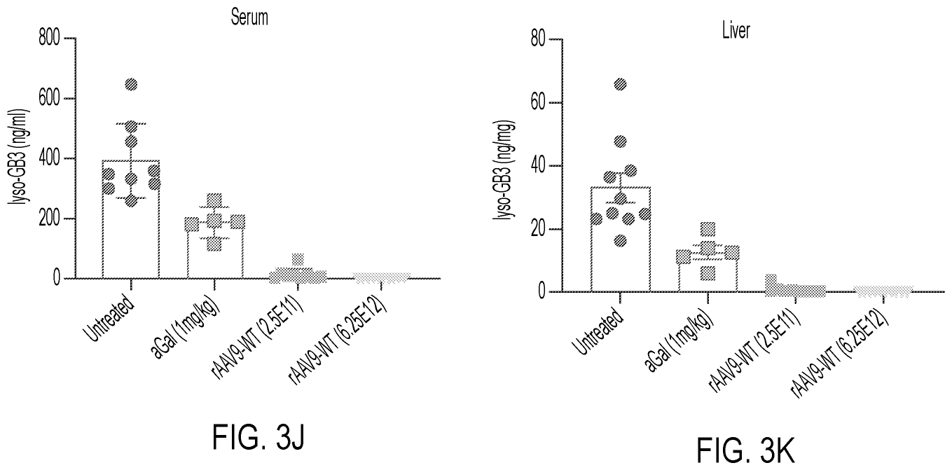

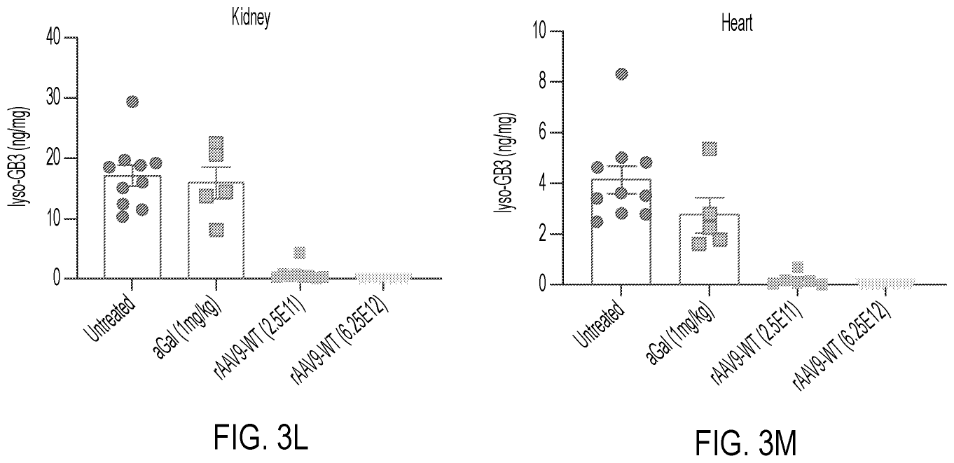

Dose-dependent sustained supraphysiological levels of α-GAL exposure was observed in peripheral organs including liver (FIG. 3A), kidneys (FIG. 3B), heart (FIG. 3C) and GI tract (FIG. 3D-3E), resulting in concomitant reduction in GB3 and lysoGb3 accumulation in these animals (FIG. 3F-3M). It was further observed that the higher dose of rAAV9-WT, reduced the GB3 and lysoGb3 levels in serum and kidneys by >95% (FIG. 3F, FIG. 3H, FIG. 3J, FIG. 3L) and in the liver and heart by >99% (FIG. 3G, FIG. 3I, FIG. 3K, FIG. 3M). Similarly, lysoGb3 levels in all tissues tested were reduced by >99% after the high dose injections of rAAV9-WT. In contrast, as seen from FIG. 3F-3M, in mice receiving α-GAL protein, there was moderate reduction in substrate levels in serum and liver but not in the kidneys and heart.

It was overall observed that treatment with rAAV9-WT encoding α-GAL improved GB3 and lysoGb3 clearance in various tissues. Further, the expression of α-GAL was sustained for at least 12 weeks.

### Example 3. Effect of rAAV9-WT Treatment of G3Stg/GLAko Mice

This Example shows in vivo effects of rAAV9-WT encoding α-GAL expression in various tissues in a severe Fabry-disease mouse model that has significantly higher substrate levels in various tissues than the GLAko mice. The data from this Example demonstrated that rAAV9-WT delivered GLA transgene reduced GB3 accumulation associated with Fabry disease.

First, rAAV9-WT vector was produced and purified as described in Example 1 above.

Next, G3Stg/GLA knockout mice were generated by crossing GLA knockout mice, which have a C57BL/6 background, with GB3 synthase transgenic mice. The founders were purchased from Jackson labs and the breeding colony was maintained at Taconic Biosciences. The G3Stg/GLA knockout mice have substantial deposition of GB3 substrates in visceral organs which results in severe renal, GI and neuropathic phenotypes, including albuminuria, reduced kidney osmolarity, delayed colonic propulsion and loss of thermosensitivity, which reflects several Fabry disease manifestations (Taguchi et. al., 2013). C57BL/6 mice (Charles River Laboratories) were used as wild type control for this experiment. Animals were maintained in a controlled environment on a 12 h dark/12 h light cycle (lights on at 7:00 am) with no more than 4 mice per cage in a ventilated cage rack system at Melior Discoveries and fed standard rodent chow and water ad libitum.

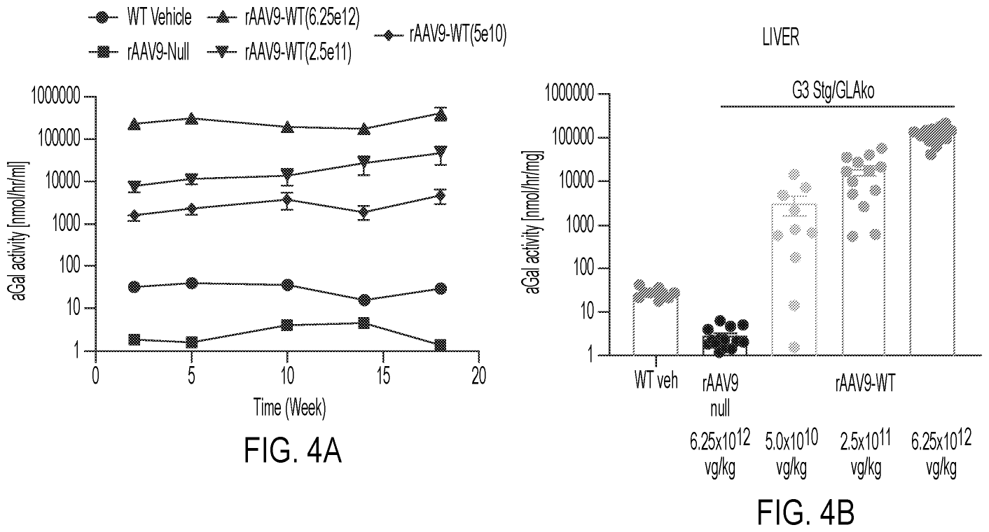

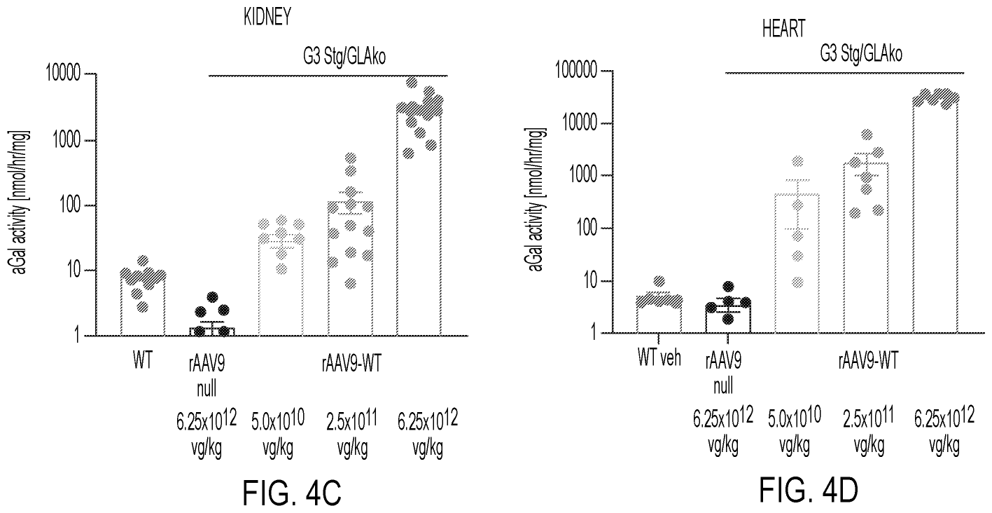

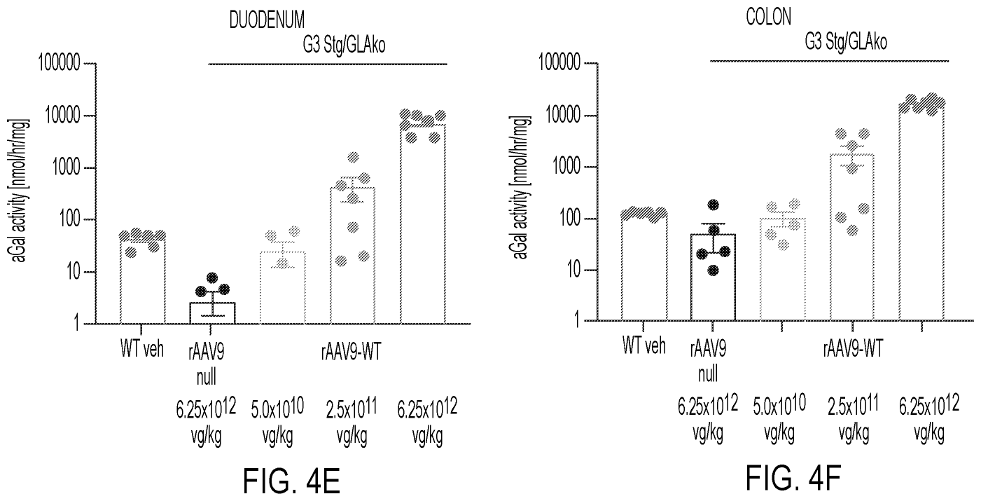

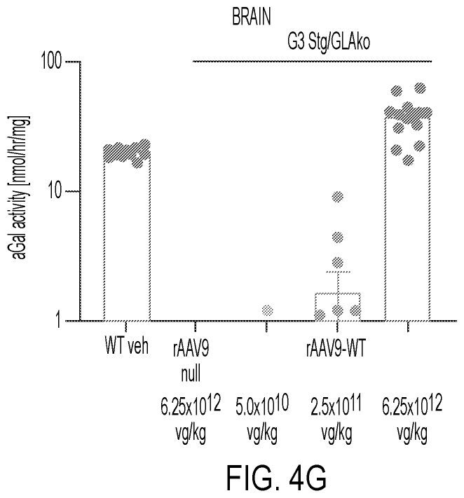

Treatment with rAAV9-WT increased both α-GAL levels and α-GAL activity in the serum in a sustained dose dependent manner (FIG. 4A). At the highest dose of 6.24e12 vg/kg, the serum α-GAL activity was 10,000-fold higher than WT serum α-GAL activity. Dose-dependent increases in tissue α-GAL levels and activity were observed in all tissues examined including the liver (FIG. 4B), kidney (FIG. 4C), heart (FIG. 4D), gastrointestinal tract (duodenum FIG. 4E and colon FIG. 4F) and the brain (FIG. 4G).

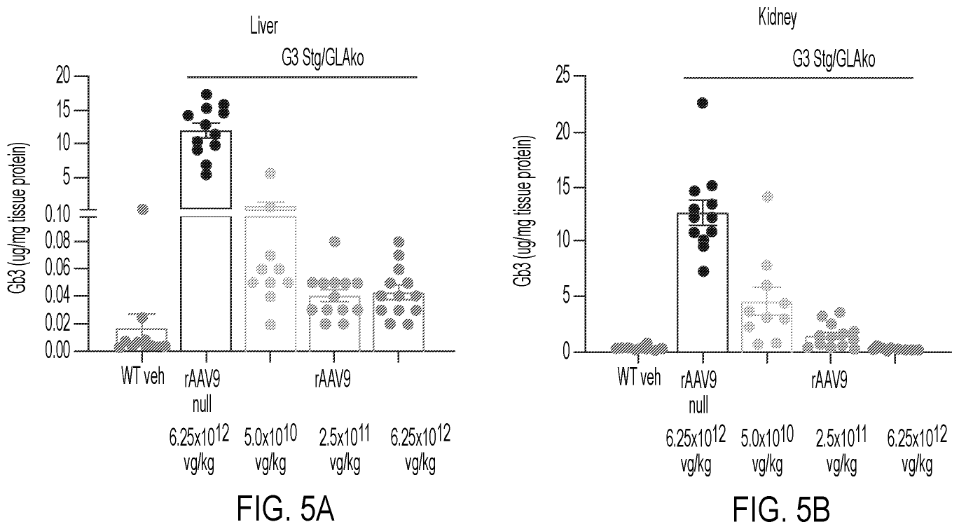

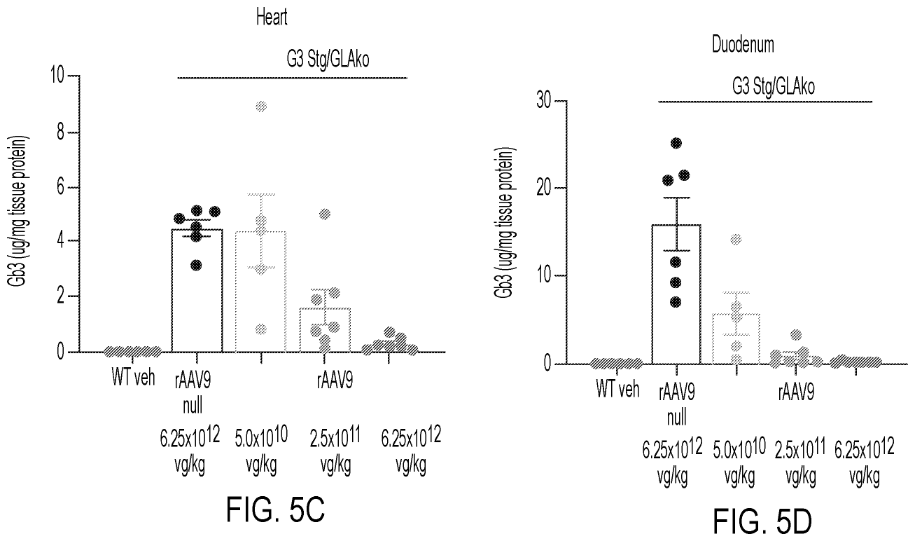

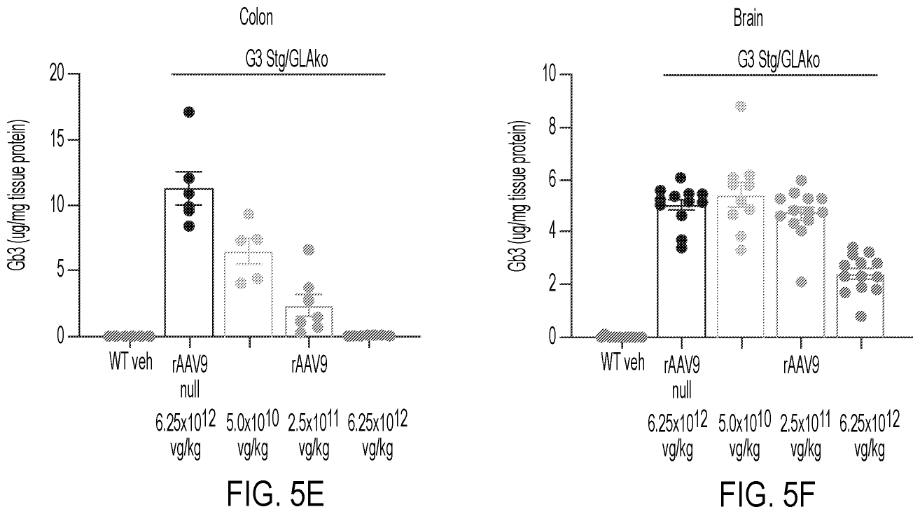

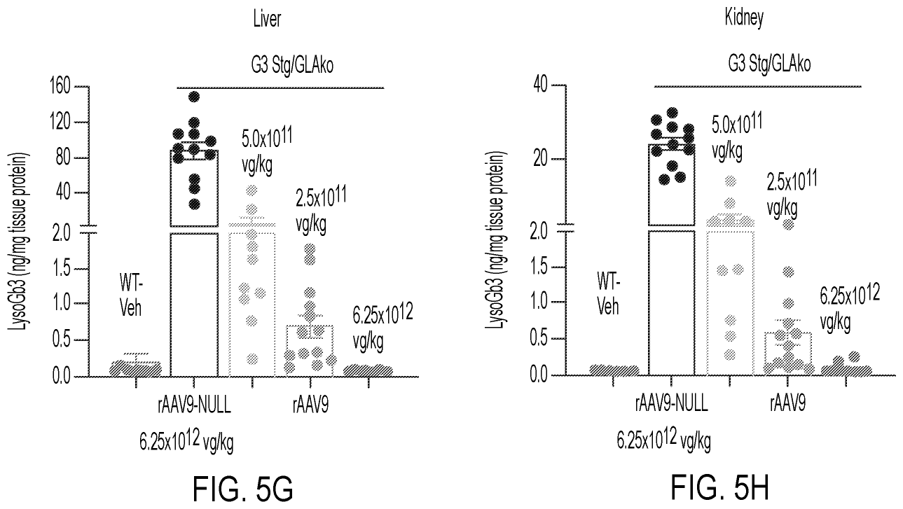

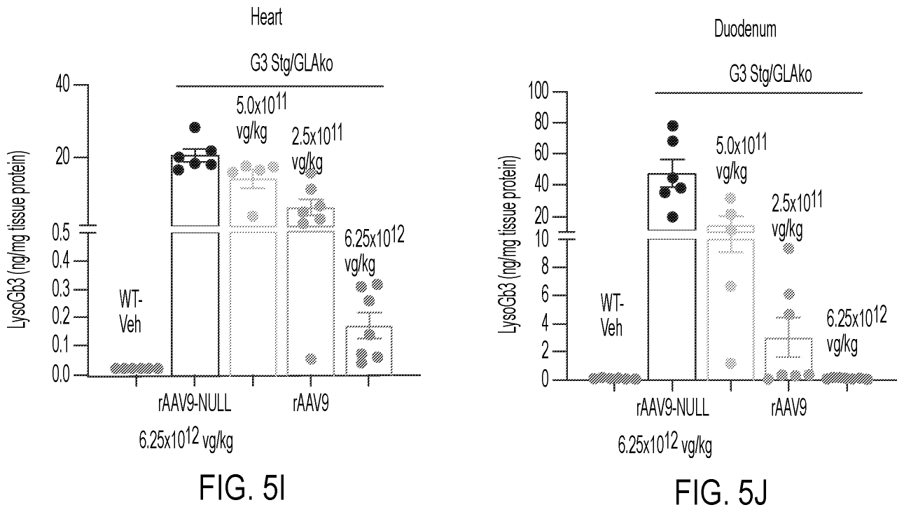

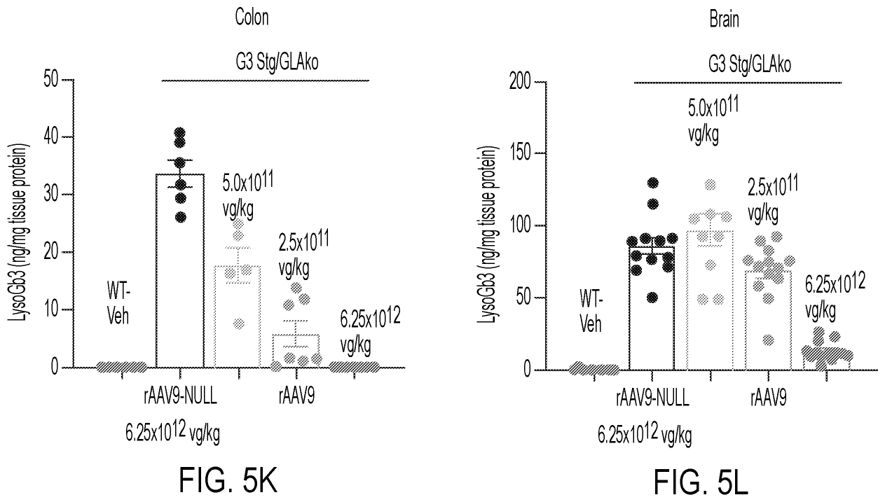

Next, the levels of GB3 in the severe Fabry (G3Stg/GLAko) mice transfected with rAAV9-WT encoding α-GAL were analyzed using mass-spectrometry. Briefly substrate samples were extracted first using Chloroform:Methanol (v/v 2:1) and formic acid before running in HPLC and LC-MS/MS (Applied Biosystem API5000, Turbo Ion Spray Ionization, positive-ion mode). It was observed that GB3 and lyso-Gb3 levels in various tissues from these animals showed a dose dependent decrease in the accumulated substrate levels in each tissue (FIG. 5A-5L).

It was observed that rAAV9-WT expressed α-GAL in Fabry mouse and reduced GB3 accumulation associated with Fabry.

### Example 4. Phenotypic Effects of rAAV9 on Fabry Mouse Model (G3Stg/GLAko Mice)

This Example shows the phenotypic effects of rAAV9-WT encoding α-GAL on G3Stg/GLAko mice. Fabry mice treated with rAAV9-WT showed overall improvement in body weight over time.

First, rAAV9-WT vector was produced and purified as described in Example 1 above.

Next, the G3Stg/GLAko mice, as described in Example 3 mice were treated with rAAV9 encoding α-GAL enzyme. The body weight was monitored through the duration of the study.

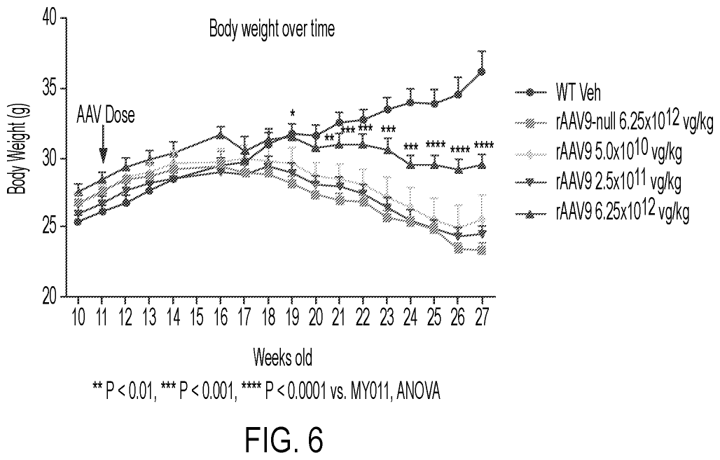

G3Stg/GLAko mice showed a marked decrease in body weight over the course of the study; mice treated with rAAV9-WT encoding α-GAL enzyme had higher body weights compared with mice given a null AAV vector (FIG. 6). WT mice showed a steady body weight gain throughout the study duration, however, G3Stg/GLAko mice treated with Null Vector as well as lower doses of rAAV9-WT started losing weight after 16-18 weeks of age such that the animals in the Null Vector group ended the study with lower body weights then the study start (22.2±0.5 in week 28 vs. 27.0±0.6 g/mouse at start, p<0.0001). Treatment with the highest dose of rAAV9-WT showed significantly lower weight loss at termination (28.5±0.8 g/mouse vs. 22.2±0.5 g/mouse in the null AAV, p<0.01). Treatment with high dose of rAAV9-WT thus prevented weight loss, demonstrating benefits of the gene therapy candidate to overall health.

The results of this Example demonstrated that rAAV9-WT encoding α-GAL enzyme can be used to cause significant improvement in treating symptoms of Fabry disease.

### Example 5. Restoration of Kidney Function Defects in Fabry Model (G3Stg/GLAko Mice) by rAAV9

This Example examined the effect of rAAV9-WT encoding α-GAL enzyme on kidney function in G3Stg/GLAko mice.

First, rAAV9-WT vector was produced and purified as described in Example 1 above.

Next, the G3Stg/GLAko mice, as described in Example 3 mice were treated with rAAV9. The kidney function was evaluated by measuring markers such as blood urea nitrogen, urine albumin levels.

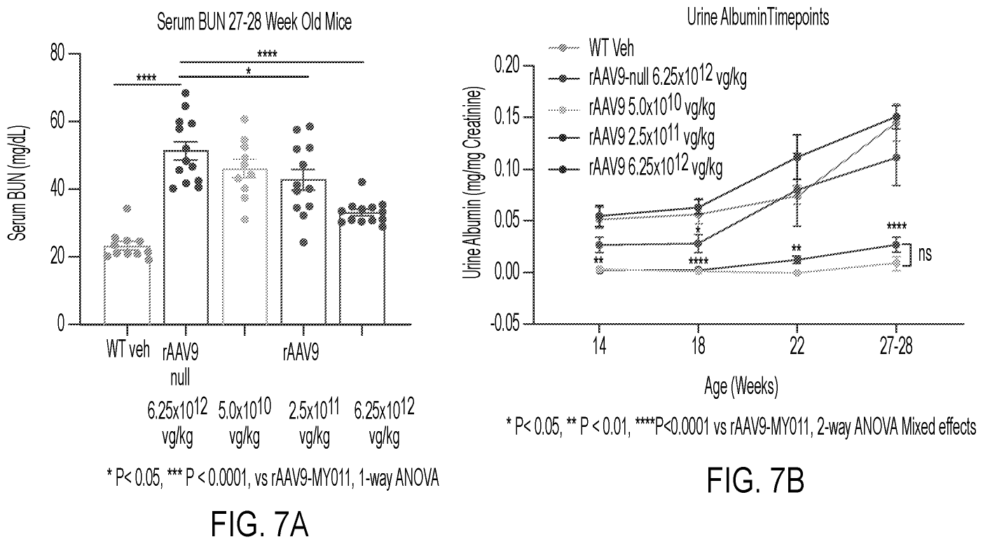

rAAV9-WT encoding α-GAL enzyme resulted in significant decreases in serum BUN in mice treated with the highest dose gene therapy (FIG. 7A). It was observed that the serum albumin levels in Fabry mice treated with the highest dose gene therapy (GT) were similar to that in WT mice (FIG. 7B).

This Example demonstrated that rAAV9-WT encoding α-GAL enzyme rescues renal phenotypes associated with Fabry disease. In particular, rAAV9-WT rescued BUN and urine albumin levels.

### Example 6. Restoration of Neuropathy Associated with Fabry Disease in Mouse Model (G3Stg/GLAko Mice)

This Example examined the effect of rAAV9-WT encoding α-GAL enzyme on the neuropathy markers in a Fabry mouse model.

First, rAAV9-WT vector was produced and purified as described in Example 1 above.

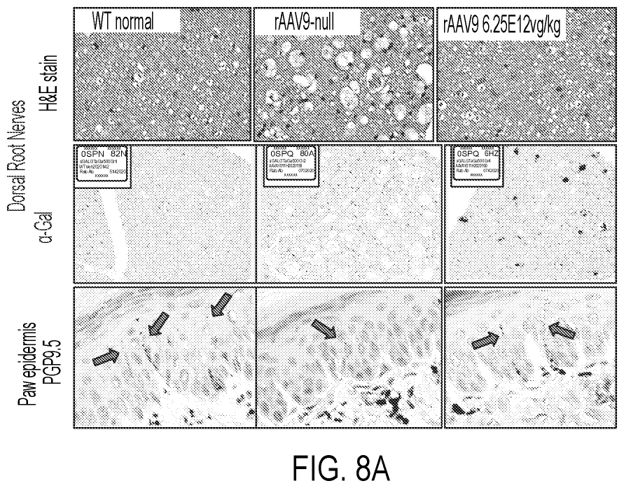

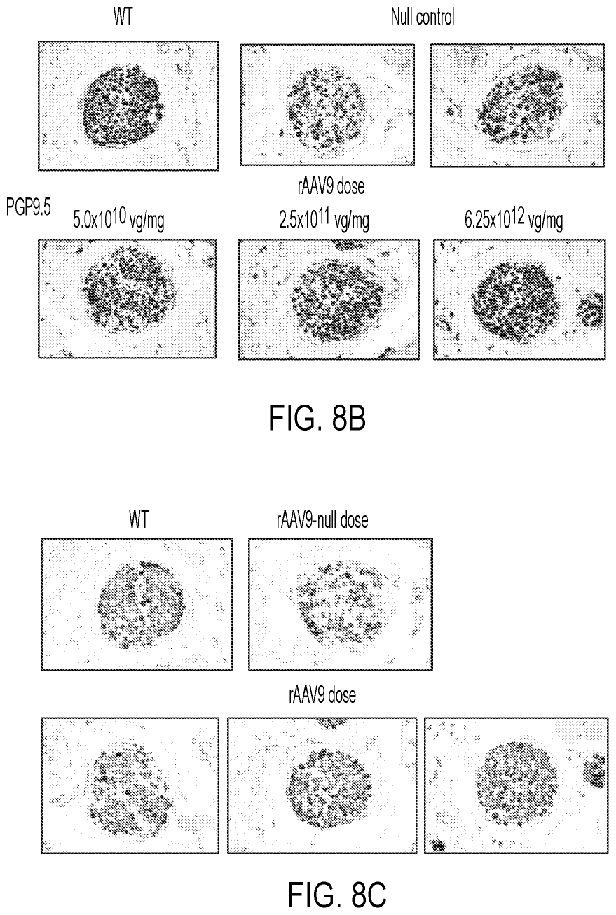

Next, the G3Stg/GLAko mice, as described in Example 3 mice were treated with rAAV9. G3Stg/GLAko mice exhibit several signs of neuropathy as was observed in the histology of peripheral neurons after sacrifice. Footpads from hind paws and dorsal root ganglion from these animals were collected for analyses by immunohistochemistry to evaluate small fiber neuron density to monitor any neuronal pathology in these animals. There was a notable reduction in vacuolation in dorsal root nerves, restored to WT levels, in animals treated with the highest dose GT (FIG. 8A). Finally, there was also a dose dependent increase in PGP9.5 (neuronal marker) and MPZ (marker for myelinated nerves) in the paws of treated animals (FIGS. 8B and 8C).

This Example demonstrated that rAAV9-WT produced α-GLA enzyme is associated with improvement of neuropathy as assessed by marker expression in a Fabry disease mouse model.

### Example 7. Normalized Autophagy Dysregulation in Fabry Disease Mouse Model (G3Stg/GLAko Mice)

This Example examined the effect of rAAV9-WT encoding α-GAL enzyme on the autophagy dysregulation in a Fabry mouse model.

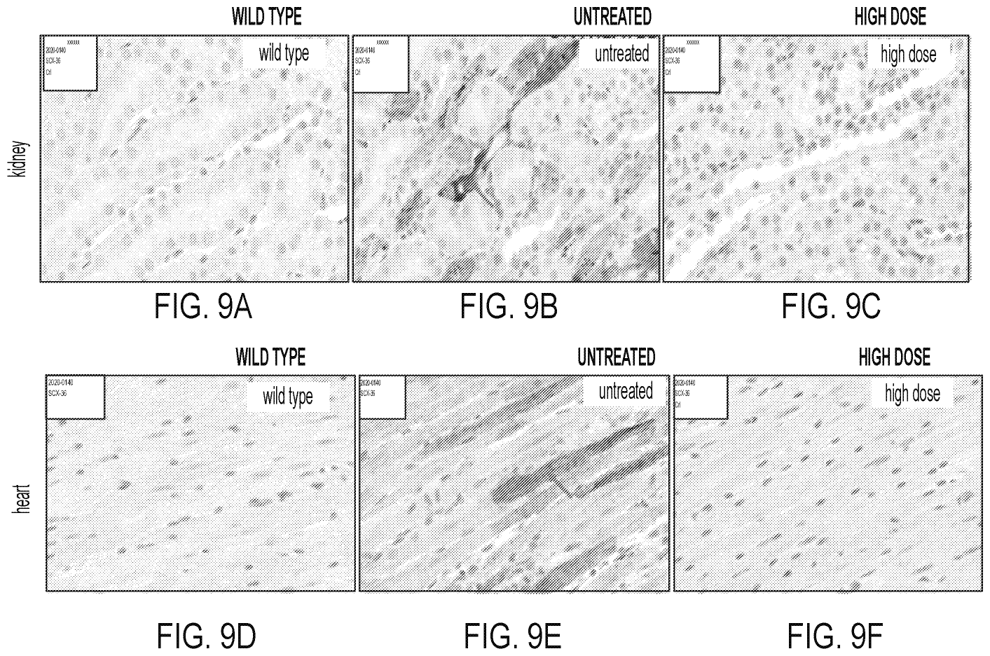

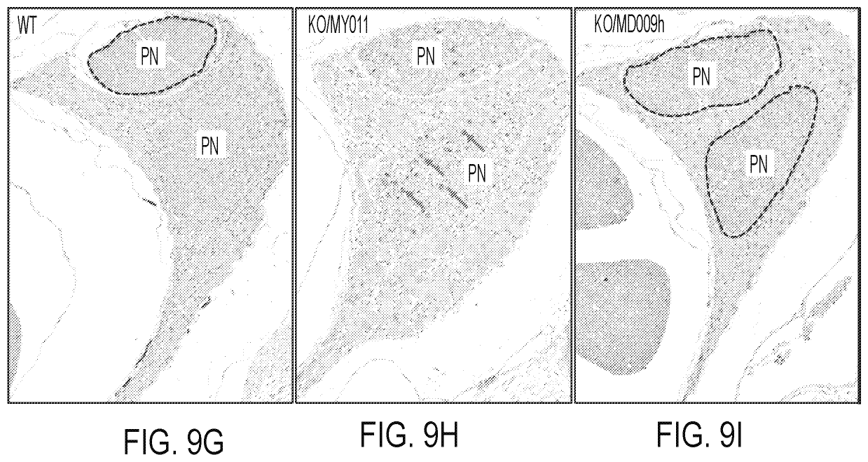

Autophagy dysregulation has been reported in Fabry patients (Chevrier et al, Autophagy. 2010 July; 6(5):589-99.) and may play a key role in Fabry neuropathy. Protein p62 is a classical receptor of autophagy, which builds up in Fabry patient kidneys and fibroblasts; similar accumulation was observed in the G3Stg/GLAko mice in the kidneys, heart and smooth muscles. Treatment with rAAV9-WT completely cleared p62 accumulation from the kidney and the heart when dosed at 6.25e12 vg/kg dose (FIG. 9A-FIG. 9F).

Additionally, chronic inflammation in Fabry patients contributes to organ damage (Pinto et al, High Blood Press Cardiovasc Prev. 2020) and is likely the cause of increase in DRG volume. Treatment of G3Stg/GLAko mice with rAAV9-WT resolves inflammation in the DRG as was demonstrated via reduction of the macrophage marker CD68 by histological assessment (FIG. 9G-FIG. 9I).

### Example 8. Ubiquitous Transduction Approach Using rAAV9 Administration Results in Higher Levels of Circulating α-GAL, Higher α-GAL Exposure in Target Tissues and Greater Substrate Reduction Compared to Liver Driven rAAV8 Approach

This Example shows that rAAV9-ubiquitous promoter-delivered GLA transgene provided higher α-GAL protein expression than rAAV8-liver specific promoter-delivered GLA in serum for at least 12 weeks.

First, rAAV9-WT and rAAV8-WT encoding α-GAL were produced and purified as described in Example 1 above. Next, 6-9 months old male GLAko mice were administered the purified rAAV9 or rAAV8 intravenously (IV) at, 2.5×10¹¹ vg/kg dose and the mice were followed for 12 weeks. Serum was collected at multiple time-points during the study as well as at the end of 12-weeks. In a subset of animals, 1 mg/kg dose of α-GAL ERT was administered 24 hours before sacrifice and collection of tissues to be used as positive control. Serum was collected 1 hour after administration of α-GAL ERT to capture the Cₘₐₓ levels of circulating enzyme.

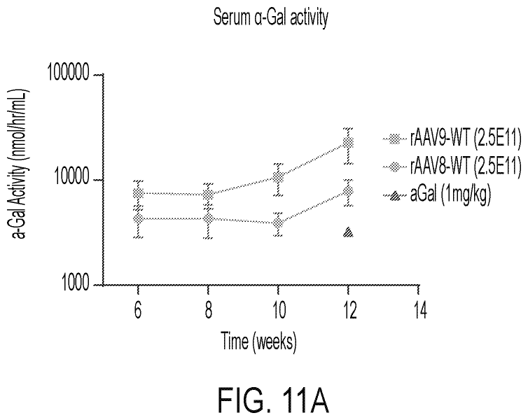

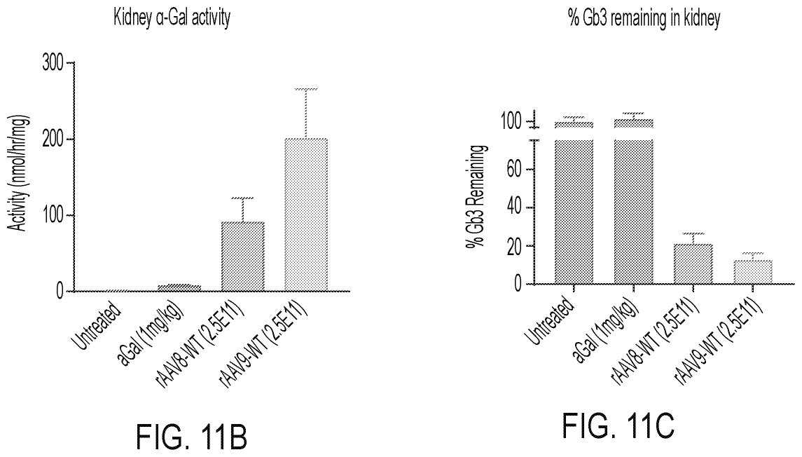

The quantity of α-GAL in the serum was measured using ELISA as described in Example 2. It was observed that rAAV9-WT resulted in higher steady level of serum α-GAL enzyme than rAAV8-WT and the Cₘₐₓ of the positive control α-GAL ERT (FIG. 11A).

At 12-weeks post administration, kidneys were harvested for further evaluation.

Significantly higher levels of α-GAL exposure was observed in the kidneys of animals treated with rAAV9-WT compared to rAAV8-WT or α-GAL (FIG. 11B), resulting in concomitant reduction in GB3 accumulation in these animals (FIG. 11C). It was further observed that treatment with rAAV9 reduced the GB3>86% while treatment with rAAV8 reduced GB3 by 78% while treatment with a single dose of α-GAL at 1 mg/kg did not result in any reduction in GB3 in the kidneys.

Therefore, overall it was observed that treatment with rAAV9 with a ubiquitous promoter driving GLA expression reduced GB3 in the kidneys more efficiently than a liver targeted rAAV8 and resulted in higher sustained α-GAL activity in serum for at least 12 weeks.

Without wishing to be bound by theory, it is contemplated that a liver specific promoter may also be used with a GLA transgene that has otherwise been modified/codon optimized, to express enzyme at increased levels.

### Example 9. Administration of rAAV9 with a Ubiquitous Promoter Driving GLA Expression Results in Dose Dependent Restoration of Kidney Function while a Liver Targeted rAAV8 Driving GLA Expression Improved Kidney Function Only at a High Dose

This Example examined the effect of rAAV9 and rAAV8 on kidney function in G3Stg/GLAko mice.

First, rAAV8 and rAAV9 were produced and purified as described in Example 1 above.

Next, the G3Stg/GLAko mice, as described in Example 3 mice were treated with rAAV8 and rAAV9. Wild type animals were treated with vehicle as control. Kidney function was evaluated by measuring blood urea nitrogen.

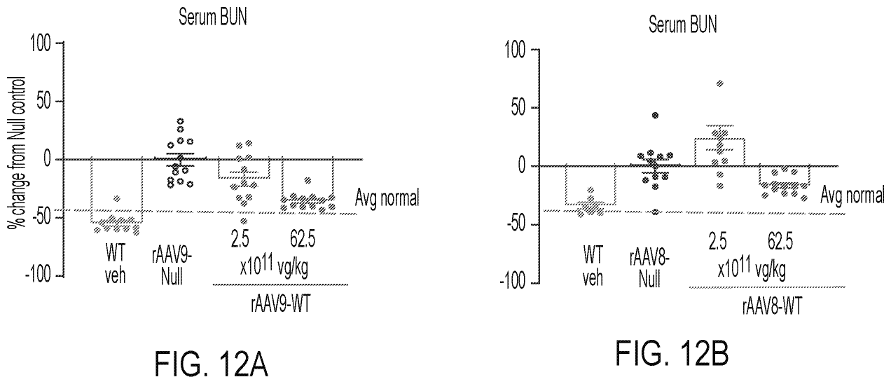

It was observed that rAAV9-WT resulted in dose dependent decreases in serum BUN, restoring levels close to that of healthy wild type animals at the highest dose of 6.25e12 vg/kg (FIG. 12A). Treatment of animals with the liver targeted rAAV8-WT resulted in no improvement of serum BUN at 2.5e11 vg/kg dose and only showed a response at the 25× higher dose of 6.25e12 vg/kg (FIG. 12B).

This Example demonstrated that rAAV9 driving GLA expression via a ubiquitous promoter rescues renal phenotypes associated with Fabry disease more efficiently than a liver targeted rAAV8.

### Example 10. Higher Exposure of α-GAL Variants Compared to Wildtype α-GAL in Various Tissues after Administration of Plasmids Expressing these Variants Via Hydrodynamic Tail Vein Injection

This Example shows serum stability and tissue biodistribution of the various α-GAL variants in vivo.

Plasmids expressing either wild type or engineered human alpha galactosidase (α-α-GAL) under a ubiquitous promoter were tested in a mouse model of Fabry disease. In the first study the following plasmids were tested: WT expresses wild type α-GAL while A, B, C, D, E, F express engineered α-GAL proteins. In the second study, the following plasmids were tested: WT expressing WT α-GAL protein while 002, 003, 004, 005, 006 and 007 expressing engineered α-GAL variants.

In the first study, 12-14-week-old male GLAko mice were administered with 50 ug plasmid DNA each via hydrodynamic gene delivery by tail vein injection. An arm was included in the study where GLAko mice were injected with buffer only as a negative control. Another arm was included in the study in which WT animals were treated with buffer. The animals were sacrificed 2 days post injection. Serum was collected by cardiac puncture at terminal endpoint and tissues such as the heart and kidney were collected after perfusion with PBS. Samples were snap frozen and stored at −80° C. Serum and tissue samples were analyzed for α-GAL activity.

In the second study, 10-12 week old male mice were used. The same study design as the first study was followed. An additional arm was included in this study in which the GLAko mice were injected with recombinant human α-GAL protein at 1 mg/kg dose as a positive control.

Tissues were homogenized in lysis buffer containing 10 mM HEPES with 0.5% Triton-X 100 and 1.5× Halt protease inhibitor cocktail, EDTA free, centrifuged and supernatant collected for analytical assays. Alpha galactosidase activity in supernatant or serum was measured using a fluorescent substrate. Briefly, 2 ul of biological samples were incubated with 15 uL 4-MU-α-GLA substrate solution (Research Products International Company, catalog #M65400) with α-galactosidase B inhibitor (N-acetyl-D-galactosamine, Sigma catalog #A-2795) at 37° C. for 60 minutes. The enzymatic reaction is stopped by addition of 200 uL glycine carbonate stop solution, pH 10.7. The 4-MU product was measured at the excitation wavelength 360 nm and emission wavelength 465 nm by a fluorescence plate reader. The concentrations of 4-MU in testing samples are calculated from the 4-MU calibration curve in the same plate. Tissue activity was normalized to total protein concentration determined by BCA assay.

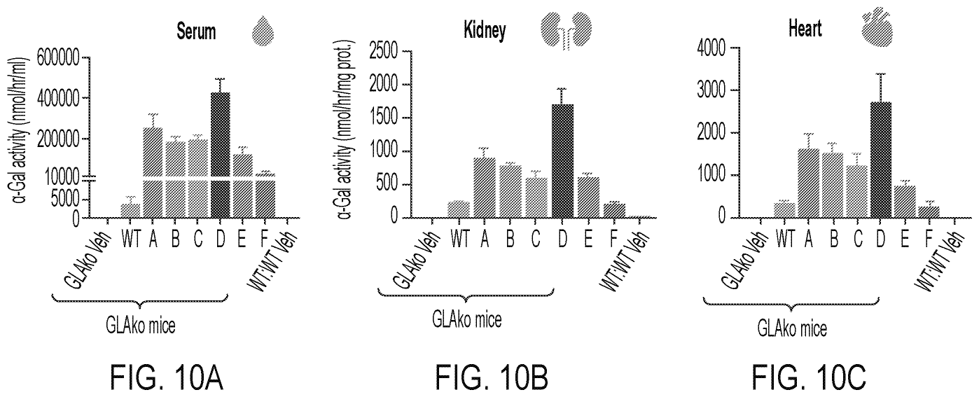

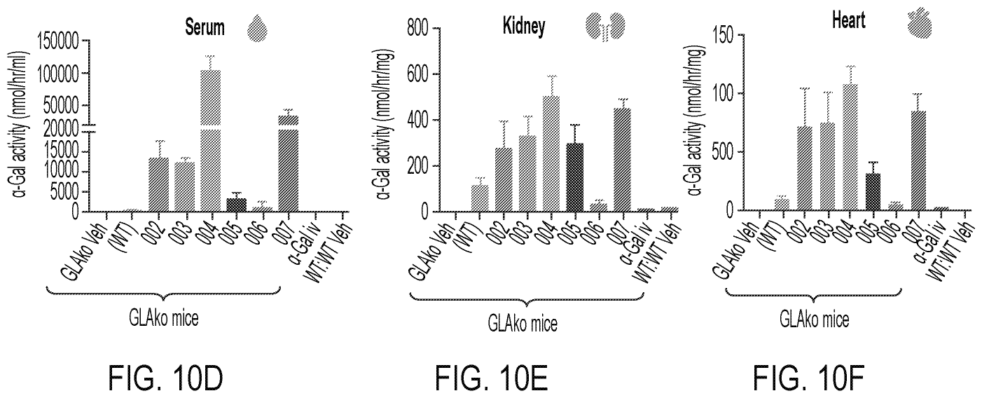

It was observed that in the first study, mice injected with plasmids A through F had significantly higher levels of α-GAL activity in circulation as well as in the heart and kidneys compared to that with WT (FIG. 10A-FIG. 10C). Plasmid A expresses wild type human α-GAL protein while the other plasmids expressed α-GAL variants that are engineered to improve serum stability and tissue uptake, which was reflected in these results. Plasmid D resulted in the highest α-GAL activity in both serum and tissues.

In the second study, mice injected with plasmids 002 through 007 (expressing engineered α-α-GAL) had significantly higher levels of α-GAL activity in circulation as well as in the heart and kidneys compared to that with 001 (expressing WT α-GAL) (FIG. 10D-FIG. 10F). In this study, the variant 004 resulted in the highest serum and tissue α-GAL activity.

This Example demonstrated that the plasmids containing various variant α-GAL transgenes express enzymes with significantly higher serum stability and tissue biodistribution compared to plasmids containing wild-type GLA.

### Example 11. Comparison of Vectors Expressing Engineered α-α-GAL and WT α-GAL

This Example examined enzyme activity in serum and tissues following administration of viral vectors encoding engineered α-α-GAL and WT α-α-GAL.

First, 4 variants of rAAV9 vector (described herein as rAAV9-WT, rAAV9-A, rAAV9-005, rAAV9-D were produced and purified as described in Example 1 above.

### Study 1

In study 1, 12-13 weeks old G3Stg/GLAko male mice were administered once with rAAV9-A, rAAV9-005, rAAV9-D, and rAAV9-WT at 2 different doses 5.0×10¹⁰ and 2.5×10¹¹ vg/kg, or with a null control at 2.5×10¹¹ vg/kg and monitored for 4 weeks post dose. Table 3 shows the particular α-GAL variant used in each rAAV9. WT:WT sibling mice with the same genetic background were used as control and were administered with vehicle only. Mice were assigned to each test article group in a semi-randomized process based on pre-dose body weights to ensure balanced groups. Serum was collected during the study at multiple time points. Blood was collected via retro-orbital or tail vein bleed during the study and via cardiac puncture at termination and processed to collect serum. At the end of the study, terminal serum was collected, and mice were perfused for collection of organs, including liver, kidney and heart then snap frozen in dry ice and stored at −80° C. Analytical evaluations included measurement of α-GAL enzyme activity, and analyses of substrate levels in serum and various tissues.

TABLE 3
rAAV9 - α-GAL variant vector summary

| | α- | Protein | Nucleic Acid |
|---|---|---|---|
| rAAV9 vector | α-GAL | comprises | comprises |
| name | variant | SEQ ID NO | SEQ ID NO |
| rAAV9-WT | WILD TYPE | SEQ ID NO: 30 | SEQ ID NO: 3 |
| rAAV9-A | A | SEQ ID NO: 7 | SEQ ID NO: 18 |
| rAAV9-005 | 005 | SEQ ID NO: 15 | SEQ ID NO: 27 |
| rAAV9-D | D | SEQ ID NO: 10 | SEQ ID NO: 37 |
| rAAV9-40 | 004 | SEQ ID NO: 14 | SEQ ID NO: 26 |
| rAAV9-41 | 008 | SEQ ID NO: 33 | SEQ ID NO: 31 |
| rAAV9-42 | 009 | SEQ ID NO: 34 | SEQ ID NO: 32 |

### Alpha Galactosidase Activity:

Tissues were homogenized in lysis buffer containing 10 mM HEPES with 0.5% Triton-X 100 and 1.5× Halt protease inhibitor cocktail, EDTA free, centrifuged and supernatant collected for analytical assays. Alpha galactosidase activity in supernatant or serum was measured using a fluorescent substrate. Briefly, 2 ul of biological samples were incubated with 15 uL 4-MU-a-gal substrate solution (Research Products International Company, catalog #M65400) with a-galactosidase B inhibitor (N-acetyl-D-galactosamine, Sigma catalog #A-2795) at 37° C. for 60 minutes. The enzymatic reaction is stopped by addition of 200 uL glycine carbonate stop solution, pH 10.7. The 4-MU product is measured at the excitation wavelength 360 nm and emission wavelength 465 nm by a fluorescence plate reader. The concentrations of 4-MU in testing samples are calculated from the 4-MU calibration curve in the same plate. Tissue activity is normalized to total protein concentration determined by BCA assay (Thermo Scientific, catalog #23225).

Mus musculus, G3Stg/GLA knockout mice were generated by crossing GLA knockout mice, which have a C57BL/6 background, with GB3 synthase transgenic mice. The founders were purchased from Jackson labs and the breeding colony was maintained at Taconic Biosciences. Animals were maintained in a controlled environment on a 12 h dark/12 h light cycle (lights on at 7:00 am) with no more than 4 mice per cage in a ventilated cage rack system at Takeda or Melior Discoveries and fed standard rodent chow and water ad libitum.

Single intravenous administration of rAAV9-A, rAAV9-005 and rAAV9-D at either 5.0×10¹⁰ vg/kg or 2.5×10¹¹ vg/kg to male G3Stg/GLAko mice resulted in sustained higher α-GAL activity in serum compared to administration of rAAV9-WT over the 4-week duration of the study.

### Study 2

In study 2, 14-16 weeks old G3Stg/GLAko male mice were administered once with rAAV9-40, rAAV9-41, rAAV9-42, rAAV9-WT, or with null vector at 2.5×10¹¹ vg/kg dose and monitored for 4 weeks post dose. Table 3 shows the particular α-GAL variants used in each rAAV9. WT:WT sibling mice were used as control and administered with vehicle only. The same study design as the previous study was followed. in which older G3Stg/GLAko mice (14-16 weeks old) were used, a single dose at 2.5×10¹¹ vg/kg of rAAV9-40, rAAV9-41 and rAAV9-42 also produced sustained α-GAL activity in serum throughout 4 weeks and at a higher level than rAAV9-WT treated animals.

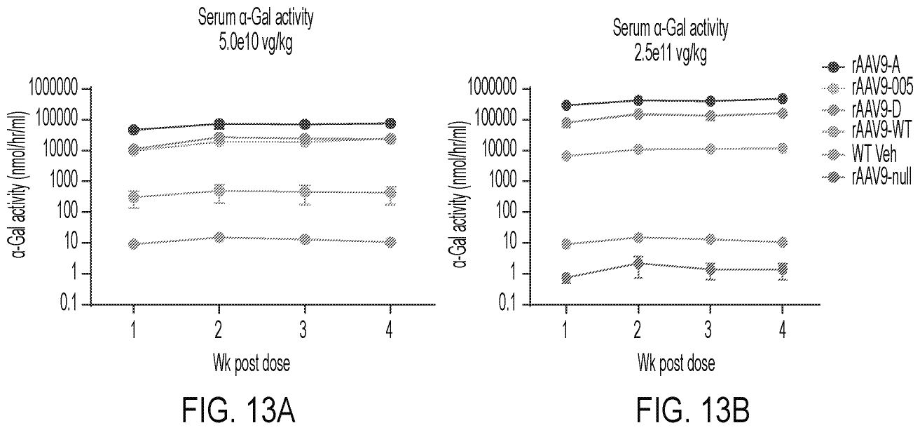

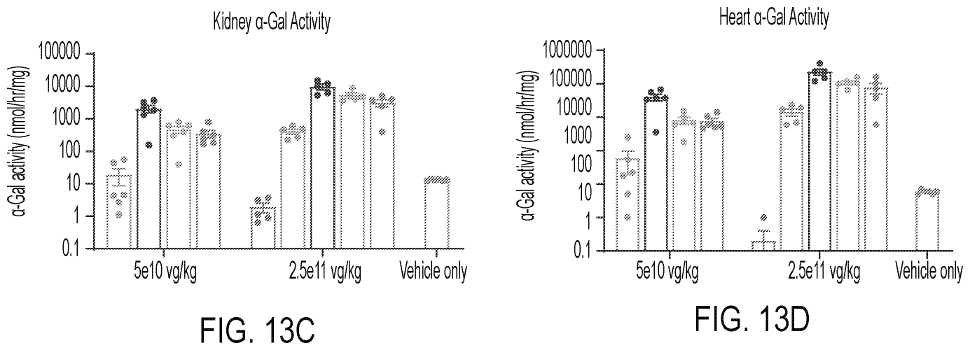

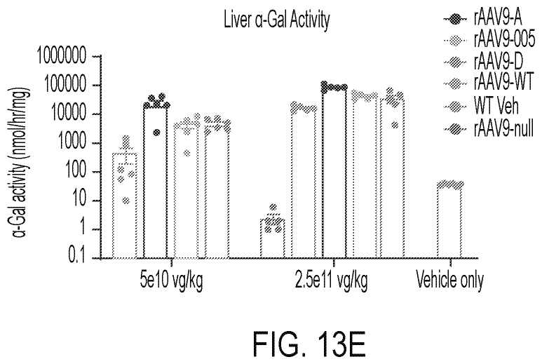

FIG. 13A-FIG. 13B depict α-galactosidase activity of each variant in serum. It was observed that the α-GAL activity in serum of animals dosed with rAAV9-A, rAAV9-005 and rAAV9-D at 2.5×10¹¹ vg/kg dose was more than 16,000 fold, 16,000 fold and 46,000 fold respectively above normal α-GAL activity in WT mice, as measured in the WT:WT animals treated with vehicle. In contrast, α-GAL serum activity after rAAV9-WT administration reached just above 1,000 fold over normal. Further, elevated α-GAL activity in tissues (kidney, heart and liver) was also observed in rAAV9-A, rAAV9-005 and rAAV9-D treated animals, compared to ones treated with rAAV9-WT group at 2 different doses. FIG. 13C-FIG. 13E summarize the α-galactosidase activity of each variant in kidney, heart and liver.

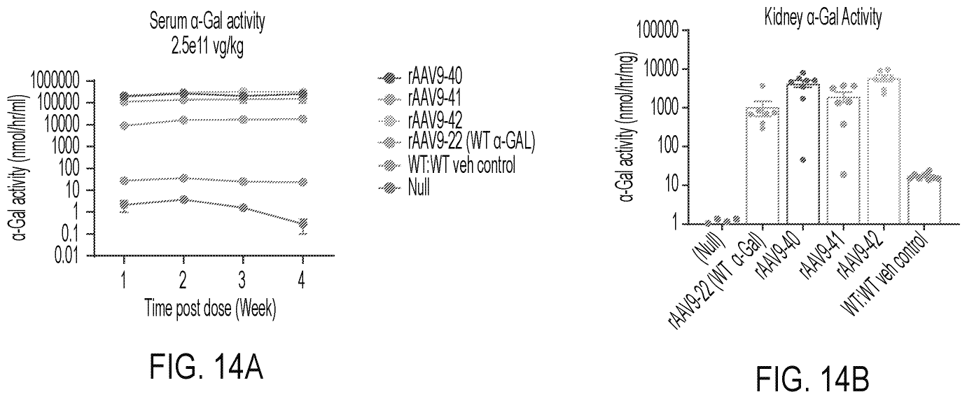

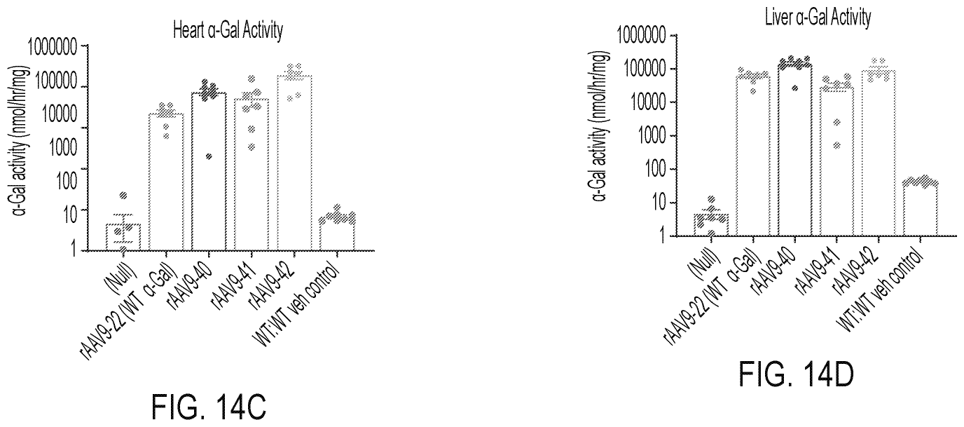

FIG. 14A depicts serum α-galactosidase activity of various variants in G3Stg/GLAko mice (14-16 weeks old). Elevated serum α-GAL activity in rAAV9-40 and rAAV9-42 group was above 11,000 fold compared to WT:WT vehicle control, and above 6,600 fold in rAAV9-41 group, while serum α-GAL activity in rAAV9-WT treated animals was only 780 fold above WT:WT control group. All tissues evaluated in rAAV9-40, rAAV9-42 and rAAV9-41 group also demonstrated higher α-GAL activity than rAAV9-WT treated animals, except in the liver of rAAV9-41 animals, which was lower than that of animals treated with rAAV9-WT (FIG. 14B-FIG. 14D).

Table 4 summarizes the serum and tissue α-α-GAL activities of each variant.

TABLE 4
Serum and tissue α-GAL activity at 4 weeks post IV administration of variants compared to null control
α-GAL activity at a dose of 2.5 × 10¹¹ vg/kg

| | WT:WT Veh | | | rAAV9-WT | | | rAAV9-A | | | rAAV9-005 | | | rAAV9-D | | |
|---|---|---|---|---|---|---|---|---|---|---|---|---|---|---|---|
| | Act. | | | Act. | | | Act. | | | Act. | | | Act. | | |
| | nmol/hr/mL | | | nmol/hr/mL | | | nmol/hr/mL | | | nmol/hr/mL | | | nmol/hr/mL | | |
| | Serum | | | Serum | | | Serum | | | Serum | | | Serum | | |
| Serum, | or mg of | | | or mg of | | | or mg of | | | or mg of | | | or mg of | | |
| Tissue | Prot | SEM | N | Prot | SEM | N | Prot | SEM | N | Prot | SEM | N | Prot | SEM | N |
| Serum | 11 | 2 | 6 | 423.9 | 247 | 6 | 76503 | 18281 | 6 | 24553 | 6758 | 6 | 23242 | 4973 | 6 |
| Kidney | 13 | 0 | 6 | 19 | 10 | 6 | 2052 | 520 | 6 | 468 | 112 | 6 | 350 | 94 | 6 |
| Heart | 6 | 0 | 6 | 58 | 39 | 6 | 3951 | 892 | 6 | 799 | 188 | 6 | 758 | 158 | 6 |
| Liver | 37 | 2 | 6 | 432 | 241 | 6 | 23923 | 5646 | 6 | 4367 | 1145 | 6 | 4623 | 812 | 6 |

α-GAL activity at a dose of 5.0 × 10¹⁰ vg/kg

| | WT:WT Veh | | | rAAV9-null | | | rAAV9-WT | | | rAAV9-A | | | rAAV9-005 | | | rAAV9-D | | |
|---|---|---|---|---|---|---|---|---|---|---|---|---|---|---|---|---|---|---|
| | Act. | | | Act. | | | Act. | | | Act. | | | Act. | | | Act. | | |
| | nmol/hr/mL | | | nmol/hr/mL | | | nmol/hr/mL | | | nmol/hr/mL | | | nmol/hr/mL | | | nmol/hr/mL | | |
| | Serum | | | Serum | | | Serum | | | Serum | | | Serum | | | Serum | | |
| Serum, | or mg of | | | or mg of | | | or mg of | | | or mg of | | | or mg of | | | or mg of | | |
| Tissue | Prot | SEM | N | Prot | SEM | N | Prot | SEM | N | Prot | SEM | N | Prot | SEM | N | Prot | SEM | N |
| Serum | 11 | 2 | 6 | 1 | 1 | 5 | 11757 | 2914 | 5 | 484606 | 46941 | 5 | 162977 | 15844 | 5 | 160410 | 35695 | 5 |
| Kidney | 13 | 0 | 6 | 2 | 1 | 5 | 410 | 71 | 5 | 9824 | 1822 | 5 | 5321 | 962 | 5 | 3100 | 788 | 5 |
| Heart | 6 | 0 | 6 | 0 | 0 | 5 | 1439 | 343 | 5 | 22833 | 5065 | 5 | 10866 | 1499 | 5 | 7653 | 2700 | 5 |
| Liver | 37 | 2 | 6 | 3 | 1 | 5 | 16734 | 1687 | 5 | 86567 | 7160 | 5 | 43162 | 4175 | 5 | 34543 | 10554 | 5 |

α-GAL activity at a dose of 5.0 × 10¹⁰ vg/kg at 2.5 × 10¹¹ vg/kg from Study 2

| | WT:WT Veh | | | rAAV9-null | | | rAAV9-WT | | | rAAV9-A | | | rAAV9-005 | | | rAAV9-D | | |
|---|---|---|---|---|---|---|---|---|---|---|---|---|---|---|---|---|---|---|
| | Act. | | | Act. | | | Act. | | | Act. | | | Act. | | | Act. | | |
| | nmol/hr/mL | | | nmol/hr/mL | | | nmol/hr/mL | | | nmol/hr/mL | | | nmol/hr/mL | | | nmol/hr/mL | | |
| | Serum | | | Serum | | | Serum | | | Serum | | | Serum | | | Serum | | |
| Serum, | or mg of | | | or mg of | | | or mg of | | | or mg of | | | or mg of | | | or mg of | | |
| Tissue | Prot | SEM | N | Prot | SEM | N | Prot | SEM | N | Prot | SEM | N | Prot | SEM | N | Prot | SEM | N |
| Serum | 23 | 1 | 8 | 0 | 0 | 7 | 17938 | 2629 | 7 | 253154 | 60153 | 8 | 151451 | 55459 | 7 | 309866 | 75811 | 7 |
| Kidney | 1 | 0 | 8 | 0 | 0 | 7 | 613 | 87 | 7 | 4263 | 880 | 8 | 1968 | 589 | 7 | 5936 | 1052 | 7 |
| Heart | 7 | 1 | 8 | 5 | 3 | 7 | 2270 | 423 | 7 | 7445 | 1392 | 8 | 5172 | 1964 | 7 | 19168 | 4040 | 7 |
| Liver | 42 | 2 | 8 | 5 | 1 | 7 | 60999 | 8904 | 7 | 142489 | 21338 | 8 | 29357 | 8232 | 7 | 93335 | 21958 | 7 |
| Serum | 23 | 1 | 8 | 0 | 0 | 7 | 17938 | 2629 | 7 | 253154 | 60153 | 8 | 151451 | 55459 | 7 | 309866 | 75811 | 7 |

### Example 12. Comparison of GB3 and lysoGb3 Substrates Reduction in Variants

This Example examined the effects of engineered α-GAL and WT α-GAL variants on GB3 and lysoGb3 reduction.

GB3 and lysoGb3 Substrate Quantification

Substrates from serum and tissue samples were analyzed using an LC-MS method. Samples were extracted first using Chloroform:Methanol (v/v 2:1) and formic acid before running in HPLC and LC-MS/MS (Applied Biosystem API5000, Turbo Ion Spray Ionization, positive-ion mode).

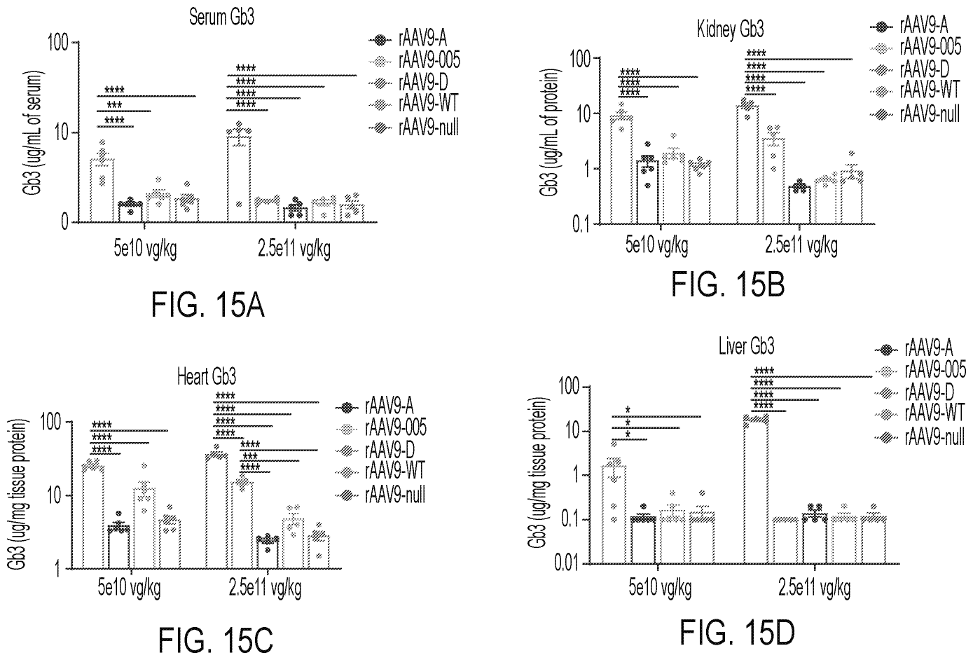

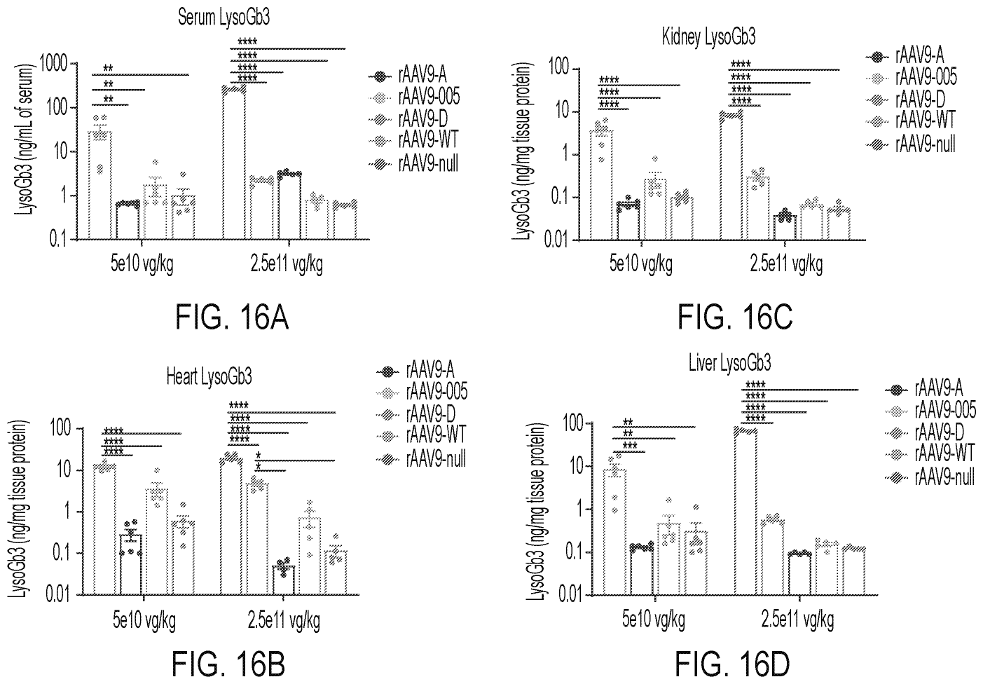

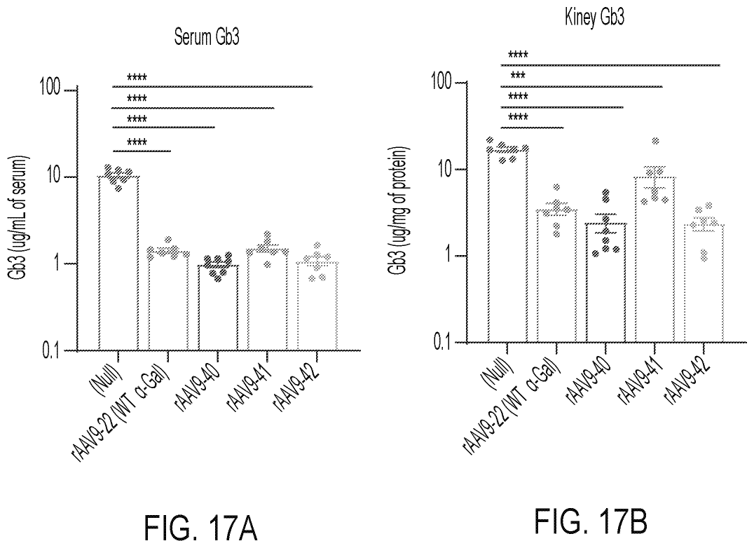

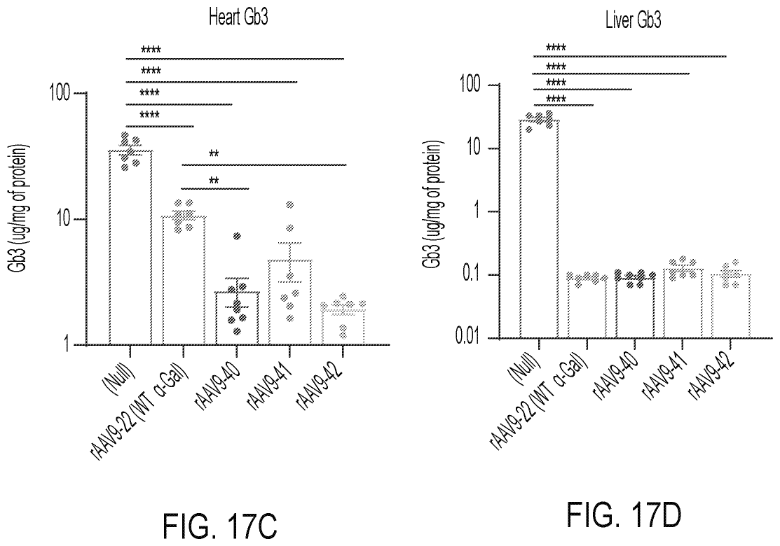

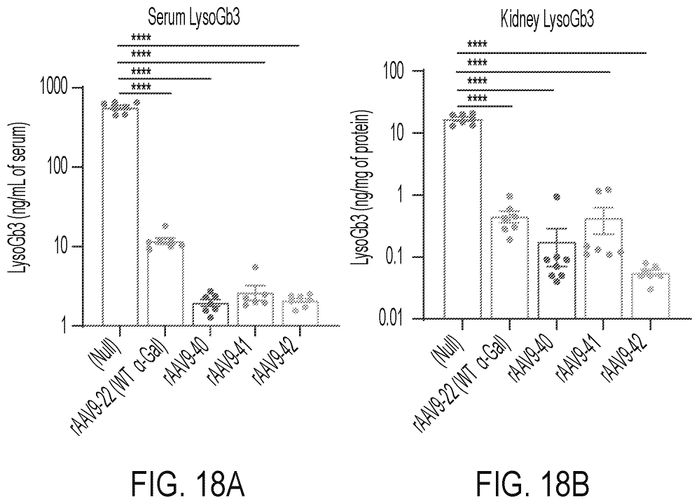

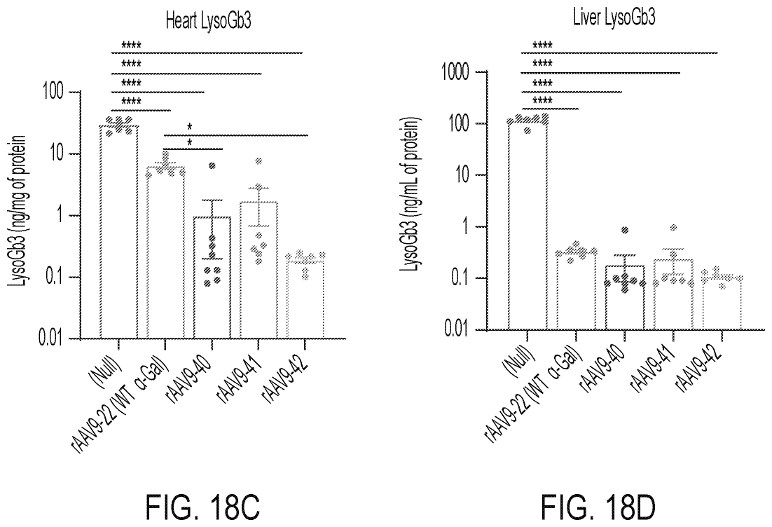

It was observed that with sustained high α-GAL activity in serum and tissues, both GB3 and lysoGb3 substrates were reduced substantially in mice treated with viral vectors expressing α-GAL compared to rAAV9-Null control in both studies. FIG. 15A-15D summarizes serum and tissue GB3 at 4 weeks post IV administration of rAAV9-A, rAAV9-005, rAAV9-D, rAAV9-WT, Null control at 2 different doses from Study 1 (described in Example 10). FIG. 16A-16D summarizes serum and tissue lysoGb3 at 4 weeks post IV administration of rAAV9-A, rAAV9-005, rAAV9-D, rAAV9-WT, Null control at 2 different doses from Study 1. FIG. 17A-17D summarizes serum and tissue GB3 at 4 weeks post IV administration of rAAV9-A, rAAV9-005, rAAV9-D, rAAV9-WT, Null control at 2 different doses from Study 2 (described in Example 10). FIG. 18A-18D summarizes serum and tissue lysoGb3 at 4 weeks post IV administration of rAAV9-A, rAAV9-005, rAAV9-D, rAAV9-WT, Null control at 2 different doses from Study 2. In the liver, where α-GAL activity was the highest after dosing of viral vectors, all viral vectors were equally efficient in reducing substrate close to zero after being dosed at 2.5×10¹¹ vg/kg dose. In the heart and kidneys, rAAV9-A, rAAV9-005, rAAV9-D, rAAV9-41 and rAAV9-42 were more efficient than rAAV9-WT, in reducing GB3. One exception to this was the kidneys in rAAV9-41 treated animals. Table 5 shows serum and tissue GB3 and lysoGb3 substrates at 4 weeks post IV administration of rAAV9-A, rAAV9-005, rAAV9-D, rAAV9-WT, rAAV9 Null control at 2.5×10¹¹ vg/kg and 5.0×10¹⁰ vg/kg doses from Study 1. Table 6 shows Serum and tissue GB3 and lysoGb3 substrates at 4 weeks post IV administration of rAAV9-40, rAAV9-41, rAAV9-42, rAAV9-WT, rAAV9-Null control at 2.5×10¹¹ vg/kg from study 2.

Table 7 shows comparison of tissue GB3 substrate reduction in percentage of G3Stg/GLAko Null control group (rAAV9-null) at 4 weeks post administration of rAAV9 test articles from study 1 and study 2.

GB3 reduction in the heart was at or greater than 86% in animals treated with viral vectors expressing engineered α-GAL (as high as 95% in rAAV9-42 treated animals) compared to animals treated with null vector. In contrast, GB3 was reduced in the heart by only 70% and 58% in animals treated with rAAV9-WT in studies 1 and 2 respectively (Table 7). Substrate accumulation occurs progressively with age in this mouse model. Since older mice were used in study 2, the percentage of substrate clearance by rAAV9-WT was lower in this study than in study 1. Kidney GB3 reduction post treatment with rAAV9-A, rAAV9-005 and rAAV9-D was 96%, 95% and 93% respectively, compared to 74% reduction in kidney GB3 in animals treated with rAAV9-WT in study 1 (Table 7). In study 2, treatment with rAAV9-40, rAAV9-41, and rAAV9-42 reduced kidney GB3 substrates by 86%, 51% and 86% respectively compared to null vector treated animals, while the reduction in animals treated with rAAV9-WT was 79% (Table 7). At 2.5e11 vg/kg dose of viral vectors expressing engineered α-GAL variants in Fabry symptomatic mice, lysoGb3 substrate levels were nearly normalized to that of WT:WT mice treated with vehicle (Table 5 and Table 6).

TABLE 5
Serum and tissueGB3 and lysoGb3 substrates at 4 weeks post IV administration From Study 1.

| | WT:WT Veh | | | rAAV9-null control | | | rAAV9-22 | | | rAAV9-A | | | rAAV9-005 | | | rAAV9-D | | |
|---|---|---|---|---|---|---|---|---|---|---|---|---|---|---|---|---|---|---|
| | Gb3 | | | Gb3 | | | Gb3 | | | Gb3 | | | Gb3 | | | Gb3 | | |
| | ug/mL | | | ug/mL | | | ug/mL | | | ug/mL | | | ug/mL | | | ug/mL | | |
| Serum, | Serum or mg | | | Serum or mg | | | Serum or mg | | | Serum or mg | | | Serum or mg | | | Serum or mg | | |
| Tissue | of Prot | SEM | N | of Prot | SEM | N | of Prot | SEM | N | of Prot | SEM | N | of Prot | SEM | N | of Prot | SEM | N |
| Serum | 0.10 | 0.00 | 6 | 9.06 | 1.91 | 5 | 1.74 | 0.05 | 5 | 1.46 | 0.12 | 5 | 1.66 | 0.12 | 5 | 1.58 | 0.16 | 5 |
| Kidney | 0.88 | 0.11 | 6 | 13.82 | 1.57 | 5 | 3.58 | 0.95 | 5 | 0.48 | 0.04 | 5 | 0.64 | 0.05 | 5 | 0.92 | 0.25 | 5 |
| Heart | 0.00 | 0.00 | 6 | 36.38 | 2.59 | 5 | 15.20 | 1.10 | 5 | 2.42 | 0.17 | 5 | 4.84 | 0.84 | 5 | 2.82 | 0.40 | 5 |
| Liver | 0.00 | 0.00 | 6 | 18.46 | 1.47 | 5 | 0.10 | 0.00 | 5 | 0.14 | 0.02 | 5 | 0.12 | 0.02 | 5 | 0.12 | 0.02 | 5 |
| | LysoGb3 | | | LysoGb3 | | | LysoGb3 | | | LysoGb3 | | | LysoGb3 | | | LysoGb3 | | |
| | ng/mL | | | ng/mL | | | ng/mL | | | ng/mL | | | ng/mL | | | ng/mL | | |
| Serum, | Serum or mg | | | Serum or mg | | | Serum or mg | | | Serum or mg | | | Serum or mg | | | Serum or mg | | |
| Tissue | of Prot | SEM | N | of Prot | SEM | N | of Prot | SEM | N | of Prot | SEM | N | of Prot | SEM | N | of Prot | SEM | N |
| Serum | 2.89 | 0.12 | 6 | 261.80 | 15.36 | 5 | 2.21 | 0.17 | 5 | 3.12 | 0.17 | 5 | 0.79 | 0.10 | 5 | 0.61 | 0.04 | 5 |
| Kidney | 0.04 | 0.00 | 6 | 8.42 | 0.68 | 5 | 0.30 | 0.05 | 5 | 0.04 | 0.00 | 5 | 0.07 | 0.01 | 5 | 0.05 | 0.01 | 5 |
| Heart | 0.00 | 0.00 | 6 | 19.49 | 2.05 | 5 | 4.80 | 0.63 | 5 | 0.05 | 0.01 | 5 | 0.71 | 0.30 | 5 | 0.12 | 0.04 | 5 |
| Liver | 0.08 | 0.00 | 6 | 66.27 | 3.00 | 5 | 0.57 | 0.05 | 5 | 0.09 | 0.00 | 5 | 0.15 | 0.02 | 5 | 0.12 | 0.00 | 5 |

| | WT:WT Veh | | | rAAV9-WT | | | rAAV9-A | | | rAAV9-005 | | | rAAV9-D | | |
|---|---|---|---|---|---|---|---|---|---|---|---|---|---|---|---|
| | Gb3 | | | Gb3 | | | Gb3 | | | Gb3 | | | Gb3 | | |
| | ug/mL | | | ug/mL | | | ug/mL | | | ug/mL | | | ug/mL | | |
| Serum, | Serum or mg | | | Serum or mg | | | Serum or mg | | | Serum or mg | | | Serum or mg | | |
| Tissue | of Prot | SEM | N | of Prot | SEM | N | of Prot | SEM | N | of Prot | SEM | N | of Prot | SEM | N |
| Serum | 0.10 | 0.00 | 6 | 5.08 | 0.79 | 5 | 1.58 | 0.07 | 5 | 2.08 | 0.20 | 5 | 1.85 | 0.18 | 5 |
| Kidney | 0.88 | 0.11 | 6 | 9.17 | 1.35 | 5 | 1.40 | 0.33 | 5 | 1.93 | 0.42 | 5 | 1.17 | 0.10 | 5 |
| Heart | 0.00 | 0.00 | 6 | 25.42 | 1.43 | 5 | 3.90 | 0.39 | 5 | 12.50 | 2.82 | 5 | 4.67 | 0.55 | 5 |
| Liver | 0.00 | 0.00 | 6 | 1.67 | 0.76 | 5 | 0.12 | 0.02 | 5 | 0.17 | 0.05 | 5 | 0.15 | 0.05 | 5 |
| | LysoGb3 | | | LysoGb3 | | | LysoGb3 | | | LysoGb3 | | | LysoGb3 | | |
| | ng/mL | | | ng/mL | | | ng/mL | | | ng/mL | | | ng/mL | | |
| Serum, | Serum or mg | | | Serum or mg | | | Serum or mg | | | Serum or mg | | | Serum or mg | | |
| Tissue | of Prot | SEM | N | of Prot | SEM | N | of Prot | SEM | N | of Prot | SEM | N | of Prot | SEM | N |
| Serum | 2.89 | 0.12 | 6 | 29.13 | 10.39 | 5 | 0.66 | 0.02 | 5 | 1.76 | 0.83 | 5 | 1.01 | 0.41 | 5 |
| Kidney | 0.04 | 0.00 | 6 | 3.70 | 0.88 | 5 | 0.07 | 0.01 | 5 | 0.27 | 0.11 | 5 | 0.10 | 0.01 | 5 |
| Heart | 0.00 | 0.00 | 6 | 12.25 | 0.95 | 5 | 0.29 | 0.09 | 5 | 3.62 | 1.29 | 5 | 0.60 | 0.19 | 5 |
| Liver | 0.08 | 0.00 | 6 | 8.49 | 2.74 | 5 | 0.13 | 0.01 | 5 | 0.49 | 0.24 | 5 | 0.32 | 0.17 | 5 |

TABLE 6
Serum and tissueGB3 and lysoGb3 substrates at 4 weeks post IV administration From Study 2.

| | WT:WT Veh | | | rAAV9-null | | | rAAV9-WT | | |
|---|---|---|---|---|---|---|---|---|---|
| | Gb3 ug/mL | | | Gb3 ug/mL | | | Gb3 ug/mL | | |
| Serum, | Serum or mg | | | Serum or mg | | | Serum or mg | | |
| Tissue | of Prot | SEM | N | of Prot | SEM | N | of Prot | SEM | N |
| Serum | 0.1 | 0.0 | 6 | 9.9 | 0.7 | 7 | 1.4 | 0.1 | 7 |
| Kidney | 2.3 | 0.5 | 6 | 17.0 | 1.2 | 7 | 3.5 | 0.6 | 7 |
| Heart | 0.0 | 0.0 | 6 | 35.6 | 3.0 | 7 | 10.8 | 0.8 | 7 |
| Liver | 0.0 | 0.0 | 6 | 28.9 | 2.2 | 7 | 0.1 | 0.0 | 7 |
| | rAAV9-40 | | | rAAV9-41 | | | rAAV9-42 | | |
| | Gb3 ug/mL | | | Gb3 ug/mL | | | Gb3 ug/mL | | |
| Serum, | Serum or mg | | | Serum or mg | | | Serum or mg | | |
| Tissue | of Prot | SEM | N | of Prot | SEM | N | of Prot | SEM | N |
| Serum | 1.0 | 0.1 | 8 | 1.5 | 0.1 | 7 | 1.0 | 0.1 | 7 |
| Kidney | 2.5 | 0.6 | 8 | 8.4 | 2.3 | 7 | 2.4 | 0.4 | 7 |
| Heart | 2.7 | 0.7 | 8 | 4.8 | 1.6 | 7 | 1.9 | 0.2 | 7 |
| Liver | 0.1 | 0.0 | 8 | 0.1 | 0.0 | 7 | 0.1 | 0.0 | 7 |
| | WT:WT Veh | | | rAAV9-null | | | rAAV9-WT | | |
| | LysoGb3 | | | LysoGb3 | | | LysoGb3 | | |
| Serum, | ng/mL Serum | | | ng/mL Serum | | | ng/mL Serum | | |
| Tissue | or mg of Prot | SEM | N | or mg of Prot | SEM | N | or mg of Prot | SEM | N |
| Serum | 1.5 | 0.1 | 6 | 568.1 | 33.0 | 7 | 11.8 | 1.1 | 7 |
| Kidney | 0.1 | 0.0 | 6 | 16.9 | 1.2 | 7 | 0.5 | 0.1 | 7 |
| Heart | 0.0 | 0.0 | 6 | 29.9 | 2.5 | 7 | 6.4 | 0.8 | 7 |
| Liver | 0.1 | 0.0 | 6 | 115.4 | 8.2 | 7 | 0.3 | 0.0 | 7 |
| | rAAV9-40 | | | rAAV9-41 | | | rAAV9-42 | | |
| | LysoGb3 | | | LysoGb3 | | | LysoGb3 | | |
| Serum, | ng/mL Serum | | | ng/mL Serum | | | ng/mL Serum | | |
| Tissue | or mg of Prot | SEM | N | or mg of Prot | SEM | N | or mg of Prot | SEM | N |
| Serum | 2.0 | 0.2 | 7 | 2.7 | 0.6 | 6 | 2.1 | 0.1 | 7 |
| Kidney | 0.2 | 0.1 | 8 | 0.4 | 0.2 | 7 | 0.1 | 0.0 | 7 |
| Heart | 1.0 | 0.8 | 8 | 1.7 | 1.1 | 7 | 0.2 | 0.0 | 7 |
| Liver | 0.2 | 0.1 | 8 | 0.2 | 0.1 | 7 | 0.1 | 0.0 | 7 |

TABLE 7
Comparison of tissueGB3 substrate reduction in percentage of G3Stg/GLAko
Null control group (rAAV9-MY011) at 4 weeks post administration of rAAV9
test articles from 2 studies (Study 1 and Study 2) at indicated dose
Study 1

| Tissue % GB3 reduction | | | | |
|---|---|---|---|---|
| from Null group at | | | | |
| 2.5e11 vg/kg dose | rAAV9-WT | rAAV9-A | rAAV9-005 | rAAV9-D |
| Kidney | 74% | 96% | 95% | 93% |
| Heart | 58% | 93% | 86% | 92% |
| Liver | 99% | 99% | 99% | 99% |

Study 2

| | rAAV9-WT | rAAV9-40 | rAAV9-41 | rAAV9-42 |
|---|---|---|---|---|
| Kidney | 79% | 86% | 51% | 86% |
| Heart | 70% | 92% | 87% | 95% |
| Liver | 99.7% | 99.7% | 99.6% | 99.6% |

### Example 13—In Vitro Assessment of Plasmids Expressing Codon-Optimized Engineered α-GAL Variants

This Example examined the α-GAL activity of various codon optimized α-GAL variants in two different cell lines.

The DNA sequence of engineered α-GAL variants D and 004 were further codon optimized to improve expression from human liver, kidney and heart tissues. Up to six individual DNA sequences were each generated for the engineered α-GAL variants D and 004. These were then incorporated into plasmids and used for transfection.

Huh7 (human hepatoma) or HEK293 (human embryonic kidney) cells were transfected with plasmids expressing different α-GAL variants using lipofectamine 3000 reagent kit (Thermo Fisher Scientific) as per manufacturer's instructions. Briefly, cells were seeded at 125,000 cells per well in a 12 well plate format with 1 mL per well growth media and maintained at 37 C, 5% CO2 overnight. Next day, a fresh media was added (1 mL per well) before transfection. For each plasmid, 1 μg of plasmid DNA was added to 2 ul of P3000 reagent, 1.5 μl of lipofectamine 3000 and enough OptiMEM media to make up 100 ul and incubated at room temperature for 10-15 mins. This mixture was then added to the cells and incubated at 37 C, 5% CO2 overnight. Next day, media was refreshed, and cells incubated for another day before collecting the supernatant for α-GAL activity analysis.

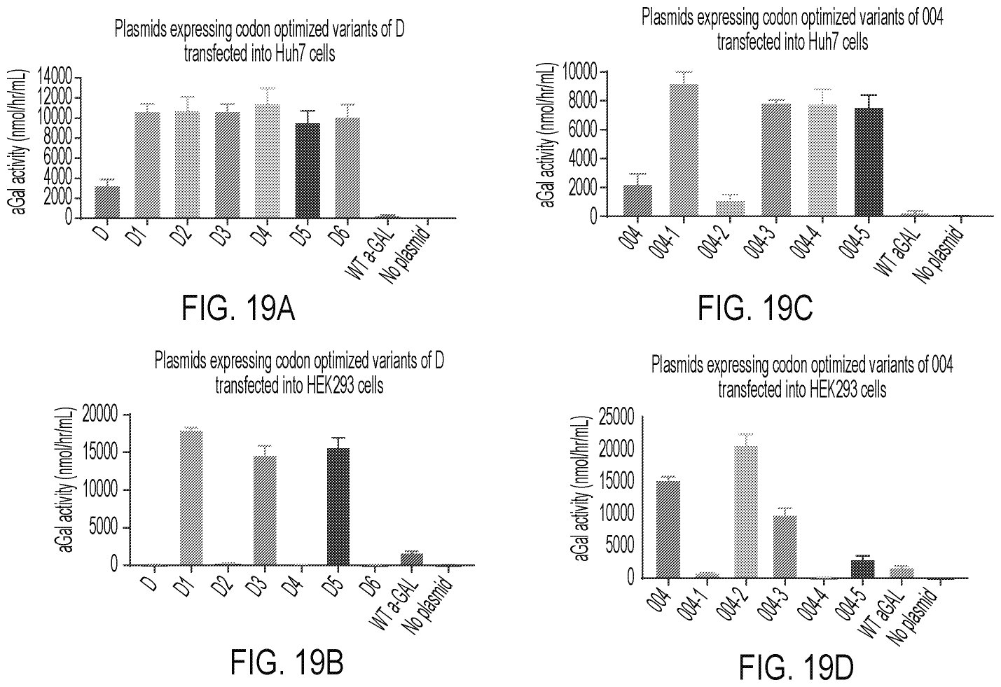

Supernatants from the transfected cells were collected and assayed for α-GAL enzyme activity. All the codon optimized variants of D resulted in significantly higher α-GAL activity compared to either WT α-GAL or non-optimized enzyme D in Huh7 cells. (FIG. 19A). In contrast, in HEK293 cells, only 3 of the optimized variants showed superior activity in supernatant (FIG. 19C). In case of engineered variant 004, only one codon optimized variant showed relatively good activity in both Huh7 and HEK293 cells (FIG. 19B, 19D). The plasmids expressing the two of the codon optimized engineered variants were then packaged into rAAV9 virus and tested in vivo, as described in Example 13.

### Example 14—In Vivo Assessment of Plasmids Expressing Codon-Optimized Engineered α-GAL Variants

This Example examined the α-GAL activity of various codon optimized α-GAL variants in Fabry model mice (G3Stg/GLAko).

10-12 weeks old G3Stg/GLAko male mice were administered once with rAAV9-D3 and rAAV9-004-3, at 4 different doses 2.5×10⁸, 2.5×10⁹, 2.5×10¹⁰ and 2.5×10¹¹ vg/kg, or with a null control rAAV9-NULL at 2.5×10¹¹ vg/kg and monitored for 4 weeks post dose. WT:WT and WT:CAR sibling mice with the same genetic background were used as control and were administered with vehicle only. Mice were assigned to each test article group in a semi-randomized process based on pre-dose body weights to ensure balanced groups. Serum was collected during the study at multiple time points. Blood was collected via retro-orbital or tail vein bleed during the study and via cardiac puncture at termination and processed to collect serum. At the end of the study, terminal serum was collected, and mice were perfused for collection of organs, including liver, kidney and heart then snap frozen in dry ice and stored at −80° C. Analytical evaluations included measurement of α-GAL enzyme activity, and analyses of substrate levels in serum and various tissues.

Tissues were homogenized in lysis buffer containing 10 mM HEPES with 0.5% Triton-X 100 and 1.5× Halt protease inhibitor cocktail, EDTA free, centrifuged and supernatant collected for analytical assays. Alpha galactosidase activity in supernatant or serum was measured using a fluorescent substrate. Briefly, 2 μl of biological samples were incubated with 15 uL 4-MU-α-gal substrate solution (Research Products International Company, catalog #M65400) with α-galactosidase B inhibitor (N-acetyl-D-galactosamine, Sigma catalog #A-2795) at 37° C. for 60 minutes. The enzymatic reaction is stopped by addition of 200 μL glycine carbonate stop solution, pH 10.7. The 4-MU product is measured at the excitation wavelength 360 nm and emission wavelength 465 nm by a fluorescence plate reader. The concentrations of 4-MU in testing samples are calculated from the 4-MU calibration curve in the same plate. Tissue activity is normalized to total protein concentration determined by BCA assay (Thermo Scientific, catalog #23225).

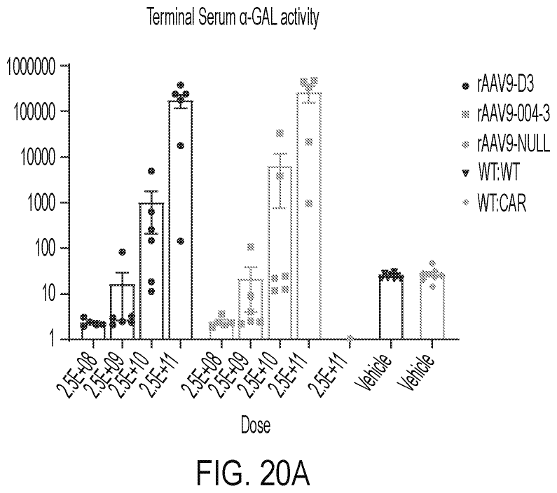

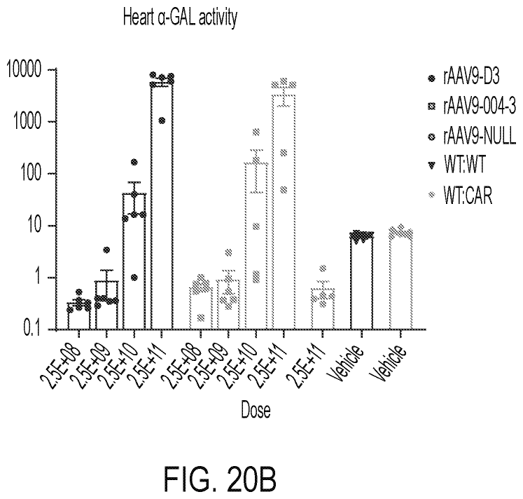

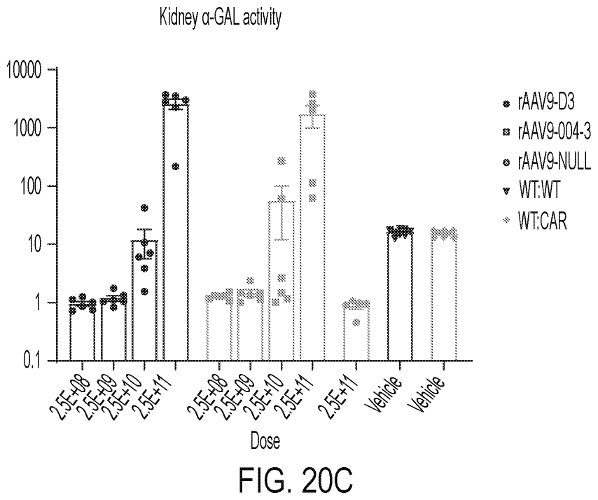

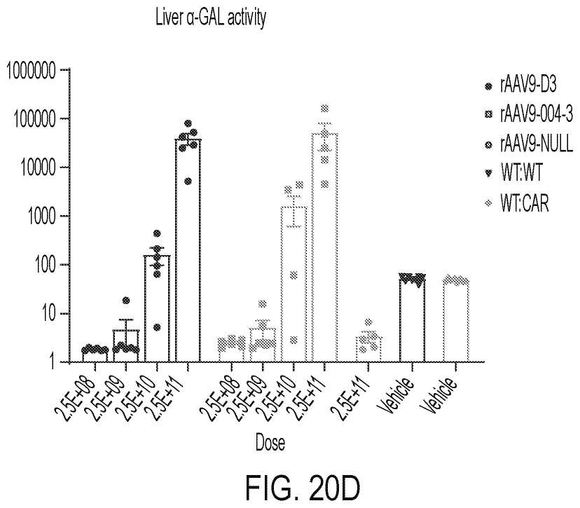

Dose dependent expression of α-GAL was observed in G3Stg/GLAko mice after intravenous administration of rAAV9-D3 and rAAV9-004-3 at doses ranging from 2.5e8 vg/kg to 2.5e11 vg/kg to male G3Stg/GLAko mice resulted in sustained higher α-GAL activity in serum over the 4-week duration of the study (FIG. 20A). The α-GAL activity in serum of animals dosed with rAAV9-D3 and 004-3 at 2.5e11 vg/kg dose was more than 2 logs in order of magnitude higher above normal α-GAL activity in WT mice, as measured in the WT animals treated with vehicle. The G3Stg/GLAko mice treated with rAAV9-NULL had undetectable levels of α-GAL in circulation. Dose dependent increase in α-GAL activity was observed in tissues (kidney, heart and liver) in animals treated with rAAV9-D3 and 004-3 (FIG. 20B-20D). Significant α-GAL activity above normal WT levels was observed at doses at or above 2.5e10 vg/kg of rAAV9-D3 and rAAV9-004-3.

### Example 15—In Vivo Assessment of GB3 and lysoGb3 Substrates with Plasmids Expressing Codon-Optimized Engineered α-GAL Variants

This Example examined the reduction of GB3 and lysoGb3 substrates in various codon optimized α-GAL variants in Fabry model mice (G3Stg/GLAko).

As explained in Example 14, G3Stg/GLAko male mice were administered with rAAV9-D3 and rAAV9-004-3, at 4 different doses. Additionally, tissues were homogenized as previously mentioned in Example 14. Substrates from serum and tissue samples were analyzed using an LC-MS method. Samples were extracted first using Chloroform:Methanol (v/v 2:1) and formic acid before running in HPLC and LC-MS/MS (Applied Biosystem API5000, Turbo Ion Spray Ionization, positive-ion mode).

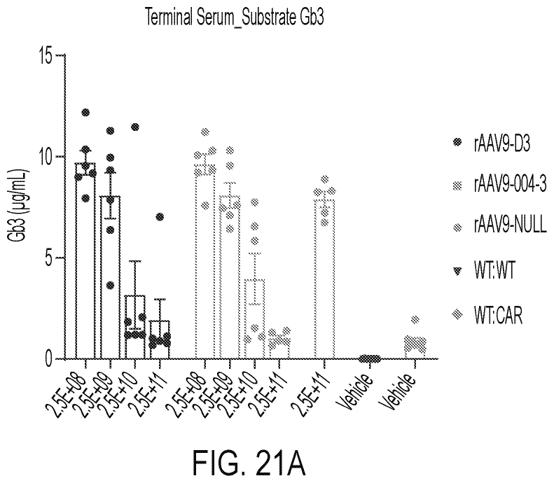

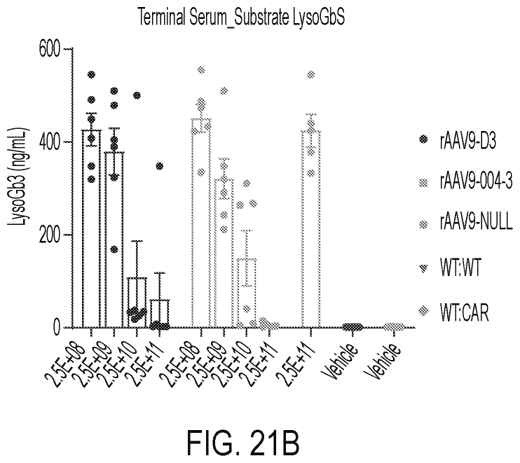

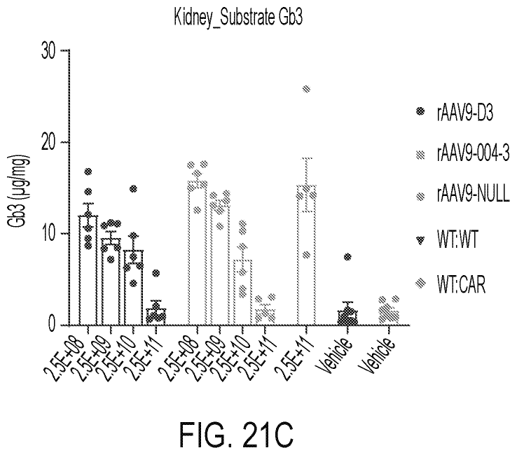

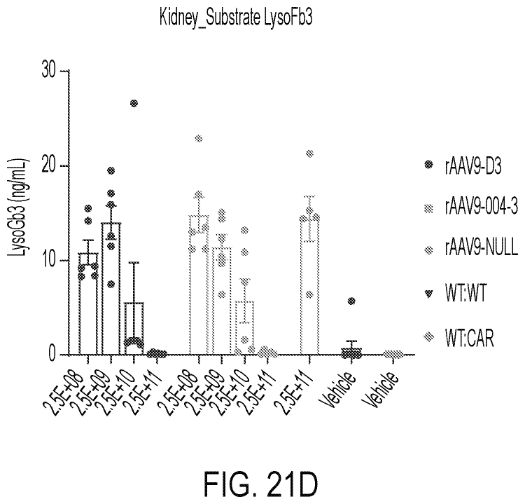

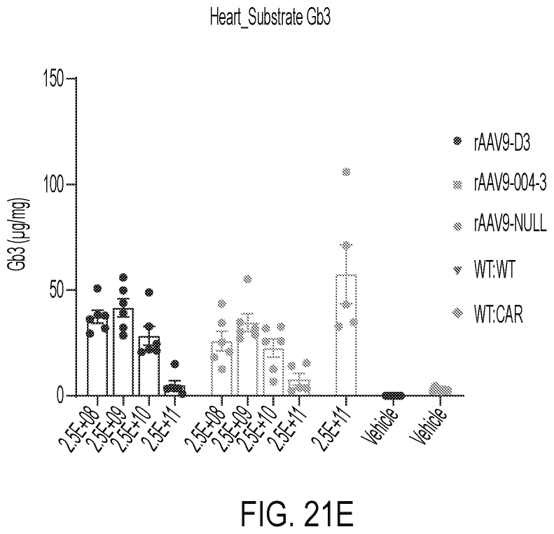

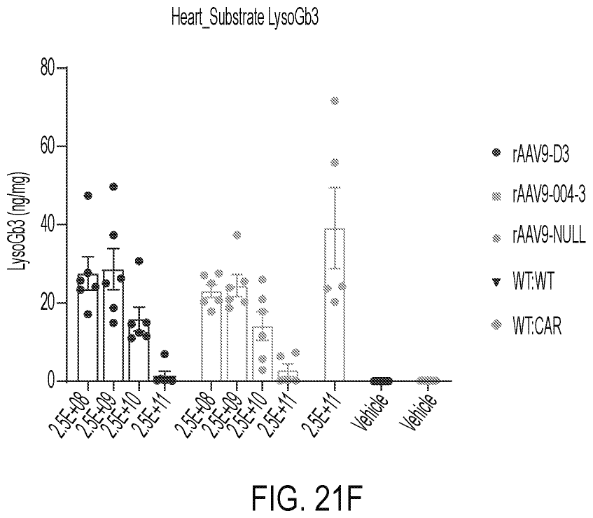

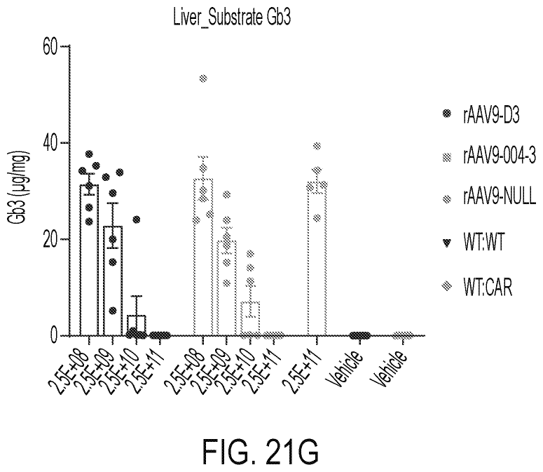

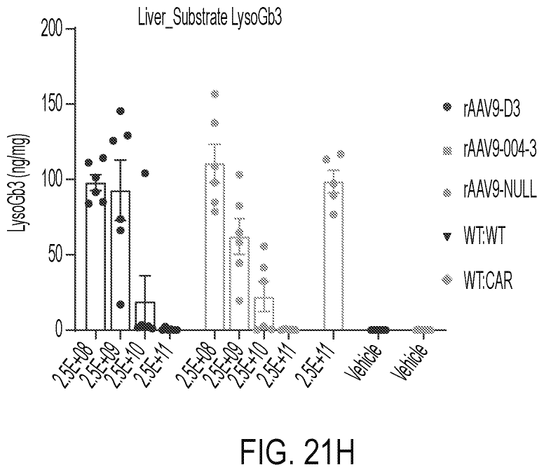

It was observed that both GB3 and lysoGb3 substrates were reduced in a dose dependent manner in mice treated with rAAV9-D3 and rAAV9-004-3 compared to rAAV9-null control (FIG. 21A-21H). At the highest dose of 2.5e11 vg/kg, treatment of animals with either rAAV9-D3 or rAAV9-044-3 resulted in decreasing the levels of GB3 and lysoGb3 substrate to normal WT levels in the key target tissues such as the kidney and the heart. Similar trends were observed for the liver and serum.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the following claims:

1. A recombinant adeno-associated virus (rAAV) vector packaged in an AAV capsid having broad tissue tropism, said vector comprising
a. a 5′ inverted terminal repeat (ITR);
b. a ubiquitous promoter;
c. a nucleotide sequence encoding α-GAL enzyme;
d. a poly A; and
e. a 3′ ITR.

2. A recombinant adeno-associated virus (rAAV) vector packaged in an AAV capsid having broad tissue tropism, said vector comprising
a. a 5′ inverted terminal repeat (ITR);
b. a ubiquitous promoter;
c. a nucleotide sequence encoding α-GAL enzyme;
d. a woodchuck hepatitis virus posttranscriptional regulatory element (WPRE);
e. a poly A; and
f. a 3′ ITR.

3. The recombinant rAAV vector of claim 1 or 2, wherein the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, or AAV9 capsid.

4. The recombinant rAAV vector of claim 3, wherein the wide-tropism AAV capsid is AAV9.

5. The rAAV vector of claim 1, wherein the ubiquitous promoter is selected from chicken R actin (CBA) promoter, EF-1α promoter, PGK promoter, UBC promoter, LSE beta-glucuronidase (GUSB) promoter, or ubiquitous chromatin opening element (UCOE) promoter.

6. The rAAV vector of claim 1, wherein the ubiquitous promoter comprises a cyto-megalo-virus (CMV) enhancer, chicken beta actin promoter, and a rabbit beta globin intron.

7. The rAAV vector of claim 1, wherein the ubiquitous promoter comprises a shortened EF-1α promoter and one or more introns.

8. The rAAV vector of claim 7, wherein the one or more introns are from chicken R-actin and/or rabbit β-globin genes.

9. The rAAV vector of claim 4, wherein the AAV9 capsid is naturally occurring or modified.

10. The rAAV vector of claim 2, wherein the WPRE sequence is modified.

11. The rAAV vector of claim 10, wherein the WPRE sequence is WPRE mut6delATG.

12. The rAAV vector of claim 1 or 2, wherein the poly A is bovine growth hormone (BGH) poly A.

13. The rAAV vector of any one of the proceeding claims, wherein the nucleotide sequence encoding α-GAL enzyme is codon optimized.

14. The rAAV vector of claim 13, wherein the nucleotide sequence encoding α-GAL enzyme is codon optimized for human cells.

15. The rAAV vector of any one of claims 1-13, wherein the α-GAL enzyme has an unmodified sequence.

16. A method of treating Fabry disease, the method comprising administering to a subject in need thereof a recombinant adeno-associated viral vector (rAAV) of any one of the preceding claims.

17. A pharmaceutical composition comprising the rAAV vector of any one of claims 1-15.

18. A cell comprising the rAAV vector of any one of claims 1-15.

19. A method of treating Fabry disease, the method comprising administering to a subject in need thereof a recombinant adeno-associated viral vector (rAAV) packaged in a capsid with broad tissue tropism, the vector comprising:
a. a 5′ inverted terminal repeat (ITR);
b. a ubiquitous promoter comprising a cyto-megalo-virus (CMV) enhancer, chicken beta actin promoter, and a rabbit beta globin intron;
c. a nucleotide sequence encoding α-GAL enzyme;
d. a poly A; and
e. a 3′ ITR.

20. A method of treating Fabry disease, the method comprising administering to a subject in need thereof a recombinant adeno-associated viral vector (rAAV) packaged in a capsid with broad tissue tropism, the vector comprising:
a. a 5′ inverted terminal repeat (ITR);
b. a ubiquitous promoter comprising a cyto-megalo-virus (CMV) enhancer, chicken beta actin promoter, and a rabbit beta globin intron;
c. a nucleotide sequence encoding α-GAL enzyme;
d. a woodchuck hepatitis virus posttranscriptional regulatory element (WPRE);
e. a poly A; and
f. a 3′ ITR.

21. The method of claim 19 or 20, wherein the AAV capsid is a wide-tropism AAV capsid selected from an AAV1 capsid, AAV2 capsid, AAV3 capsid, AAV4 capsid, AAV5 capsid, AAV6 capsid, AAV7 capsid, AAV8 capsid, or AAV9 capsid.

22. The recombinant rAAV vector of claim 21, wherein the wide-tropism AAV capsid is AAV9.

23. The method of claim 19 or 20, wherein the nucleotide sequence encoding α-GAL enzyme is codon optimized.

24. The method of claim 19 or 20, wherein the nucleotide sequence encoding α-GAL enzyme is engineered.

25. The method of claim 24, wherein the nucleotide sequence encoding α-GAL enzyme is engineered and codon optimized.

26. The method of any one of claims 19-22, wherein the α-GAL enzyme has an unmodified sequence.

27. The method of claim 20, wherein the WPRE is WPRE mut6delATG.

28. The method of claim 20, wherein the poly A is a bovine growth hormone (BGH) poly A.

29. The method of any one of claims 19-28, wherein the rAAV vector is administered by intravenous, subcutaneous, or transdermal administration.

30. The method of claim 29, wherein the transdermal administration is by gene gun.

31. The method of any one of claims 19-30, wherein the rAAV vector is episomal following administration.

32. The method of claim 19 or 20, wherein the rAAV vector achieves a therapeutic effect at a lower dose than a rAAV vector comprising an AAV1 capsid, AAV2 capsid, AAV3 capsid, AAV4 capsid, AAV5 capsid, AAV6 capsid, AAV7 capsid, or AAV8 capsid which is specifically targeted to the liver using a liver-specific promoter.

33. The method of any one of claims 19-32, wherein following administration of the rAAV vector, the subject has detectable α-GAL in the serum for at least 5 weeks, 10 weeks, 15 weeks, 26 weeks, 1 year, 5 years, 10 years or 15 years.

34. The method of claim 33, wherein the subject has detectable α-GAL in the serum for greater than 15 weeks.

35. The method of any one of claims 19-34, wherein administration results in α-GAL enzyme expression in one or more of liver, kidney, heart, and gastrointestinal tract, of the subject.

36. The method of any one of claims 19-34, wherein administration of the rAAV vector results in reduced levels of globotriaosylceramide (gb3) in one or more of liver, heart, kidney and GI tract of the subject.

37. A method of expressing α-GAL enzyme in a cell, the method comprising administering a rAAV vector packaged in an AAV9 capsid, said vector comprising:
a. a 5′ inverted terminal repeat (ITR);
b. a ubiquitous promoter comprising a cyto-megalo-virus (CMV) enhancer, chicken beta actin promoter, and a rabbit beta globin intron;
c. a nucleotide sequence encoding α-GAL enzyme;
d. a bovine growth hormone (BGH) poly A; and
e. a 3′ ITR.

38. A method of expressing α-GAL enzyme in a cell, the method comprising administering a rAAV vector packaged in an AAV9 capsid, said vector comprising:
a. a 5′ inverted terminal repeat (ITR);
b. a ubiquitous promoter comprising a cyto-megalo-virus (CMV) enhancer, chicken beta actin promoter, and a rabbit beta globin intron;
c. a nucleotide sequence encoding α-GAL enzyme;
d. a woodchuck hepatitis virus posttranscriptional regulatory element (WPRE) having mut6delATG mutation;
e. a bovine growth hormone (BGH) poly A; and
f. a 3′ ITR.

39. A recombinant adeno-associated virus (rAAV) vector packaged in an AAV capsid having broad tissue tropism, said vector comprising
a. a 5′ inverted terminal repeat (ITR);
b. a liver specific promoter;
c. a nucleotide sequence encoding α-GAL enzyme;
d. a poly A; and
e. a 3′ ITR.

40. A recombinant adeno-associated virus (rAAV) vector packaged in an AAV capsid having broad tissue tropism, said vector comprising
a. a 5′ inverted terminal repeat (ITR);
b. a liver-specific promoter;
c. a nucleotide sequence encoding α-GAL enzyme;
d. a woodchuck hepatitis virus posttranscriptional regulatory element (WPRE);
e. a poly A; and
f. a 3′ ITR.

41. The vector of claim 39 or 40, wherein the nucleotide sequence encoding α-GAL enzyme is codon optimized.

42. The vector of claim 39 or 40, wherein the nucleotide sequence encoding α-GAL enzyme is engineered.

43. The vector of claim 39 or 40, wherein the nucleotide sequence encoding α-GAL enzyme is both codon optimized and engineered.


SEQUENCE LISTING (77 sequences)

SEQ ID NO: 1 - DNA, 130 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 1881 bp, Artificial Sequence
  Synthetic polynucleotide
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatca

SEQ ID NO: 3 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 4 - DNA, 592 bp, Artificial Sequence
  Synthetic polynucleotide
aatcaacctctggattacaaaatttgtgaaagattgactggtattcttaactttgttgctccttttacgctttgtggatacgctgctttattgcctttgtatcttgctattgcttcccgtttggctttcattttctcctccttgtataaatcctggttgctgtctctttttgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatc

SEQ ID NO: 5 - DNA, 234 bp, Artificial Sequence
  Synthetic polynucleotide
cctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaa

SEQ ID NO: 6 - DNA, 133 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagaga

SEQ ID NO: 7 - protein, 398 aa, Artificial Sequence
  Synthetic polypeptide
LDNGLARTPTMGWLHWERFMCNLDCQEEPDSCISEKLFMEMAERMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYEWTSRLKSHINPTGTVLLQLENTMQMSLKDLL

SEQ ID NO: 8 - protein, 398 aa, Artificial Sequence
  Synthetic polypeptide
LDNGLARTPPMGWLHWERFMCNLDCQEEPDSCISEKLFEEMAERMVTEGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRSYTIPVASLGKGVACNPACFITQLLPVKRKLGFYEATSRLRSHINPTGTVLLQLENTMQTSLKDLL

SEQ ID NO: 9 - protein, 398 aa, Artificial Sequence
  Synthetic polypeptide
LDNGLARTPPMGWLHWERFMCNLDCQEEPDSCISEKLFEEMAERMVTEGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRSYTIPVASLGKGVACNPACFITQLLPVKRKLGFYEATSRLRSHINPTGTVLLQLENTMQTSLKDLL

SEQ ID NO: 10 - protein, 398 aa, Artificial Sequence
  Synthetic polypeptide
LDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRGYTIPVASLGKGVACNPACFITQLLPVKRKLGFYEATSRLRSHINPTGTVLLQLENTMQTSLKDLL

SEQ ID NO: 11 - protein, 398 aa, Artificial Sequence
  Synthetic polypeptide
LDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRGYTIPVAKLGKGVACNPACFITQLLPVKRKLGFYEATSRLRSHINPTGTVLLQLENTMQTSLKDLL

SEQ ID NO: 12 - protein, 398 aa, Artificial Sequence
  Synthetic polypeptide
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSFNQERIVDVAGPGGWNDPDMLVIGNFGLSWDQQVTQMALWAIMAAPLFMSNDLRHISPQAKALLQDKDVIAINQDPLGKQGYQLRQGDNFEVWERPLSGLAWAVAVINRQEIGGPRSYTIAVASLGGGVACNPACFITQLLPVKRKLGLYEWTSRLKSHINPTGTVLLQLENTMQMSLKDLL

SEQ ID NO: 13 - protein, 398 aa, Artificial Sequence
  Synthetic polypeptide
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRKGDNFEVWERPLSGDAWAVAMINRQEIGGPRSYTIPVASLGKGVACNPACFITQLLPVKRKLGFYEWTSRLRSHINPTGTVLLQLENTMQMSLKDLL

SEQ ID NO: 14 - protein, 398 aa, Artificial Sequence
  Synthetic polypeptide
LDNGLARTPTMGWLHWERFMCNLDCQEEPDSCISEKLFMEMAERMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSRNQERIVDVAGPGGWNDPDMLVIGNFGLSWDQQVTQMASWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 15 - protein, 398 aa, Artificial Sequence
  Synthetic polypeptide
LDNGLARTPTMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSRNQERIVDVAGPGGWNDPDMLVIGNFGLSWNQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRKLGFYEWTSRLRSHINPTGTVLLQLENTMQMSLKDLL

SEQ ID NO: 16 - protein, 398 aa, Artificial Sequence
  Synthetic polypeptide
LDNGLARTPTMGWLHWERFMCNLDCQEEPDSCISEKLFEEMAELMVSEGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGDAWAVAMINRQEIGGPRSYTIPVASLGKGVACNPACFITQLLPVKRKLGFYEWTSRLRSHINPTGTVLLQLENTMQMSLKDLL

SEQ ID NO: 17 - protein, 398 aa, Artificial Sequence
  Synthetic polypeptide
LDNGLARTPTMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRKLGFYEATSRLKSHINPTGTVLLQLENTMQMSLKDLL

SEQ ID NO: 18 - DNA, 1197 bp, Artificial Sequence
  Synthetic polynucleotide
ctggataatggattggctagaacacctactatggggtggcttcactgggagaggttcatgtgcaacctcgactgtcaggaagaaccagacagctgcatctccgagaagctgtttatggaaatggccgagcgaatggtgtcagaaggctggaaagatgcaggttacgagtatctgtgtattgacgattgctggatggctccgcaacgggacagtgagggcagacttcaggcagatcctcagcgcttcccacatgggataaggcagctcgccaactacgtccactctaagggactgaaactgggcatctatgctgacgtggggaataagacctgtgcgggatttcccggtagcttcggctactacgacattgatgcccagacctttgccgattggggagttgacctcctcaaattcgatggctgctattgtgactctttggagaacctggcagacgggtacaagcatatgtccctggccctgaatcggacaggtagacccatcgtgtatagttgcgaatggcccctttacatgtggccttttcaaaagccaaactacactgagattcgccagtattgcaatcactggaggaacttcgctgatatcgatgactcatgggcgagcatcaaatccatattggattggacctctcggaatcaggagcgcattgtagacgtcgcaggacccggcggctggaacgaccctgatatgctggtgatcgggaattttggtcttagctgggaccagcaagttacgcagatggctctgtgggcaattatggcagccccactcttcatgtccaacgatctgcgacacatctctcctcaagctaaggctctgctgcaggacaaagatgtgattgccatcaatcaggacccactcggaaagcagggctatcagctgagaaaaggcgacaacttcgaagtctgggaaaggccactttcaggagacgcatgggctgtggccataataaaccggcaagagattggtgggcccaggagctacacaatccccgttgccagtttgggcaagggagtggcgtgtaatcctgcctgctttatcactcagctgctcccagtcaaaagacagctggggttctatgagtggacctcccgcctcaagagccatattaatcccacaggtaccgtactgctgcaacttgaaaacacgatgcagatgagtttgaaggacctcctgtag

SEQ ID NO: 19 - DNA, 1197 bp, Artificial Sequence
  Synthetic polynucleotide
ctggataatggattggctagaacacctcctatggggtggcttcactgggagaggttcatgtgcaacctggactgtcaggaagaaccagacagctgcatctccgagaagctctttgaggaaatggccgaacgaatggtgactgagggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaatagaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgctcctatacaattcctgtagcatctcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 20 - DNA, 1197 bp, Artificial Sequence
  Synthetic polynucleotide
ctggataatggattggctagaacacctcctatggggtggcttcactgggagaggttcatgtgcaacctggactgtcaggaagaaccagacagctgcatctccgagaagctctttgaggaaatggccgaacgaatggtgactgagggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgctcctatacaattcctgtagcatctcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 21 - DNA, 1197 bp, Artificial Sequence
  Synthetic polynucleotide
ctggataatggattggctagaacacctcctatggggtggcttcactgggagaggttcatgtgcaacctggactgtcaggaagaaccagacagctgcatctccgagaagctctttgaggaaatggccgaacgaatggtgactgacggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgcggttatacaattcctgtagcatctcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 22 - DNA, 1197 bp, Artificial Sequence
  Synthetic polynucleotide
ctggataatggattggctagaacacctcctatggggtggcttcactgggagaggttcatgtgcaacctggactgtcaggaagaaccagacagctgcatctccgagaagctctttgaggaaatggccgaacgaatggtgactgacggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgcggttatacaattcctgtagcaaagcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 23 - DNA, 1197 bp, Artificial Sequence
  Synthetic polynucleotide
ctggataatggattggctagaacacctcctatggggtggcttcactgggagaggttcatgtgcaacctggactgtcaggaagaaccagacagctgcatctccgagaagctctttgaggaaatggccgaacgaatggtgactgacggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcgggatatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggccctcttttcatgagcaacgatctgcgcgcgatctcaccacaggcaaaggccctgctccaagacacggacgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgcggttatacaattcctgtagcaaagcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 24 - DNA, 1197 bp, Artificial Sequence
  Synthetic polynucleotide
ttggacaatgggctggccaggacacctactatgggctggctccactgggagcgctttatgtgtaacctcgactgccaagaggagccagactcatgcatctctgagaagttgttcatggagatggctgagctgatggtgagcgaagggtggaaggatgcgggctatgagtatctctgtattgatgactgctggatggctccacagcgcgacagtgaaggccggctccaggccgatcctcagcggttcccccacggtatcagacaactggcgaattacgtgcactcaaaaggccttaagctgggtatatatgctgatgtgggtaataaaacatgtgcaggcttcccaggctcttttgggtactatgacatcgacgcccagacttttgcggactggggcgtggacctgctcaagtttgacggatgttactgtgactcccttgagaacctggccgacgggtacaagcatatgtcactggccctgaatcggacaggccgatccatcgtatactcttgcgagtggcctctgtacatgtggcccttccagaagcccaactatacagaaatcaggcaatactgcaaccattggcggaacttcgcagacatagacgacagctgggctagcattaagtctattctggattggaccagtttcaatcaagaaaggattgtcgatgtcgcaggcccaggaggttggaatgacccagacatgctcgtgattggaaatttcggtctgtcatgggaccaacaggtgactcagatggctctgtgggcaatcatggcagcaccactgttcatgagcaatgatttgcgacacatctcccctcaggcgaaagcccttctgcaggataaggacgttatcgccattaaccaggacccgctcggtaagcaagggtaccagttgcgccagggagacaatttcgaggtctgggaacgacccctgtctggactcgcttgggccgtagccgtaattaaccgacaagaaatcggcggaccgcggagctataccatagctgtcgcctccctcggcgggggcgtagcttgtaacccggcttgtttcataacccagctgctgcccgtcaagaggaaactggggctttatgagtggacaagtcggttgaagtctcatattaacccgacagggactgttctcctccagctggaaaacacaatgcagatgagcctgaaggatctcttgtga

SEQ ID NO: 25 - DNA, 1198 bp, Artificial Sequence
  Synthetic polynucleotide
tttggacaatgggctggccaggacacctactatgggctggctccactgggagcgctttatgtgtaacctcgactgccaagaggagccagactcatgcatctctgagaagttgttcatggaaatggccgagcttatggtctcagaggggtggaaagacgccggatatgagtatctgtgcatcgacgattgctggatggccccccaacgcgattccgaaggccgcttgcaggctgatccgcagcgctttcctcacgggatccgccaattggcaaattacgtgcactcaaaggggctcaagttgggcatctacgcagacgtgggaaacaagacatgtgctggatttcccgggagtttcggttattatgacattgacgcacagacctttgctgattggggggtcgacctcctgaagtttgacggttgttattgcgacagccttgaaaatctggccgacggttacaaacacatgtcccttgcactgaatagaactggtcggtccatcgtctatagttgtgagtggccgctttacatgtggccttttcagaaacccaactacaccgagattcggcaatattgcaatcactggcgaaatttcgcagatatcgatgattcttgggctagtattaaatccatcctggattggacatcattcaaccaggagcgcatcgtggacgttgctggacctggcgggtggaatgatccagacatgcttgtgatcggaaacttcggtctctcttggaaccagcaagtcactcaaatggcactctgggcaattatggccgcccccctctttatgtccaacgatctgaggcatatcagtcctcaggctaaagccctgctgcaagacaaggatgtgattgctatcaaccaagatccccttggtaaacaggggtaccagttgcgcaaaggcgacaattttgaggtgtgggaaaggccactttcaggcgatgcatgggccgttgcaatgatcaacaggcaagaaattggcggacccaggagctatacaataccagtggcgtcactgggtaagggagtcgcctgtaaccccgcatgcttcattactcaacttctgccagtgaaacgaaaactcggattctatgagtggacctcaagactgaggtctcatattaacccgacagggactgttctcctccagctggaaaacacaatgcagatgagcctgaaggatctcttgtga

SEQ ID NO: 26 - DNA, 1197 bp, Artificial Sequence
  Synthetic polynucleotide
ttggacaatgggctggccaggacacctactatgggctggctccactgggagcgctttatgtgtaacctcgactgccaagaggagccagactcatgcatctctgagaagttgttcatggagatggctgaacgcatggtttctgagggatggaaagatgcaggctacgagtacctgtgtatagacgattgttggatggccccacagcgggattcagaaggtagactccaggcggatccccagagattcccacatggaatcagacagctggccaattacgtccattccaaaggccttaagttgggtatttacgccgacgtaggcaacaagacttgtgccggatttcccggcagtttcggatactatgacattgatgcacagactttcgctgactggggggtggacttgctcaaatttgatggctgttattgcgatagcctcgaaaatctggctgatggctacaagcacatgtcactcgctctcaaccgcactgggcgctctatagtttactcctgcgagtggcctctgtatatgtggccgttccagaaacccaattacacagaaataaggcagtattgcaatcactggcgcaactttgctgatattgatgattcctgggcctccataaagagtatcttggactggactagtcgcaatcaggaaagaattgtcgacgtcgccggaccaggcggatggaatgatcctgatatgctcgtgatcgggaacttcggactctcatgggaccagcaggtgacccagatggctagttgggctatcatggccgcccctctgtttatgagtaacgacctccgccacatcagcccccaggccaaggcgcttctgcaggataaagatgtcatcgccatcaaccaagatcccctgggcaaacaaggctatcagctgcggaagggagacaattttgaggtgtgggaacgccctttgagcggagacgcctgggctgtggctattataaatcgccaggagattgggggcccgagaagttatactatccccgttgcaagcctgggaaagggcgtcgcctgcaaccctgcctgcttcatcacacagctgcttcctgtcaaacgccaattggggttctacaattggacatccagactcaaatctcatattaacccgacagggactgttctcctccagctggaaaacacaatgcagatgagcctgaaggatctcttgtga

SEQ ID NO: 27 - DNA, 1197 bp, Artificial Sequence
  Synthetic polynucleotide
ttggacaatgggctggccaggacacctactatgggctggctccactgggagcgctttatgtgtaacctcgactgccaagaggagccagactcatgcatctctgagaagttgttcgaggagatggcagaacgaatggtgacagatggatggaaggacgctggctacgagtatctgtgcatagatgattgttggatggcccctcagcgagactcagaggggagactccaggccgacccccagcgatttccacacggaatccggcaactggctaaccatgtgcactcaaaagggctcaagctgggaatttatgctgacgtcgggaacaaaacttgtgcggggtttcccggctccttcggatattacgacatcgacgcccagactttcgcagactggggtgtggacctgcttaagttcgacggctgttactgcgatagtctggaaaacttggctgacggctataagcacatgagtctcgccctgaaccgaacaggcagaagcatagtctactcctgcgaatggccactttacatgtggccattccagaaacctaattataccgagatcagacaatactgtaaccattggcgaaacttcgccgacattgacgatagttggaagtcaatcaagtccatcctggattggacctctaggaaccaggaaaggatcgtggacgtggctggacctggcggatggaacgatccagacatgctcgtgataggaaactttggactgtcatggaatcagcaagtaacacagatggcgctctgggccattatggctgcccccttgtttatgtctaacgacctgaggcatatctctcctcaagccaaggcactcctgcaggacaaggacgttatcgccatcaaccaggacccactgggcaagcagggataccagctgcggaaaggtgataacttcgaggtctgggagcgaccgctttcaggagacgcctgggcagttgcaatcatcaacaggcaagaaattggtgggccacggtcttatactattcccgtggcttctctcggtaagggcgtcgcctgcaaccccgcctgctttatcacccaattgttgcccgttaagagaaaactgggattttacgagtggacatccaggctgcggtctcatattaacccgacagggactgttctcctccagctggaaaacacaatgcagatgagcctgaaggatctcttgtga

SEQ ID NO: 28 - DNA, 1197 bp, Artificial Sequence
  Synthetic polynucleotide
ttggacaatgggctggccaggacacctactatgggctggctccactgggagcgctttatgtgtaacctcgactgccaagaggagccagactcatgcatctctgagaagttgttcgaagagatggctgaactgatggtgtccgaggggtggaaggatgcagggtacgagtacctttgcatagacgattgctggatggcaccacagcgggatagtgagggcaggttgcaggcggacccccaaagatttccacatggcatcagacagctggccaaccacgtgcactctaaaggcctgaagctggggatttacgccgatgtcggtaataaaacatgtgctggtttccccggtagctttggatactacgacatcgacgcccagacatttgctgattggggagtggacctgctcaagttcgacggctgctactgcgattctctggaaaatctggccgatgggtataagcacatgagtcttgctcttaatagaaccggacgctctatagtctattcatgtgagtggccactgtacatgtggccatttcagaaacccaactataccgaaattagacagtattgtaaccactggcggaatttcgccgacatagacgatagctggaagagcatcaagtcaattctcgattggacttcattcaaccaggagcggatcgtcgacgtggccggccctgggggctggaatgatccagatatgctggtgatcggaaactttggtctctcctggaatcaacaggtcactcagatggccctctgggccattatggcagcaccactctttatgagcaacgacctgagacacatttccccacaggccaaggcattgctgcaggacaaagacgtgatcgccattaaccaggacccactgggcaagcaagggtaccaacttaggcagggagataattttgaggtctgggagcgcccactcagcggagacgcctgggccgttgccatgataaacagacaggagattggcggccccagatcctacacaattcctgtcgctagcctgggcaagggggtggcttgtaatcccgcctgctttataacccagctgctgccagtgaaacggaaactcgggttctatgaatggacttctaggctcaggtctcatattaacccgacagggactgttctcctccagctggaaaacacaatgcagatgagcctgaaggatctcttgtga

SEQ ID NO: 29 - DNA, 1197 bp, Artificial Sequence
  Synthetic polynucleotide
ttggacaatgggctggccaggacacctactatgggctggctccactgggagcgctttatgtgtaacctcgactgccaagaggagccagactcatgcatctctgagaagttgttcgaggagatggcagaacgaatggtgacagatggatggaaggacgctggctacgagtatctgtgcatagatgattgttggatggcccctcagcgagactcagaggggagactccaggccgacccccagcgatttccacacggaatccggcaactggctaaccatgtgcactcaaaagggctcaagctgggaatttatgctgacgtcgggaacaaaacttgtgcggggtttcccggctccttcggatattacgacatcgacgcccagactttcgcagactggggtgtggacctgcttaagttcgacggctgttactgcgatagtctggaaaacttggctgacggctataagcacatgagtctcgccctgaaccgaacaggcagaagcatagtctactcctgcgaatggccactttacatgtggccattccagaaacctaattataccgagatcagacaatactgtaaccattggcgaaacttcgccgacattgacgatagttggaagtcaatcaagtccatcctggattggacctctaggaaccaggaaaggatcgtggacgtggctggacctggcggatggaacgatccagacatgctcgtgataggaaactttggactgtcatgggatcagcaagtaacacagatggcgctctgggccattatggctgcccccttgtttatgtctaacgacctgaggcatatctctcctcaagccaaggcactcctgcaggacaaggacgttatcgccatcaaccaggacccactgggcaagcagggataccagctgcggaaaggtgataacttcgaggtctgggagcgaccgctttcaggagacgcctgggcagttgcaatcatcaacaggcaagaaattggtgggccacggtcttatactattcccgtggcttctctcggtaagggcgtcgcctgcaaccccgcctgctttatcacccaattgttgcccgttaagagaaaactgggattttacgaggcgacatccaggctgaagtctcatattaacccgacagggactgttctcctccagctggaaaacacaatgcagatgagcctgaaggatctcttgtga

SEQ ID NO: 30 - protein, 398 aa, Artificial Sequence
  Synthetic polypeptide
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 31 - DNA, 1196 bp, Artificial Sequence
  Synthetic polynucleotide
tggacaatggattggcaaggacgcctaccatgggctggctgcactgggagcgcttcatgtgcaaccttgactgccaggaagagccagattcctgcatcagtgagaagctcttcgaggagatggcagagagaatggtcaccgacggctggaaggatgcaggttatgagtacctctgcattgatgactgttggatggctccccaaagagattcagaaggcagacttcaggcagaccctcagcgctttcctcatgggattcgccagctagctaatcacgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctgggccagtataaagagtatcttggactggacatctagaaaccaggagagaattgttgatgttgctggaccagggggttggaatgacccagatatgttagtgattggcaactttggcctcagctgggaccagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagaaagggagacaactttgaagtgtgggaacgacctctctcaggcgatgcctgggctgtagctatcataaaccggcaggagattggtggacctcgctcttataccatccccgttgcttccctgggtaaaggagtggcctgtaatcctgcctgcttcatcacacagctcctccctgtgaaaaggcagctagggttctataactggacttcaaggttaaagagtcacataaatcccacaggcactgttttgcttcagctagaaaatacaatgcagatgtcattaaaagacttactttaa

SEQ ID NO: 32 - DNA, 1196 bp, Artificial Sequence
  Synthetic polynucleotide
tggacaatggattggcaaggacgcctcccatgggctggctgcactgggagcgcttcatgtgcaaccttgactgccaggaagagccagattcctgcatcagtgagaagctcttcgaggagatggcagagagaatggtcaccgacggctggaaggatgcaggttatgagtacctctgcattgatgactgttggatggctccccaaagagattcagaaggcagacttcaggcagaccctcagcgctttcctcatgggattcgccagctagctaatcacgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataagactggcagacccattgtgtactcctgtgagtggcctctttatatgtggccctttcaaaagcccaattatacagaaatccgacagtactgcaatcactggcgaaattttgctgacattgatgattcctgggccagtataaagagtatcttggactggacatctagaaaccaggagagaattgttgatgttgctggaccagggggttggaatgacccagatatgttagtgattggcaactttggcctcagctgggaccagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagaaagggagacaactttgaagtgtgggaacgacctctctcaggcgatgcctgggctgtagctatcataaaccggcaggagattggtggacctcgctcttataccatccccgttgcttccctgggtaaaggagtggcctgtaatcctgcctgcttcatcacacagctcctccctgtgaaaaggcagctagggttctataactggacttcaaggttaaagagtcacataaatcccacaggcactgttttgcttcagctagaaaatacaatgcagatgtcattaaaagacttactttaa

SEQ ID NO: 33 - protein, 398 aa, Artificial Sequence
  Synthetic polypeptide
LDNGLARTPTMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 34 - protein, 398 aa, Artificial Sequence
  Synthetic polypeptide
LDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 35 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggactggccagaactcctcccatgggctggctgcactgggaaaggtttatgtgtaatctggactgtcaagaggagccagattcctgcatctctgagaagctctttgaagagatggctgagaggatggtgacagatggttggaaggatgctggttatgagtacctgtgcattgatgactgctggatggccccccagagagattcagaaggcagactgcaagcagaccctcagaggttcccccatggcatccgccagcttgccaaccatgtccactccaagggcctgaaactgggtatctatgctgatgtgggcaataagacctgtgctggctttcctggctcctttggctactatgacattgacgcccagacctttgctgactggggtgtggacttgctgaagtttgatggctgctactgtgactccctggagaacctggcagatggatacaagcacatgtctctggctctgaacaagactggcagacccattgtttacagctgtgagtggccactgtacatgtggcccttccagaagcccaactacactgagatcagacagtactgcaatcattggaggaactttgccgacatcgatgattcttgggcctccatcaagagcatcctggactggacatccagaaaccaagaaagaattgtggatgtggctggacctggaggatggaatgatcctgacatgctggtgattggaaattttgggctgtcctgggaccagcaagtgactcagatggccctctgggccatcatggctgggcccctcttcatgagcaatgacctgagggccatttccccccaagccaaggccctgcttcaagacaaagatgtcattgctatcaatcaagatcccctggggaagcaaggctaccagctcagaaaaggagacaacttcgaggtgtgggagagacctctgtctggagatgcctgggctgtggccatcatcaacagacaagagattggtggccccagaggttacaccatccctgttgcttctcttggcaagggtgttgcctgcaacccagcttgcttcatcacccagctgctcccagtgaagaggaagctgggcttctatgaagctacctctaggttgagatcccacatcaaccccactggtactgtgctgctgcagctggaaaacaccatgcagacttccctcaaggacctcctgtga

SEQ ID NO: 36 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggacttgcaagaactcctcccatgggctggctgcactgggagaggtttatgtgtaatctggactgtcaagaagaaccagattcctgcatctctgagaagctctttgaggagatggctgagaggatggtgacagatggatggaaggatgctggttatgaatatctgtgcattgatgactgctggatggcccctcagagagacagtgaaggccgcctgcaagcagacccccagaggttcccccatggaatcagacagttggccaaccatgtccactccaagggcctgaaactgggcatctatgctgatgtgggcaataagacctgtgctggcttccctggctcctttgggtactatgacattgacgcccagacttttgctgactggggtgtggacttgctgaagtttgatggctgctactgtgactccctggaaaacctggcagatggttacaagcacatgtctctggccctgaacaagactggcagacccattgtttacagctgtgagtggccactgtacatgtggcccttccagaagcccaactacactgagatccgccagtactgcaatcattggaggaactttgcagacatcgatgattcttgggccagcatcaagtccatcctggactggacttccagaaaccaagagagaattgtggatgttgctggacctggaggttggaatgatcctgacatgctggtgattggaaattttggactgtcctgggaccagcaagtgactcagatggccctctgggccatcatggctgggcccctcttcatgagcaatgacctgagggccatttccccccaagccaaggctctgcttcaagacaaagatgtcattgctattaatcaagatcccctggggaagcaaggctaccagctcagaaaaggagacaacttcgaggtgtgggaaagacctctgtctggagatgcctgggctgtggctatcatcaacagacaagaaattggtggccccagaggctacaccatccctgtggcctctcttggcaagggtgttgcctgcaacccagcttgcttcatcacccagctgctcccagtgaagaggaagctgggtttctatgaggctacctctaggttgagatcccacatcaatcccactggtacagtgctgctgcagctggagaacaccatgcagacctccctcaaggacctcctttaa

SEQ ID NO: 37 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatggactggcaagaactcctcccatgggctggctgcactgggaaaggtttatgtgtaatctggactgtcaagaggagccagattcctgcatctctgagaagctctttgaagagatggctgagaggatggtgacagatggatggaaggatgctggatatgagtacctgtgcattgatgactgctggatggcccctcagagagacagtgaaggcagactgcaagcagacccccagaggttcccccatggcattaggcagctggccaaccatgtccactccaagggcctgaaactgggcatctatgctgatgtgggcaataagacctgtgctggtttccctggctcctttggatactatgacattgatgcccagacctttgctgactggggtgtggacctgctcaagtttgatggctgctactgtgactccctggagaacctggctgatggttacaagcacatgtctcttgctctgaacaagactggtagacccattgtttacagctgtgagtggccactgtacatgtggcccttccagaagcccaactacactgagatcagacagtactgcaatcattggaggaattttgcagatattgatgattcttgggcctccatcaagagcatcctggactggacttccagaaaccaagaaagaattgtggatgttgctggccctggaggttggaatgaccctgacatgctggtgattggcaactttgggctgtcctgggaccagcaagtgactcagatggccctctgggccatcatggctgggcccctcttcatgtccaatgatctgagggccatttccccccaagccaaggccctgcttcaagacaaagatgtcattgctatcaatcaagatcccctgggcaagcaaggctaccagctcagaaaaggagacaactttgaggtgtgggagagacctctgtctggagatgcctgggctgtggccatcatcaacagacaagagattggtggccccagaggctacaccatccctgtggcttccctggggaagggtgttgcctgcaacccagcttgcttcatcacccagcttctgccagtgaagaggaagctgggcttctatgaagctaccagcagactgagatcccacatcaaccccactggcacagtgctgctgcagctggaaaacaccatgcagacttctctgaaggacctcctgtga

SEQ ID NO: 38 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggactggcaagaactcctcccatgggctggctgcattgggagaggtttatgtgtaatctggactgtcaagaggagccagactcctgcatctctgagaagctctttgaagagatggctgagaggatggtgacagatggttggaaggatgctggatatgagtacctgtgcattgatgactgctggatggccccccagagagacagtgaaggcagactgcaagcagaccctcagaggttcccccatggcattaggcagctggccaaccatgtccactccaagggcctgaaacttggcatctatgctgatgtgggcaataagacctgtgctggcttccctggctcctttggctactatgacattgatgcccagacttttgctgactggggtgtggacctgctcaagtttgatggctgctactgtgactctctggaaaacctggctgatggatacaagcacatgtctttggctctgaacaagactggtaggcccattgtttacagctgtgagtggccactgtacatgtggcccttccagaagcccaactacactgagatcagacagtactgcaaccactggaggaactttgcagatattgatgattcctgggcctccatcaagagcatcctggactggacttccagaaaccaagagagaattgtggatgttgctgggcctggaggatggaatgatcctgacatgctggtgattggaaattttgggctgagctgggaccagcaagtgactcagatggccctctgggccatcatggctggtcccctcttcatgagcaatgacttgagggccatttccccccaagccaaggccctgcttcaagacaaagatgtcattgctatcaatcaagatcccctgggcaagcaaggctaccagttgagaaaaggagacaactttgaggtgtgggaaagacctctgtctggagatgcctgggctgtggccatcatcaacagacaagaaattggtggccccagaggttacaccatccctgtggcttctcttggaaaaggggttgcctgcaatccagcttgcttcatcacccagctcctgccagtgaagaggaagctgggtttctatgaagctacctctaggctcagatcccacatcaaccccactggcacagtgctgctgcagctggagaacaccatgcagacctccctgaaggacctcctttaa

SEQ ID NO: 39 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggactggcaagaactcctcccatgggctggctgcactgggaaaggtttatgtgtaatctggactgtcaagaggagcctgactcctgcatctctgagaagctctttgaagagatggctgagaggatggtgacagatggatggaaggatgctggttatgagtacctgtgcattgatgactgctggatggcccctcagagagattctgaaggcagactgcaagcagacccccagaggttcccccatggcattaggcagctggccaaccatgtccactccaagggcctgaaactgggcatctatgctgatgtgggcaataagacctgtgctggctttcctggctcctttggatactatgacattgatgcccagacctttgctgactggggtgtggacctcctgaagtttgatggctgctactgtgactctctggagaacctggctgatggttacaagcacatgtctcttgctctgaacaagactggtagacccattgtttacagctgtgagtggccactgtacatgtggcccttccagaagcccaactacactgagatcagacagtactgcaatcattggaggaattttgcagatattgatgattcctgggcctccatcaagagcatcctggactggacatccagaaaccaagaaagaattgtggatgtggctgggcctggaggttggaatgacccagacatgctggtgattggcaactttgggctgagctgggaccagcaagtgactcagatggccctctgggccatcatggctggacctctcttcatgtccaatgatctgagagccatttccccccaagccaaggccctgctccaagacaaagatgtcattgctatcaatcaagatcccctggggaagcaaggctaccagctcagaaagggtgacaactttgaggtgtgggagagacctctgtctggagatgcctgggctgtggccatcatcaacagacaagagattggtggccccagaggctacaccatccctgttgcttccctgggaaaaggagtggcctgcaacccagcttgcttcatcacccagcttctgcctgtgaagaggaagctgggcttctatgaagctacctctaggctgaggtcccacatcaaccccactggcacagtgctgctgcagctggaaaacaccatgcagacttccctcaaggacctgctttaa

SEQ ID NO: 40 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggacaatggcctggccagaacccctcccatgggctggctgcactgggagagattcatgtgcaacctggattgccaggaggagccagactcttgcatctctgaaaagctgtttgaggaaatggccgagagaatggtgacagatggatggaaggatgccggatacgagtacctgtgtatcgatgactgttggatggccccccagagagactccgagggccgtctgcaggctgacccacagaggtttcctcatggaattaggcagttggccaaccatgtgcactccaagggactgaagctgggcatctatgccgatgtgggcaacaagacctgtgctggcttcccaggcagctttggctattatgatattgatgcacaaacttttgcagactggggagttgatctgctgaaatttgatgggtgttactgtgactccctggagaacctcgccgacggatacaagcatatgtcccttgctctgaacaagactggcaggcccattgtctactcttgtgagtggccactgtacatgtggcccttccagaagcccaactataccgagattcgccagtactgcaatcactggaggaactttgcagacattgatgacagctgggcctccattaagtctatcctggattggacaagcagaaaccaagagagaattgtggatgtggctggccctggtggttggaatgaccccgatatgctggtgattggcaactttggactgtcttgggaccagcaggtgacccagatggctctgtgggctatcatggccggccccctcttcatgtctaatgacctgagggccatctctcctcaggccaaggcacttctgcaggataaggacgtgattgccatcaatcaggatcctctggggaagcagggctatcagctgagaaagggggacaatttcgaggtctgggagagacccctgagcggcgatgcttgggctgtggccatcatcaatagacaagagattggagggccaagaggctacaccattcctgtggcaagcctaggaaaaggggttgcttgcaatccagcctgtttcatcacccagctgctgcctgttaagagaaagttgggcttctatgaggccacctctaggctgaggtcccatatcaaccccactggcacagtgctgctgcagcttgaaaacaccatgcagacctccctcaaggacctgctgtaa

SEQ ID NO: 41 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggactggcaagaaccccaaccatgggctggctgcactgggaaagattcatgtgtaatctggactgtcaagaggagcccgactcctgcatctctgagaagctcttcatggagatggctgagaggatggtgagtgaaggatggaaggatgctggttatgagtacctgtgcattgatgactgctggatggccccccagagagattcagagggcagactgcaagcagatcctcagaggttcccccatggcatcagacagctggccaactatgtccactccaagggcctgaagctgggtatctatgctgatgtgggcaacaagacctgtgctggctttcctggctcctttggttactatgacattgacgcccagacctttgctgactggggagtggacctgttgaagtttgacggctgctactgtgactctctggagaacctggctgatggctacaagcacatgtctcttgccctgaacagaactggtaggagcattgtttacagctgtgagtggccactgtacatgtggcccttccagaagcccaactacactgagatccgccagtactgcaaccattggaggaactttgcagacatcgatgattcctgggcctccatcaagagcatcctggactggacttccagaaaccaagagagaattgtggatgttgctggacctggaggttggaatgaccctgacatgctggtgattggaaattttgggctgtcctgggaccagcaagtgactcagatggccagctgggccatcatggcagcccctctctttatgtccaatgatctcagacacatttccccccaagccaaggctctgctccaagacaaagatgtcattgctattaatcaagatcccctggggaagcaaggctaccagctgaggaaaggagacaactttgaggtgtgggagagacctctgtctggagatgcctgggctgtggctatcatcaatagacaagaaattggtggccccagatcctacaccatccctgtggcttccctgggcaagggtgtggcctgcaatccagcttgcttcatcacccagctcctcccagtgaagaggcagcttggcttctacaactggacatctaggctcaagtcccacatcaaccccactggcacagtgctgctgcagctggaaaacaccatgcagatgagcctgaaagacctcctgtga

SEQ ID NO: 42 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatggactggctaggaccccaaccatgggttggctgcactgggagaggtttatgtgtaatctggactgtcaagaagaacctgactcctgcatctctgagaagctgttcatggagatggctgagaggatggtgagtgaaggctggaaggatgctggttatgagtacctgtgcattgatgactgctggatggcccctcagagagattcagagggcagacttcaagcagacccccagaggttcccccatggcatccgccagctggccaactatgtccactccaagggcctgaaactgggtatctatgctgatgtgggcaacaagacctgtgctggctttcctggctcctttggctactatgacattgacgcccagacctttgctgactggggtgtggacctcctcaagtttgatggctgctactgtgactctctggaaaacctggcagatggttacaagcacatgtctcttgccctgaacagaactggtaggagcattgtttacagctgtgagtggccactgtacatgtggcccttccagaagcccaactacactgagatcagacagtactgcaaccattggaggaattttgccgacatcgatgattcctgggccagcatcaagtccatcctggactggacttccagaaaccaagagagaattgtggatgttgctggacctggaggatggaatgacccagacatgctggtgattggaaatttcgggctgtcctgggaccagcaagtgactcagatggcctcttgggccatcatggcagcccccctcttcatgtccaatgatcttagacacatttccccacaagccaaggccctcctgcaagacaaagatgtcattgctattaatcaagatcccctggggaagcaaggctaccagctcagaaaaggagacaactttgaggtgtgggaaagacctctgtctggagatgcctgggctgtggctatcatcaatagacaagaaattggtggccccagatcctacaccatccctgtggcctccctgggcaagggtgttgcctgcaatccagcttgcttcatcacccagctgctcccagtgaagaggcagttgggcttctacaactggacatctaggttgaagagccacatcaaccccactggcacagtgctgctgcagctggagaacaccatgcagatgagcctgaaggacctgctttaa

SEQ ID NO: 43 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggactggcaagaaccccaaccatgggttggctgcactgggagaggtttatgtgtaatctggactgtcaagaggagccagactcctgcatctctgagaagctcttcatggagatggctgagaggatggtgagtgaaggctggaaggatgctggttatgagtacctgtgcattgatgactgctggatggcccctcagagagattcagagggcagactgcaagcagatccccagaggttcccccatggcattaggcaactggccaactatgtccactccaagggcctgaagctgggcatctatgctgatgtgggcaacaagacctgtgctggcttccctggctcctttggctactatgatattgatgcccagacctttgctgactggggtgtggacctgctcaagtttgatggctgctactgtgacagcctggagaacctggctgatggttacaagcacatgtctcttgctctgaacagaactggtagaagcattgtttacagctgtgagtggccactgtacatgtggcccttccagaagcccaactacactgagatcagacagtactgcaaccattggaggaactttgcagacattgatgattcctgggcctccatcaagagcatcctggactggacttccagaaaccaagagagaattgtggatgttgctggacctggaggatggaatgaccctgacatgctggtgattggaaattttgggctgtcctgggaccagcaagtgactcagatggccagctgggccatcatggcagcccccctgttcatgagcaatgatctcagacacatttccccccaagccaaggccctgcttcaagacaaagatgtcattgctattaatcaagatcctttgggcaagcaaggctaccagctgaggaagggagacaactttgaggtgtgggaaagacctctgtctggagatgcctgggctgtggctatcatcaatagacaagaaattggtggccccagatcctacaccatccctgttgcctctctgggaaaaggagtggcctgcaatccagcttgcttcatcacccagctcctcccagtgaagaggcagctgggtttctacaactggacaagcagattgaagtcccacatcaaccccactggcacagtgctgctgcagctggaaaacaccatgcagatgtccctgaaggacctcctgtga

SEQ ID NO: 44 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcacttgataatggactggcaagaaccccaaccatgggctggctgcactgggaaagattcatgtgtaatctggactgtcaagaagaaccagattcctgcatctctgagaagctgtttatggagatggctgagaggatggtgtcagaaggatggaaggatgctggttatgagtacctgtgcattgatgactgctggatggccccccagagagacagtgaaggcagacttcaagcagaccctcagaggttcccccatggcattaggcaactagccaactatgtccactccaagggcctgaaactgggcatctatgctgatgtgggcaacaagacctgtgctggcttccctggctcctttggctactatgacattgatgcccagacctttgctgactggggtgtggacctcctcaagtttgatggctgctactgtgactccctggaaaacctggctgatggttacaagcacatgagcctggccctgaacagaactggtaggagcattgtttacagctgtgagtggccactgtacatgtggcccttccagaagcccaactacactgagatcagacagtactgcaaccattggaggaactttgcagatattgatgattcttgggccagcatcaagtccatcctggactggacatccagaaaccaagagagaattgtggatgttgctggacctggaggttggaatgatcctgacatgctggtgattggaaattttgggctgtcctgggaccagcaagtgactcagatggcctcctgggccatcatggcagcccctctcttcatgtccaatgacttgagacacatttccccccaagccaaggccctcctgcaagacaaagatgtcattgctattaatcaagatcccttgggcaagcaaggctaccagctgaggaagggagacaactttgaggtgtgggagaggcccctgtctggagatgcctgggctgtggctatcatcaatagacaagaaattggtggccccagatcctacaccatccctgttgcctctcttggcaaaggagtggcctgcaatccagcttgcttcatcacccagctccttcctgtgaagaggcagctgggtttctacaactggacttcaaggttgaagagccacatcaaccccactggcacagtgctgctgcagctggagaacaccatgcagatgtctctgaaggacctgctttaa

SEQ ID NO: 45 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggactggcaagaaccccaaccatgggctggctgcactgggagaggttcatgtgtaatctggactgtcaagaggagcctgactcctgcatctctgagaagctgtttatggagatggctgagaggatggtgtctgaaggatggaaggatgctggctatgagtacctgtgcattgatgactgctggatggcccctcagagggacagtgaaggcagactgcaagcagacccccagagattcccccatggcattagacagcttgccaactatgtccactccaagggcctgaaactgggcatctatgctgatgtgggcaacaagacctgtgctggtttccctggctcctttggctactatgacattgatgcccagacctttgctgactggggtgtggacctcctcaagtttgatggctgctactgtgacagcctggaaaacctggctgatggttacaagcacatgtctctggccctgaacagaactggtaggagcattgtttacagctgtgagtggccactgtacatgtggcccttccagaagcccaactacactgagatcagacagtactgcaaccattggaggaactttgcagatattgatgattcctgggcctccatcaagagcatcctggactggacttccagaaaccaagagagaattgtggatgtagctggacctggaggttggaatgacccagacatgctggtgattggaaattttgggctgtcctgggaccagcaagtgactcagatggccagctgggccatcatggcagcccccctcttcatgagcaatgatctcagacacatttccccccaagccaaggctctgctccaagacaaagatgtcattgctattaatcaagatcccctggggaagcaaggctaccagctgaggaagggagacaactttgaggtgtgggaaagacctctgtctggagatgcctgggctgtggctatcatcaatagacaagaaattggtggccccagatcctacaccatccctgttgcttctcttggcaaaggagtggcctgcaatccagcttgcttcatcacccagctcctgcctgtgaagaggcagttgggcttctacaactggacatctaggttgaagtcccacatcaaccccactggcacagtgctgctgcagctggagaacaccatgcagatgtccctgaaggacctgctttaa

SEQ ID NO: 46 - protein, 398 aa, Artificial Sequence
  Synthetic polypeptide
LDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRDIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDTDVIAINQDPLGKQGYQLRKGDNFEVWERPLSGDAWAVAIINRQEIGGPRGYTIPVAKLGKGVACNPACFITQLLPVKRKLGFYEATSRLRSHINPTGTVLLQLENTMQTSLKDLL

SEQ ID NO: 47 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFMEMAERMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYEWTSRLKSHINPTGTVLLQLENTMQMSLKDLL

SEQ ID NO: 48 - protein, 429 aa, Artificial Sequence
  Synthetic polypeptide
MQLRNPELHLGCALALRFLALVSWDIPGARALDNGLARTPPMGWLHWERFMCNLDCQEEPDSCISEKLFEEMAERMVTEGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRSYTIPVASLGKGVACNPACFITQLLPVKRKLGFYEATSRLRSHINPTGTVLLQLENTMQTSLKDLL

SEQ ID NO: 49 - protein, 429 aa, Artificial Sequence
  Synthetic polypeptide
MQLRNPELHLGCALALRFLALVSWDIPGARALDNGLARTPPMGWLHWERFMCNLDCQEEPDSCISEKLFEEMAERMVTEGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRSYTIPVASLGKGVACNPACFITQLLPVKRKLGFYEATSRLRSHINPTGTVLLQLENTMQTSLKDLL

SEQ ID NO: 50 - protein, 429 aa, Artificial Sequence
  Synthetic polypeptide
MQLRNPELHLGCALALRFLALVSWDIPGARALDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRGYTIPVASLGKGVACNPACFITQLLPVKRKLGFYEATSRLRSHINPTGTVLLQLENTMQTSLKDLL

SEQ ID NO: 51 - protein, 429 aa, Artificial Sequence
  Synthetic polypeptide
MQLRNPELHLGCALALRFLALVSWDIPGARALDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDKDVIAINQDPLGKQGYQLRKGDNFEVWERPLSGDAWAVAIINRQEIGGPRGYTIPVAKLGKGVACNPACFITQLLPVKRKLGFYEATSRLRSHINPTGTVLLQLENTMQTSLKDLL

SEQ ID NO: 52 - protein, 429 aa, Artificial Sequence
  Synthetic polypeptide
MQLRNPELHLGCALALRFLALVSWDIPGARALDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRDIVYSCEWPLYMWPFQKPNYTEIRQYCNHWRNFADIDDSWASIKSILDWTSRNQERIVDVAGPGGWNDPDMLVIGNFGLSWDQQVTQMALWAIMAGPLFMSNDLRAISPQAKALLQDTDVIAINQDPLGKQGYQLRKGDNFEVWERPLSGDAWAVAIINRQEIGGPRGYTIPVAKLGKGVACNPACFITQLLPVKRKLGFYEATSRLRSHINPTGTVLLQLENTMQTSLKDLL

SEQ ID NO: 53 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSFNQERIVDVAGPGGWNDPDMLVIGNFGLSWDQQVTQMALWAIMAAPLFMSNDLRHISPQAKALLQDKDVIAINQDPLGKQGYQLRQGDNFEVWERPLSGLAWAVAVINRQEIGGPRSYTIAVASLGGGVACNPACFITQLLPVKRKLGLYEWTSRLKSHINPTGTVLLQLENTMQMSLKDLL

SEQ ID NO: 54 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRKGDNFEVWERPLSGDAWAVAMINRQEIGGPRSYTIPVASLGKGVACNPACFITQLLPVKRKLGFYEWTSRLRSHINPTGTVLLQLENTMQMSLKDLL

SEQ ID NO: 55 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFMEMAERMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSRNQERIVDVAGPGGWNDPDMLVIGNFGLSWDQQVTQMASWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 56 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSRNQERIVDVAGPGGWNDPDMLVIGNFGLSWNQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRKLGFYEWTSRLRSHINPTGTVLLQLENTMQMSLKDLL

SEQ ID NO: 57 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFEEMAELMVSEGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGDAWAVAMINRQEIGGPRSYTIPVASLGKGVACNPACFITQLLPVKRKLGFYEWTSRLRSHINPTGTVLLQLENTMQMSLKDLL

SEQ ID NO: 58 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRKLGFYEATSRLKSHINPTGTVLLQLENTMQMSLKDLL

SEQ ID NO: 59 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWASIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 60 - protein, 429 aa, Artificial Sequence
  Synthetic polypeptide
MQLRNPELHLGCALALRFLALVSWDIPGARALDNGLARTPPMGWLHWERFMCNLDCQEEPDSCISEKLFEEMAERMVTDGWKDAGYEYLCIDDCWMAPQRDSEGRLQADPQRFPHGIRQLANHVHSKGLKLGIYADVGNKTCAGFPGSFGYYDIDAQTFADWGVDLLKFDGCYCDSLENLADGYKHMSLALNKTGRPIVYSCEWPLYMWPFQKPNYTEIRQYCNHWRNFADIDDSWASIKSILDWTSRNQERIVDVAGPGGWNDPDMLVIGNFGLSWDQQVTQMALWAIMAAPLFMSNDLRHISPQAKALLQDKDVIAINQDPLGKQGYQLRKGDNFEVWERPLSGDAWAVAIINRQEIGGPRSYTIPVASLGKGVACNPACFITQLLPVKRQLGFYNWTSRLKSHINPTGTVLLQLENTMQMSLKDLL

SEQ ID NO: 61 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggattggctagaacacctactatggggtggcttcactgggagaggttcatgtgcaacctcgactgtcaggaagaaccagacagctgcatctccgagaagctgtttatggaaatggccgagcgaatggtgtcagaaggctggaaagatgcaggttacgagtatctgtgtattgacgattgctggatggctccgcaacgggacagtgagggcagacttcaggcagatcctcagcgcttcccacatgggataaggcagctcgccaactacgtccactctaagggactgaaactgggcatctatgctgacgtggggaataagacctgtgcgggatttcccggtagcttcggctactacgacattgatgcccagacctttgccgattggggagttgacctcctcaaattcgatggctgctattgtgactctttggagaacctggcagacgggtacaagcatatgtccctggccctgaatcggacaggtagacccatcgtgtatagttgcgaatggcccctttacatgtggccttttcaaaagccaaactacactgagattcgccagtattgcaatcactggaggaacttcgctgatatcgatgactcatgggcgagcatcaaatccatattggattggacctctcggaatcaggagcgcattgtagacgtcgcaggacccggcggctggaacgaccctgatatgctggtgatcgggaattttggtcttagctgggaccagcaagttacgcagatggctctgtgggcaattatggcagccccactcttcatgtccaacgatctgcgacacatctctcctcaagctaaggctctgctgcaggacaaagatgtgattgccatcaatcaggacccactcggaaagcagggctatcagctgagaaaaggcgacaacttcgaagtctgggaaaggccactttcaggagacgcatgggctgtggccataataaaccggcaagagattggtgggcccaggagctacacaatccccgttgccagtttgggcaagggagtggcgtgtaatcctgcctgctttatcactcagctgctcccagtcaaaagacagctggggttctatgagtggacctcccgcctcaagagccatattaatcccacaggtaccgtactgctgcaacttgaaaacacgatgcagatgagtttgaaggacctcctgtag

SEQ ID NO: 62 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggattggctagaacacctcctatggggtggcttcactgggagaggttcatgtgcaacctggactgtcaggaagaaccagacagctgcatctccgagaagctctttgaggaaatggccgaacgaatggtgactgagggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaatagaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgctcctatacaattcctgtagcatctcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 63 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggattggctagaacacctcctatggggtggcttcactgggagaggttcatgtgcaacctggactgtcaggaagaaccagacagctgcatctccgagaagctctttgaggaaatggccgaacgaatggtgactgagggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgctcctatacaattcctgtagcatctcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 64 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggattggctagaacacctcctatggggtggcttcactgggagaggttcatgtgcaacctggactgtcaggaagaaccagacagctgcatctccgagaagctctttgaggaaatggccgaacgaatggtgactgacggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgcggttatacaattcctgtagcatctcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 65 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggattggctagaacacctcctatggggtggcttcactgggagaggttcatgtgcaacctggactgtcaggaagaaccagacagctgcatctccgagaagctctttgaggaaatggccgaacgaatggtgactgacggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcggccaatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggtcctcttttcatgagcaacgatctgcgcgcgatctcaccacaggcaaaggccctgctccaagacaaagatgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgcggttatacaattcctgtagcaaagcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 66 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcactggataatggattggctagaacacctcctatggggtggcttcactgggagaggttcatgtgcaacctggactgtcaggaagaaccagacagctgcatctccgagaagctctttgaggaaatggccgaacgaatggtgactgacggctggaaagatgcaggttacgagtatctgtgtattgacgattgctggatggccccacaacgggattctgagggaagacttcaggctgatccgcagcgcttccctcatggcataaggcagctggcaaaccacgtccacagtaaggggctcaaattgggaatctacgcggacgtgggcaataagacctgtgccggttttccgggatcattcgggtattatgacattgacgcccaaacgtttgctgattggggcgttgacctgctgaaattcgatggttgctactgtgacagcctcgaaaacctggcagacggctacaagcatatgtctctcgccctgaataaaaccggtcgggatatcgtatattcctgcgagtggcctctttacatgtggccatttcagaaaccgaactacacagaaattcgccagtattgcaatcattggaggaacttcgctgatatcgatgactcatgggcctccataaagagcatcttggactggaccagtcggaatcaggagcgaattgtggatgtcgcaggccctggaggatggaacgatccagacatgctggtgatcggcaattttggcctctcttgggaccagcaggttacccaaatggctctgtgggcaattatggccggccctcttttcatgagcaacgatctgcgcgcgatctcaccacaggcaaaggccctgctccaagacacggacgtgatagccatcaatcaggacccgttgggaaagcagggctaccagctgagaaaaggcgacaactttgaggtctgggaaaggcctttgagtggagatgcgtgggctgtggccattattaatcggcaagagatcggaggtccacgcggttatacaattcctgtagcaaagcttggcaagggcgttgcctgtaacccagcttgcttcatcactcagcttctgccggtgaagagaaaactgggtttctacgaagctaccagcaggctccgatcccacatcaaccctacaggaaccgtcctcttgcagctggagaatacgatgcagacttcactgaaggacctcctgtag

SEQ ID NO: 67 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatgggctggccaggacacctactatgggctggctccactgggagcgctttatgtgtaacctcgactgccaagaggagccagactcatgcatctctgagaagttgttcatggagatggctgagctgatggtgagcgaagggtggaaggatgcgggctatgagtatctctgtattgatgactgctggatggctccacagcgcgacagtgaaggccggctccaggccgatcctcagcggttcccccacggtatcagacaactggcgaattacgtgcactcaaaaggccttaagctgggtatatatgctgatgtgggtaataaaacatgtgcaggcttcccaggctcttttgggtactatgacatcgacgcccagacttttgcggactggggcgtggacctgctcaagtttgacggatgttactgtgactcccttgagaacctggccgacgggtacaagcatatgtcactggccctgaatcggacaggccgatccatcgtatactcttgcgagtggcctctgtacatgtggcccttccagaagcccaactatacagaaatcaggcaatactgcaaccattggcggaacttcgcagacatagacgacagctgggctagcattaagtctattctggattggaccagtttcaatcaagaaaggattgtcgatgtcgcaggcccaggaggttggaatgacccagacatgctcgtgattggaaatttcggtctgtcatgggaccaacaggtgactcagatggctctgtgggcaatcatggcagcaccactgttcatgagcaatgatttgcgacacatctcccctcaggcgaaagcccttctgcaggataaggacgttatcgccattaaccaggacccgctcggtaagcaagggtaccagttgcgccagggagacaatttcgaggtctgggaacgacccctgtctggactcgcttgggccgtagccgtaattaaccgacaagaaatcggcggaccgcggagctataccatagctgtcgcctccctcggcgggggcgtagcttgtaacccggcttgtttcataacccagctgctgcccgtcaagaggaaactggggctttatgagtggacaagtcggttgaagtctcatattaacccgacagggactgttctcctccagctggaaaacacaatgcagatgagcctgaaggatctcttgtga

SEQ ID NO: 68 - DNA, 1291 bp, Artificial Sequence
  Synthetic polynucleotide
atgcagctgaggaacccagaactacatctgggctgcgcgcttgcgcttcgcttcctggccctcgtttcctgggacatccctggggctagagcatttggacaatgggctggccaggacacctactatgggctggctccactgggagcgctttatgtgtaacctcgactgccaagaggagccagactcatgcatctctgagaagttgttcatggaaatggccgagcttatggtctcagaggggtggaaagacgccggatatgagtatctgtgcatcgacgattgctggatggccccccaacgcgattccgaaggccgcttgcaggctgatccgcagcgctttcctcacgggatccgccaattggcaaattacgtgcactcaaaggggctcaagttgggcatctacgcagacgtgggaaacaagacatgtgctggatttcccgggagtttcggttattatgacattgacgcacagacctttgctgattggggggtcgacctcctgaagtttgacggttgttattgcgacagccttgaaaatctggccgacggttacaaacacatgtcccttgcactgaatagaactggtcggtccatcgtctatagttgtgagtggccgctttacatgtggccttttcagaaacccaactacaccgagattcggcaatattgcaatcactggcgaaatttcgcagatatcgatgattcttgggctagtattaaatccatcctggattggacatcattcaaccaggagcgcatcgtggacgttgctggacctggcgggtggaatgatccagacatgcttgtgatcggaaacttcggtctctcttggaaccagcaagtcactcaaatggcactctgggcaattatggccgcccccctctttatgtccaacgatctgaggcatatcagtcctcaggctaaagccctgctgcaagacaaggatgtgattgctatcaaccaagatccccttggtaaacaggggtaccagttgcgcaaaggcgacaattttgaggtgtgggaaaggccactttcaggcgatgcatgggccgttgcaatgatcaacaggcaagaaattggcggacccaggagctatacaataccagtggcgtcactgggtaagggagtcgcctgtaaccccgcatgcttcattactcaacttctgccagtgaaacgaaaactcggattctatgagtggacctcaagactgaggtctcatattaacccgacagggactgttctcctccagctggaaaacacaatgcagatgagcctgaaggatctcttgtga

SEQ ID NO: 69 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatgggctggccaggacacctactatgggctggctccactgggagcgctttatgtgtaacctcgactgccaagaggagccagactcatgcatctctgagaagttgttcatggagatggctgaacgcatggtttctgagggatggaaagatgcaggctacgagtacctgtgtatagacgattgttggatggccccacagcgggattcagaaggtagactccaggcggatccccagagattcccacatggaatcagacagctggccaattacgtccattccaaaggccttaagttgggtatttacgccgacgtaggcaacaagacttgtgccggatttcccggcagtttcggatactatgacattgatgcacagactttcgctgactggggggtggacttgctcaaatttgatggctgttattgcgatagcctcgaaaatctggctgatggctacaagcacatgtcactcgctctcaaccgcactgggcgctctatagtttactcctgcgagtggcctctgtatatgtggccgttccagaaacccaattacacagaaataaggcagtattgcaatcactggcgcaactttgctgatattgatgattcctgggcctccataaagagtatcttggactggactagtcgcaatcaggaaagaattgtcgacgtcgccggaccaggcggatggaatgatcctgatatgctcgtgatcgggaacttcggactctcatgggaccagcaggtgacccagatggctagttgggctatcatggccgcccctctgtttatgagtaacgacctccgccacatcagcccccaggccaaggcgcttctgcaggataaagatgtcatcgccatcaaccaagatcccctgggcaaacaaggctatcagctgcggaagggagacaattttgaggtgtgggaacgccctttgagcggagacgcctgggctgtggctattataaatcgccaggagattgggggcccgagaagttatactatccccgttgcaagcctgggaaagggcgtcgcctgcaaccctgcctgcttcatcacacagctgcttcctgtcaaacgccaattggggttctacaattggacatccagactcaaatctcatattaacccgacagggactgttctcctccagctggaaaacacaatgcagatgagcctgaaggatctcttgtga

SEQ ID NO: 70 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatgggctggccaggacacctactatgggctggctccactgggagcgctttatgtgtaacctcgactgccaagaggagccagactcatgcatctctgagaagttgttcgaggagatggcagaacgaatggtgacagatggatggaaggacgctggctacgagtatctgtgcatagatgattgttggatggcccctcagcgagactcagaggggagactccaggccgacccccagcgatttccacacggaatccggcaactggctaaccatgtgcactcaaaagggctcaagctgggaatttatgctgacgtcgggaacaaaacttgtgcggggtttcccggctccttcggatattacgacatcgacgcccagactttcgcagactggggtgtggacctgcttaagttcgacggctgttactgcgatagtctggaaaacttggctgacggctataagcacatgagtctcgccctgaaccgaacaggcagaagcatagtctactcctgcgaatggccactttacatgtggccattccagaaacctaattataccgagatcagacaatactgtaaccattggcgaaacttcgccgacattgacgatagttggaagtcaatcaagtccatcctggattggacctctaggaaccaggaaaggatcgtggacgtggctggacctggcggatggaacgatccagacatgctcgtgataggaaactttggactgtcatggaatcagcaagtaacacagatggcgctctgggccattatggctgcccccttgtttatgtctaacgacctgaggcatatctctcctcaagccaaggcactcctgcaggacaaggacgttatcgccatcaaccaggacccactgggcaagcagggataccagctgcggaaaggtgataacttcgaggtctgggagcgaccgctttcaggagacgcctgggcagttgcaatcatcaacaggcaagaaattggtgggccacggtcttatactattcccgtggcttctctcggtaagggcgtcgcctgcaaccccgcctgctttatcacccaattgttgcccgttaagagaaaactgggattttacgagtggacatccaggctgcggtctcatattaacccgacagggactgttctcctccagctggaaaacacaatgcagatgagcctgaaggatctcttgtga

SEQ ID NO: 71 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatgggctggccaggacacctactatgggctggctccactgggagcgctttatgtgtaacctcgactgccaagaggagccagactcatgcatctctgagaagttgttcgaagagatggctgaactgatggtgtccgaggggtggaaggatgcagggtacgagtacctttgcatagacgattgctggatggcaccacagcgggatagtgagggcaggttgcaggcggacccccaaagatttccacatggcatcagacagctggccaaccacgtgcactctaaaggcctgaagctggggatttacgccgatgtcggtaataaaacatgtgctggtttccccggtagctttggatactacgacatcgacgcccagacatttgctgattggggagtggacctgctcaagttcgacggctgctactgcgattctctggaaaatctggccgatgggtataagcacatgagtcttgctcttaatagaaccggacgctctatagtctattcatgtgagtggccactgtacatgtggccatttcagaaacccaactataccgaaattagacagtattgtaaccactggcggaatttcgccgacatagacgatagctggaagagcatcaagtcaattctcgattggacttcattcaaccaggagcggatcgtcgacgtggccggccctgggggctggaatgatccagatatgctggtgatcggaaactttggtctctcctggaatcaacaggtcactcagatggccctctgggccattatggcagcaccactctttatgagcaacgacctgagacacatttccccacaggccaaggcattgctgcaggacaaagacgtgatcgccattaaccaggacccactgggcaagcaagggtaccaacttaggcagggagataattttgaggtctgggagcgcccactcagcggagacgcctgggccgttgccatgataaacagacaggagattggcggccccagatcctacacaattcctgtcgctagcctgggcaagggggtggcttgtaatcccgcctgctttataacccagctgctgccagtgaaacggaaactcgggttctatgaatggacttctaggctcaggtctcatattaacccgacagggactgttctcctccagctggaaaacacaatgcagatgagcctgaaggatctcttgtga

SEQ ID NO: 72 - DNA, 1290 bp, Artificial Sequence
  Synthetic polynucleotide
atgcagctgaggaacccagaactacatctgggctgcgcgcttgcgcttcgcttcctggccctcgtttcctgggacatccctggggctagagcattggacaatgggctggccaggacacctactatgggctggctccactgggagcgctttatgtgtaacctcgactgccaagaggagccagactcatgcatctctgagaagttgttcgaggagatggcagaacgaatggtgacagatggatggaaggacgctggctacgagtatctgtgcatagatgattgttggatggcccctcagcgagactcagaggggagactccaggccgacccccagcgatttccacacggaatccggcaactggctaaccatgtgcactcaaaagggctcaagctgggaatttatgctgacgtcgggaacaaaacttgtgcggggtttcccggctccttcggatattacgacatcgacgcccagactttcgcagactggggtgtggacctgcttaagttcgacggctgttactgcgatagtctggaaaacttggctgacggctataagcacatgagtctcgccctgaaccgaacaggcagaagcatagtctactcctgcgaatggccactttacatgtggccattccagaaacctaattataccgagatcagacaatactgtaaccattggcgaaacttcgccgacattgacgatagttggaagtcaatcaagtccatcctggattggacctctaggaaccaggaaaggatcgtggacgtggctggacctggcggatggaacgatccagacatgctcgtgataggaaactttggactgtcatgggatcagcaagtaacacagatggcgctctgggccattatggctgcccccttgtttatgtctaacgacctgaggcatatctctcctcaagccaaggcactcctgcaggacaaggacgttatcgccatcaaccaggacccactgggcaagcagggataccagctgcggaaaggtgataacttcgaggtctgggagcgaccgctttcaggagacgcctgggcagttgcaatcatcaacaggcaagaaattggtgggccacggtcttatactattcccgtggcttctctcggtaagggcgtcgcctgcaaccccgcctgctttatcacccaattgttgcccgttaagagaaaactgggattttacgaggcgacatccaggctgaagtctcatattaacccgacagggactgttctcctccagctggaaaacacaatgcagatgagcctgaaggatctcttgtga

SEQ ID NO: 73 - DNA, 1289 bp, Artificial Sequence
  Synthetic polynucleotide
atgcagctgaggaacccagaactacatctgggctgcgcgcttgcgcttcgcttcctggccctcgtttcctgggacatccctggggctagagcatggacaatggattggcaaggacgcctaccatgggctggctgcactgggagcgcttcatgtgcaaccttgactgccaggaagagccagattcctgcatcagtgagaagctcttcgaggagatggcagagagaatggtcaccgacggctggaaggatgcaggttatgagtacctctgcattgatgactgttggatggctccccaaagagattcagaaggcagacttcaggcagaccctcagcgctttcctcatgggattcgccagctagctaatcacgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctgggccagtataaagagtatcttggactggacatctagaaaccaggagagaattgttgatgttgctggaccagggggttggaatgacccagatatgttagtgattggcaactttggcctcagctgggaccagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagaaagggagacaactttgaagtgtgggaacgacctctctcaggcgatgcctgggctgtagctatcataaaccggcaggagattggtggacctcgctcttataccatccccgttgcttccctgggtaaaggagtggcctgtaatcctgcctgcttcatcacacagctcctccctgtgaaaaggcagctagggttctataactggacttcaaggttaaagagtcacataaatcccacaggcactgttttgcttcagctagaaaatacaatgcagatgtcattaaaagacttactttaa

SEQ ID NO: 74 - DNA, 1289 bp, Artificial Sequence
  Synthetic polynucleotide
atgcagctgaggaacccagaactacatctgggctgcgcgcttgcgcttcgcttcctggccctcgtttcctgggacatccctggggctagagcatggacaatggattggcaaggacgcctcccatgggctggctgcactgggagcgcttcatgtgcaaccttgactgccaggaagagccagattcctgcatcagtgagaagctcttcgaggagatggcagagagaatggtcaccgacggctggaaggatgcaggttatgagtacctctgcattgatgactgttggatggctccccaaagagattcagaaggcagacttcaggcagaccctcagcgctttcctcatgggattcgccagctagctaatcacgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataagactggcagacccattgtgtactcctgtgagtggcctctttatatgtggccctttcaaaagcccaattatacagaaatccgacagtactgcaatcactggcgaaattttgctgacattgatgattcctgggccagtataaagagtatcttggactggacatctagaaaccaggagagaattgttgatgttgctggaccagggggttggaatgacccagatatgttagtgattggcaactttggcctcagctgggaccagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagaaagggagacaactttgaagtgtgggaacgacctctctcaggcgatgcctgggctgtagctatcataaaccggcaggagattggtggacctcgctcttataccatccccgttgcttccctgggtaaaggagtggcctgtaatcctgcctgcttcatcacacagctcctccctgtgaaaaggcagctagggttctataactggacttcaaggttaaagagtcacataaatcccacaggcactgttttgcttcagctagaaaatacaatgcagatgtcattaaaagacttactttaa

SEQ ID NO: 75 - protein, 429 aa, Artificial Sequence
  Synthetic polypeptide
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 76 - protein, 31 aa, Artificial Sequence
  Synthetic polypeptide
MQLRNPELHLGCALALRFLALVSWDIPGARA

SEQ ID NO: 77 - DNA, 93 bp, Artificial Sequence
  Synthetic polynucleotide
atgcagctgaggaacccagaactacatctgggctgcgcgcttgcgcttcgcttcctggccctcgtttcctgggacatccctggggctagagca